(19) United States
(12) Patent Application Publication
(10) Pub. No.: US 2026/0274805 A1
(43) Pub. Date: Sep. 17, 2026

(54) COMPOSITIONS COMPRISING 2,2,4-TRIMETHYL-N-PHENYL-1,2- DIHYDROQUINOLIN-6-AMINE

(71) Applicant: FLEXSYS IP HOLDINGS, LLC, Akron, OH (US)

(72) Inventors: Malik Hani AL-AFYOUNI, Akron, OH (US); Judicael Jacques CHAPELET, Copley, OH (US); Donald L. FIELDS, Jr., Copley, OH (US); Frederick IGNATZ-HOOVER, Elyria, OH (US); Jonathan Michael PENNEY, Cuyahoga Falls, OH (US); Andrew Neil SMITH, Wadsworth, OH (US); Ralph D. TRIPLETT, II, Wadsworth, OH (US); Joshua TYHURST, Medina, OH (US); Taylor Blue HAMBY, Cuyahoga Falls, OH (US)

(73) Assignee: FLEXSYS IP HOLDINGS, LLC, Akron, OH (US)

(21) Appl. No.: 19/164,046

(22) PCT Filed: Mar. 8, 2024
(86) PCT No.: PCT/US2024/019142

§ 371 Date: Sep. 10, 2025

Related U.S. Application Data
(60) Provisional application No. 63/489,722, filed on Mar. 10, 2023.
(60) Provisional application No. 63/601,094, filed on Nov. 20, 2023.
(60) Provisional application No. 63/612,249, filed on Dec. 19, 2023.

(51) Int. Cl. C07D 243/14; C07C 211/55; C07D 235/08; C07D 241/46; C08K 5/18; C08K 5/3437

(52) U.S. Cl. CPC C07D 243/14; C07C 211/55; C07D 235/08; C07D 241/46; C08K 5/18; C08K 5/3437

(57) ABSTRACT

The present disclosure provides compounds and compositions with antidegradant, e.g., antiozonant, antioxidant and/or antifatigue, properties that are useful additives for vulcanized rubber articles. In some embodiments, the compositions comprise 2,2,4-trimethyl-N-phenyl-1,2-dihydroquinolin-6-amine.

### BACKGROUND

### Field

The present disclosure provides compounds and compositions with antidegradant, e.g., antiozonant, antioxidant and/or antifatigue, properties that are useful additives for vulcanized rubber articles. In some embodiments, the compositions comprise 2,2,4-trimethyl-N-phenyl-1,2-dihydroquinolin-6-amine.

### Background

Many materials such as plastics, elastomers, elastomeric products (tires, belts, hoses, bushings, mounts, vibration isolators, etc.), lubricants and petroleum products (such as hydraulic fluids, oils, fuels and oil/fuel additives for automotive and aviation applications) are prone to degradation upon prolonged exposure to light, heat, oxygen, ozone, repetitive mechanical actions and the like. Accordingly, compounds and compositions demonstrating antidegradant efficacy are well known in the art. For example, U.S. Pat. No. 8,987,515 discloses an aromatic polyamine useful in inhibiting oxidative degradation particularly in lubricant compositions. U.S. Patent Application Publication No. 2014/0316163 discloses antioxidant macromolecules with purported improved solubility in many commercially available oils and lubricants.

Antidegradants useful in the manufacture of articles formed from elastomers, plastics and the like require a very specific combination of qualities that can be difficult to achieve. While the antidegradants must obviously have commercially acceptable efficacy, they must also exhibit that efficacy over prolonged periods of time associated with use of the article, particularly at exposed surfaces of the article where degradation from environmental factors such as light, oxygen and ozone primarily occurs. Just as important to the protection of surface exposed components, efficacy in protecting imbedded components of composite materials from the effects of oxidative aging and repetitive mechanical action are critically important. The antidegradants must achieve these results while not negatively impacting other additives' efficacy or desirable characteristics in the final article. Further, antidegradants which provide or improve the mechanical fatigue life after an article has been in service, aged oxidatively or by exposure to ozone are highly valued since these will inherently improve the useful mechanical service life of article. Consequently, elastomeric articles which undergo repeated mechanical flexure, extension, or compression during service would greatly benefit from such a discovery.

Articles formed from general purpose elastomers such as natural rubber, in particular tires, are especially prone to degradation from both oxygen and ozone. As discussed in U.S. Pat. No. 2,905,654, the effect on rubber from degradation by oxygen is different from the effect from degradation from ozone; however, both effects can be detrimental to tire performance, appearance and life expectancy. Fatigue and crack propagation are also issues of specific concern, in particular for steel belt edge areas and tire sidewalls which are subject to significant stresses and stretching forces while flexed whether inflated, partially inflated, uninflated and throughout the service life of the tire. U.S. Pat. No. 8,833,417 describes an antioxidant system that purportedly increases long-term resistance to fatigue and crack propagation over the known antioxidants discussed immediately below.

Materials with antidegradant efficacy are well known in the art for use in tire applications and are commercially available. For example, N,N′-disubstituted-paraphenylenediamines such as those sold under the trademark Santoflex™ are generally favored by many tire manufacturers for this purpose. EP 3147321 A1 discloses rubber compositions, tires, amine compounds, and anti-aging agents, and in particular, a rubber composition that is said to be suitable for use in tread rubber or sidewall rubber of a tire. U.S. Pat. No. 3,362,930 discloses 6-anilino-2,2,4-trimethyl-1,2-dihydroquinoline and its use as an antiozonant in a rubber composition. U.S. Pat. No. 5,117,063 discloses a method of making 4-aminodiphenylamine (“4-ADPA”), and 4-ADPA can be used to make 6-anilino-2,2,4-trimethyl-1,2-dihydroquinoline. There exists a need for compositions comprising 6-anilino-2,2,4-trimethyl-1,2-dihydroquinoline (also referred to as 2,2,4-trimethyl-N-phenyl-1,2-dihydroquinolin-6-amine) and other antidegradants for use in vulcanized rubber articles.

### BRIEF SUMMARY

The present disclosure provides compounds and compositions that are useful as antidegradants, e.g., antiozonants, and/or antioxidants, and/or as additives in vulcanized rubber articles, lubricants, or combustible fuels.

In one aspect, the present disclosure provides a compound having Formula (I):

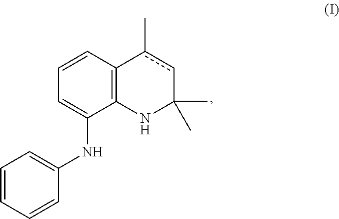

or a salt or solvate thereof, wherein [symbol] represents a single or double bond.

In another aspect, the present disclosure provides a compound having Formula (II):

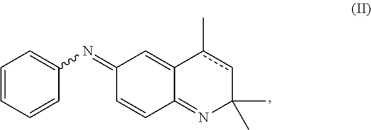

or a salt or solvate thereof, wherein [symbol] represents a single or double bond.

In another aspect, the present disclosure provides a compound having Formula (III):

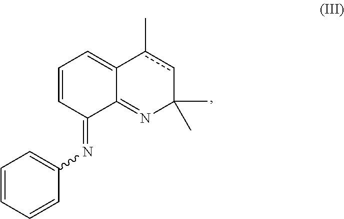

or a salt or solvate thereof, wherein [symbol] represents a single or double bond.

In another aspect, the present disclosure provides Compound (21):

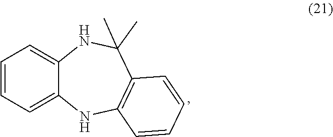 [SMILES: CC1(C)Nc2ccccc2Nc2ccccc21]

or a salt or solvate thereof.

In another aspect, the present disclosure provides Compound (22):

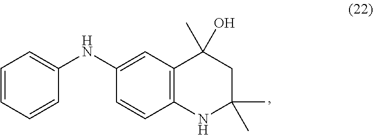 [SMILES: CC1(C)CC(C)(O)c2cc(Nc3ccccc3)ccc2N1]

or a salt or solvate thereof.

In another aspect, the present disclosure provides Compound (23):

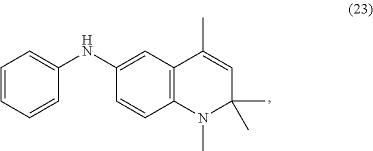 [SMILES: CC1=CC(C)(C)N(C)c2ccc(Nc3ccccc3)cc21]

or a salt or solvate thereof.

In another aspect, the present disclosure provides Compound (24):

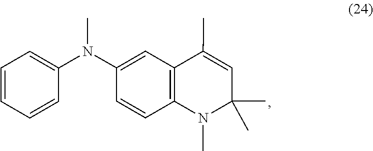 [SMILES: CC1=CC(C)(C)N(C)c2ccc(N(C)c3ccccc3)cc21]

or a salt or solvate thereof.

In another embodiment, the present disclosure provides Compound (25):

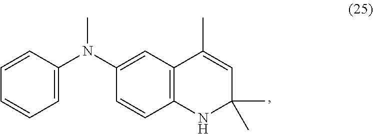 [SMILES: CC1=CC(C)(C)Nc2ccc(N(C)c3ccccc3)cc21]

or a salt or solvate thereof.

In another aspect, the present disclosure provides a composition comprising a compound having Formula (IV):

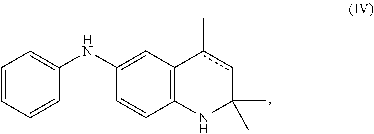

or a salt or solvate thereof, wherein [symbol] represents a single or double bond.

In another aspect, the present disclosure provides a process for preparing a composition comprising Formula (IV), or a salt or solvate thereof, the process comprising reacting 4-aminodiphenylamine with acetone, 2,2-dimethoxypropane, or mesityl oxide in the presence of a strong acid, e.g., hydrochloric acid.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE DRAWINGS

FIG. 1 is a high-performance liquid chromatography (HPLC) chromatograph of a representative Composition of the Disclosure that has been annotated to include the peak locations of certain Compounds of the Disclosure.

FIG. 2 is two chromatograms illustrating the chromatographic detection of Compound (2) in the mixture with Compound (10) (top) and the chromatogram of authentic (2) (bottom) using Method 3.

FIG. 3 is two chromatograms illustrating the chromatographic detection of Compound (21) in the mixture with Compound (10) (top) and the chromatogram of authentic (21) (bottom) using Method 3.

FIG. 4 is two chromatograms illustrating the chromatographic detection of Compound (23) in the mixture with Compound (10) (top) and the chromatogram of authentic (23) (bottom) using Method 3.

FIG. 5 is two chromatograms illustrating the chromatographic detection of Compound (25) in the mixture with Compound (10) (top) and the chromatograms of authentic (25) (bottom) using Method 3.

FIG. 6 is two chromatograms illustrating the chromatographic detection of Compound (21) in the mixture with Compound (10) (top) and the chromatogram of authentic (21) (bottom) using Method 2.

FIG. 7 is two chromatograms illustrating the chromatographic detection of Compound (25) in the mixture with Compound (10) (top) and (Bottom) the chromatogram of authentic (25) using Method 2.

FIG. 8 is an NMR spectrum of Compound (4).

FIG. 9 is a HPLC chromatogram of Compound (4).

FIG. 10 is a 13 APT NMR spectrum of Compound (2).

FIG. 11 is a HPLC chromatogram of Compound (2).

FIG. 12 is a 13 APT NMR spectrum of Compound (21).

FIG. 13 is a HPLC chromatogram of Compound (21).

FIG. 14 is a 13 APT NMR spectrum of Compound (24).

FIG. 15 is a HPLC chromatogram of Compound (24).

FIG. 16 is a 13 APT NMR spectrum of Compound (23).

FIG. 17 is a HPLC chromatogram of Compound (23).

FIG. 18 is a 13 APT NMR spectrum of Compound (7).

FIG. 19 is a HPLC chromatogram of Compound (7).

FIG. 20 is a proton NMR spectrum of Compound (25)

FIG. 21 is a 13 APT NMR spectrum of Compound (25).

FIG. 22 is a HPLC chromatogram of Compound (25).

FIG. 23 is two chromatograms illustrating the chromatographic detection of Compound (37) in the mixture with Compound (10) (top) and the chromatograms of authentic (37) (bottom) using Method 3.

### DETAILED DESCRIPTION

### I. Compounds of the Disclosure

In one embodiment, the present disclosure provides a compound having Formula (I), or a salt or solvate thereof:

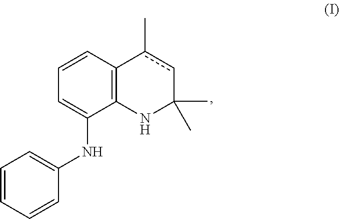

wherein [symbol] represents a single or double bond.

In another embodiment, the present disclosure provides a compound having Formula (I), wherein [symbol] represents a single bond, i.e., Compound (1), or a salt or solvate thereof:

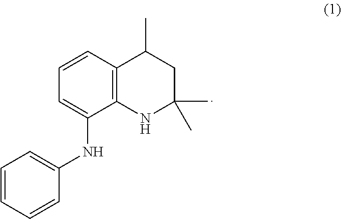 [SMILES: CC1CC(C)(C)Nc2c(Nc3ccccc3)cccc21]

In another embodiment, the present disclosure provides a compound having Formula (I), wherein [symbol] represents a double bond, i.e., Compound (2), or a salt or solvate thereof:

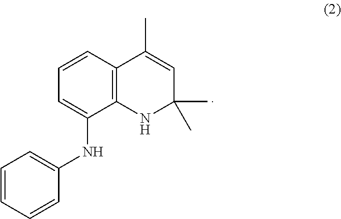 [SMILES: CC1=CC(C)(C)Nc2c(Nc3ccccc3)cccc21]

In another embodiment, the present disclosure provides a compound having Formula (II):

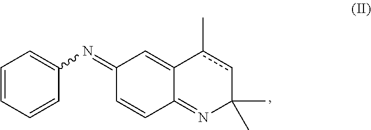

or a salt or solvate thereof, wherein [symbol] represents a single or double bond.

In another embodiment, the present disclosure provides a compound having Formula (II), wherein [symbol] represents a single bond, i.e., Compound (3), or a salt or solvate thereof:

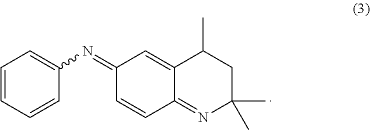 [SMILES: CC1CC(C)(C)N=C2C=CC(=Nc3ccccc3)C=C21]

In another embodiment, the present disclosure provides a compound having Formula (II), wherein [symbol] represents a double bond, i.e., Compound (4), or a salt or solvate thereof:

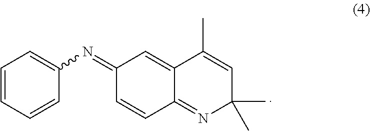 [SMILES: CC1=CC(C)(C)N=C2C=CC(=Nc3ccccc3)C=C12]

In another embodiment, the present disclosure provides a compound having Formula (III):

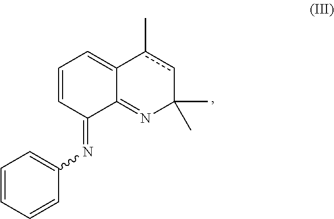

or a salt or solvate thereof, wherein [symbol] represents a single or double bond.

In another embodiment, the present disclosure provides a compound having Formula (III), wherein [symbol] represents a single bond, i.e., Compound (5), or a salt or solvate thereof:

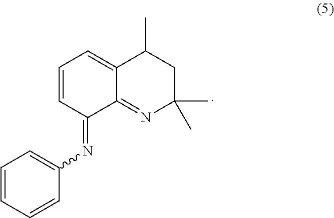 [SMILES: CC1CC(C)(C)N=C2C1=CC=CC2=Nc1ccccc1]

In another embodiment, the present disclosure provides a compound having Formula (III), wherein [symbol] represents a double bond, i.e., Compound (6), or a salt or solvate thereof:

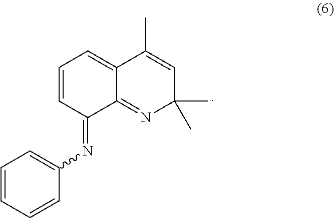 [SMILES: CC1=CC(C)(C)N=C2C1=CC=CC2=Nc1ccccc1]

In another embodiment, the present disclosure provides Compound (21):

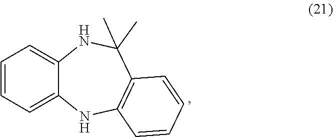 [SMILES: CC1(C)Nc2ccccc2Nc2ccccc21]

or a salt or solvate thereof.

In another embodiment, the present disclosure provides Compound (22):

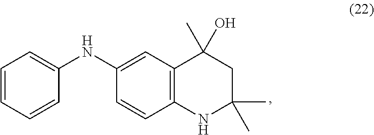 [SMILES: CC1(C)CC(C)(O)c2cc(Nc3ccccc3)ccc2N1]

or a salt or solvate thereof.

In another embodiment, the present disclosure provides Compound (23):

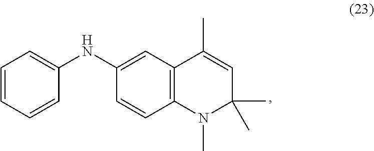 [SMILES: CC1=CC(C)(C)N(C)c2ccc(Nc3ccccc3)cc21]

or a salt or solvate thereof.

In another embodiment, the present disclosure provides Compound (24):

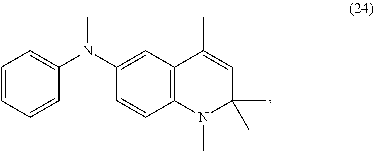 [SMILES: CC1=CC(C)(C)N(C)c2ccc(N(C)c3ccccc3)cc21]

or a salt or solvate thereof.

In another embodiment, the present disclosure provides Compound (25):

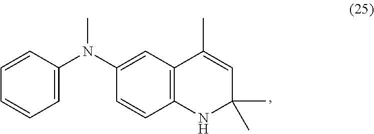 [SMILES: CC1=CC(C)(C)Nc2ccc(N(C)c3ccccc3)cc21]

or a salt or solvate thereof.

In another embodiment, the present disclosure provides a compound of Table 1, or a salt or solvate thereof.

TABLE 1

| Compound | Structure |
|---|---|
| (1) | 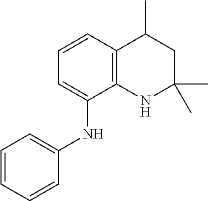 [SMILES: CC1CC(C)(C)Nc2c(Nc3ccccc3)cccc21] |
| (2) | 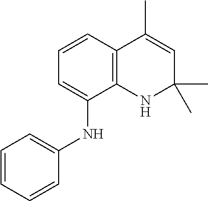 [SMILES: CC1=CC(C)(C)Nc2c(Nc3ccccc3)cccc21] |
| (3) | 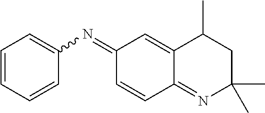 [SMILES: CC1CC(C)(C)N=C2C=CC(=Nc3ccccc3)C=C21] |
| (4) | 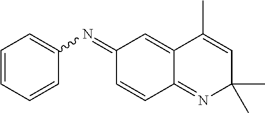 [SMILES: CC1=CC(C)(C)N=C2C=CC(=Nc3ccccc3)C=C12] |
| (5) | 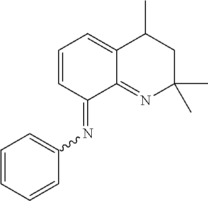 [SMILES: CC1CC(C)(C)N=C2C1=CC=CC2=Nc1ccccc1] |
| (6) | 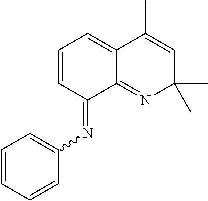 [SMILES: CC1=CC(C)(C)N=C2C1=CC=CC2=Nc1ccccc1] |
| (7) | 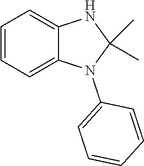 [SMILES: CC1(C)Nc2ccccc2N1c1ccccc1] |
| (8) | 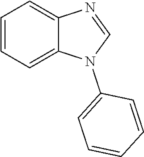 [SMILES: c1ccc(-n2cnc3ccccc32)cc1] |
| (9) | 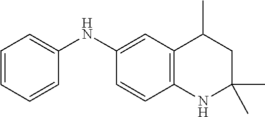 [SMILES: CC1CC(C)(C)Nc2ccc(Nc3ccccc3)cc21] |
| (10) | 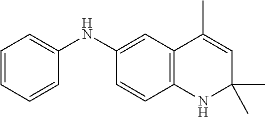 [SMILES: CC1=CC(C)(C)Nc2ccc(Nc3ccccc3)cc21] |
| (11A) | 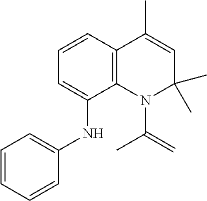 [SMILES: C=C(C)N1c2c(Nc3ccccc3)cccc2C(C)=CC1(C)C] |
| (11B) | 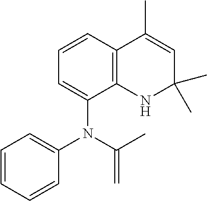 [SMILES: C=C(C)N(c1ccccc1)c1cccc2c1NC(C)(C)C=C2C] |
| (12) | 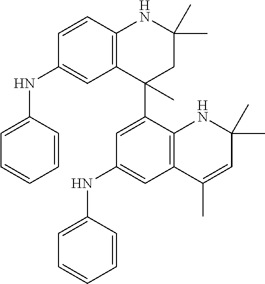 [SMILES: CC1=CC(C)(C)Nc2c1cc(Nc1ccccc1)cc2C1(C)CC(C)(C)Nc2ccc(Nc3ccccc3)cc21] |
| (13) | 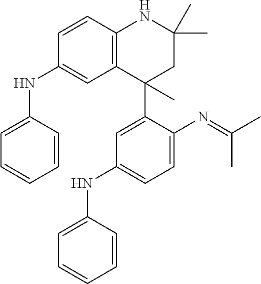 [SMILES: CC(C)=Nc1ccc(Nc2ccccc2)cc1C1(C)CC(C)(C)Nc2ccc(Nc3ccccc3)cc21] |
| (14) | 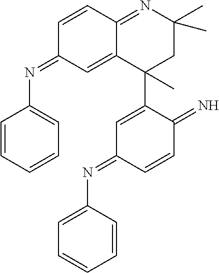 [SMILES: CC1(C)CC(C)(C2=C/C(=N/c3ccccc3)C=CC2=N)C2=C/C(=N\c3ccccc3)C=CC2=N1] |
| (15) | 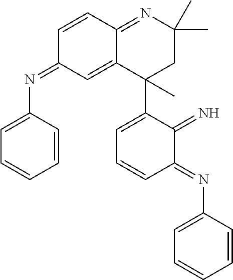 [SMILES: CC1(C)CC(C)(C2=CC=C/C(=N\c3ccccc3)C2=N)C2=C/C(=N\c3ccccc3)C=CC2=N1] |
| (16) | 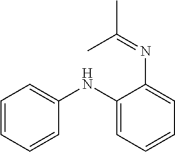 [SMILES: CC(C)=Nc1ccccc1Nc1ccccc1] |
| (17) | 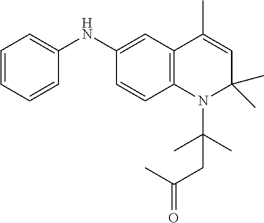 [SMILES: CC(=O)CC(C)(C)N1c2ccc(Nc3ccccc3)cc2C(C)=CC1(C)C] |
| (18) | 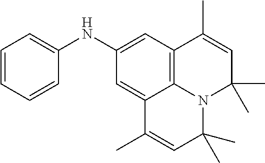 [SMILES: CC1=CC(C)(C)N2c3c1cc(Nc1ccccc1)cc3C(C)=CC2(C)C] |
| (19) | 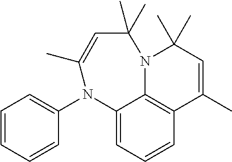 [SMILES: CC1=CC(C)(C)N2c3c1cccc3N(c1ccccc1)C(C)=CC2(C)C] |
| (20A) | 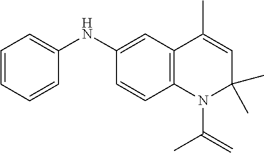 [SMILES: C=C(C)N1c2ccc(Nc3ccccc3)cc2C(C)=CC1(C)C] |
| (20B) | 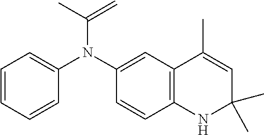 [SMILES: C=C(C)N(c1ccccc1)c1ccc2c(c1)C(C)=CC(C)(C)N2] |
| (21) | 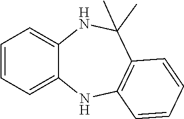 [SMILES: CC1(C)Nc2ccccc2Nc2ccccc21] |
| (22) | 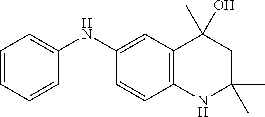 [SMILES: CC1(C)CC(C)(O)c2cc(Nc3ccccc3)ccc2N1] |
| (23) | 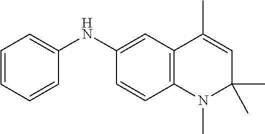 [SMILES: CC1=CC(C)(C)N(C)c2ccc(Nc3ccccc3)cc21] |
| (24) | 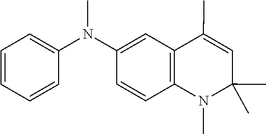 [SMILES: CC1=CC(C)(C)N(C)c2ccc(N(C)c3ccccc3)cc21] |
| (25) | 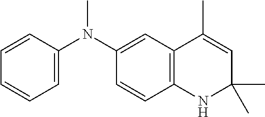 [SMILES: CC1=CC(C)(C)Nc2ccc(N(C)c3ccccc3)cc21] |
| (26) | 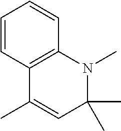 |
| (27a) | 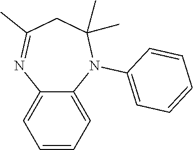 [SMILES: CC1=Nc2ccccc2N(c2ccccc2)C(C)(C)C1] |
| (27b) | 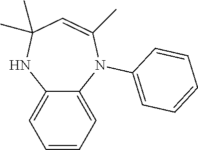 [SMILES: CC1=CC(C)(C)Nc2ccccc2N1c1ccccc1] |
| (28) | 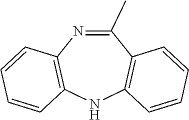 [SMILES: CC1=Nc2ccccc2Nc2ccccc21] |
| (29) | 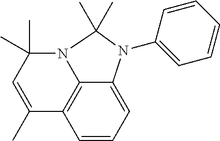 [SMILES: CC1=CC(C)(C)N2c3c1cccc3N(c1ccccc1)C2(C)C] |
| (30) | 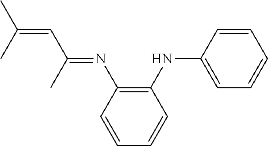 [SMILES: CC(C)=C/C(C)=N/c1ccccc1Nc1ccccc1] |
| (31) | 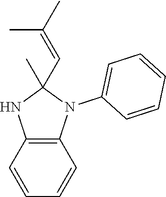 [SMILES: CC(C)=CC1(C)Nc2ccccc2N1c1ccccc1] |
| (32) | 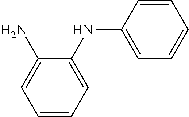 [SMILES: Nc1ccccc1Nc1ccccc1] |
| (33) | 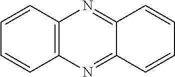 [SMILES: c1ccc2nc3ccccc3nc2c1] |
| (34) | 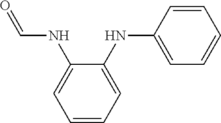 [SMILES: O=CNc1ccccc1Nc1ccccc1] |
| (35) | 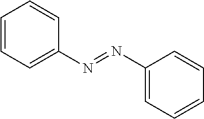 [SMILES: c1ccc(/N=N/c2ccccc2)cc1] |
| (36) | 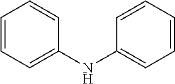 [SMILES: c1ccc(Nc2ccccc2)cc1] |
| (37) | 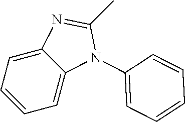 [SMILES: Cc1nc2ccccc2n1-c1ccccc1] |
| (38) | 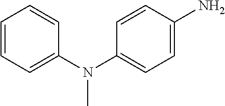 |
| (39) | 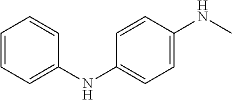 |
| (40) | 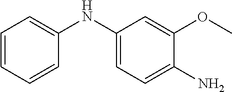 [SMILES: COc1cc(Nc2ccccc2)ccc1N] |
| (41) | 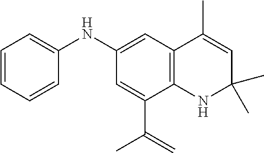 [SMILES: C=C(C)c1cc(Nc2ccccc2)cc2c1NC(C)(C)C=C2C] |
| (42) | 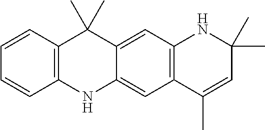 [SMILES: CC1=CC(C)(C)Nc2cc3c(cc21)Nc1ccccc1C3(C)C] |
| (43) | 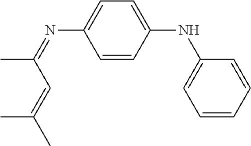 [SMILES: CC(C)=C/C(C)=N\c1ccc(Nc2ccccc2)cc1] |
| (44) | 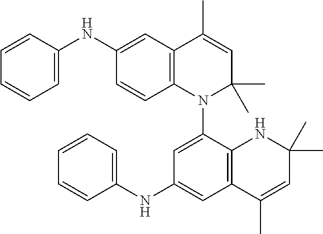 [SMILES: CC1=CC(C)(C)N(c2cc(Nc3ccccc3)cc3c2NC(C)(C)C=C3C)c2ccc(Nc3ccccc3)cc21] |
| (45) | 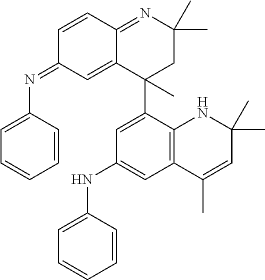 [SMILES: CC1=CC(C)(C)Nc2c1cc(Nc1ccccc1)cc2C1(C)CC(C)(C)N=C2C=C/C(=N/c3ccccc3)C=C21] |
| (46) | 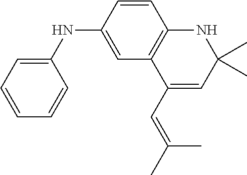 [SMILES: CC(C)=CC1=CC(C)(C)Nc2ccc(Nc3ccccc3)cc21] |
| (47) | 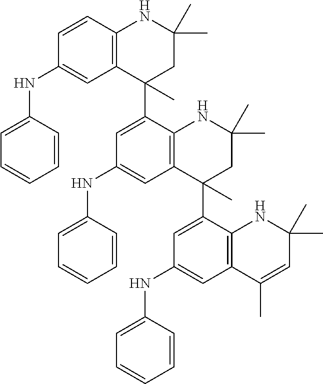 [SMILES: CC1=CC(C)(C)Nc2c1cc(Nc1ccccc1)cc2C1(C)CC(C)(C)Nc2c(C3(C)CC(C)(C)Nc4ccc(Nc5ccccc5)cc43)cc(Nc3ccccc3)cc21] |
| (48) | 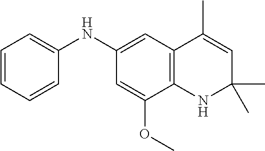 [SMILES: COc1cc(Nc2ccccc2)cc2c1NC(C)(C)C=C2C] |
| (49) | 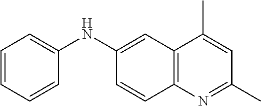 [SMILES: Cc1cc(C)c2cc(Nc3ccccc3)ccc2n1] |
| (50) | 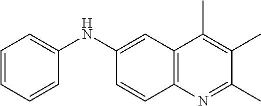 [SMILES: Cc1nc2ccc(Nc3ccccc3)cc2c(C)c1C] |

The compounds in this Section I are collectively referred to as “Compounds of the Disclosure” and individually as a “Compound of the Disclosure.”

### II. Compositions of the Disclosure

In another embodiment, the present disclosure provides a composition comprising a Compound of the Disclosure.

In another embodiment, the present disclosure provides a composition comprising a compound having Formula (I). In another embodiment, the composition comprises 0.0001% to 99.9% of Formula (I) as measured by high performance liquid chromatography (HPLC) analysis, gas chromatography (GC) analysis, and/or a combination of HPLC and GC chromatography analysis.

In another embodiment, the present disclosure provides a composition comprising Compound (1). In another embodiment, the composition comprises about 0.000001% to 99.9% of Compound (1) as measured by HPLC and/or GC analysis. In another embodiment, the composition comprises about 0.0001% to 99.9% of Compound (1) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (1). In another embodiment, the composition comprises about 0.000001% to about 15% of Compound (1) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (1). Compositions comprising Compound (1) may optionally contain any one or more of the other Compounds of the Disclosure listed in Table 1. In one embodiment, compositions comprising Compound (1) also comprise Compound (10) and optionally any one or more of the other Compounds of the Disclosure listed in Table 1.

In another embodiment, the present disclosure provides a composition comprising Compound (2). In another embodiment, the composition comprises about 0.000001% to 99.9% of Compound (2) as measured by HPLC and/or GC analysis. In another embodiment, the composition comprises about 0.0001% to 99.9% of Compound (2) as measured by HPLC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (2). In another embodiment, the composition comprises about 0.000001% to about 15% of Compound (2) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (2). Compositions comprising Compound (2) may optionally contain any one or more of the other Compounds of the Disclosure listed in Table 1. In one embodiment, compositions comprising Compound (2) also comprise Compound (10) and optionally any one or more of the other Compounds of the Disclosure listed in Table 1.

In another embodiment, the present disclosure provides compound having Formula (II), or a salt or solvate thereof:

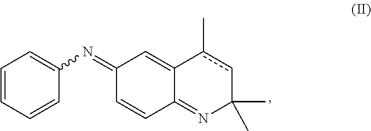

wherein [symbol] represents a single or double bond.

In another embodiment, the disclosure provides compound having Formula (II), wherein [symbol] represents a single bond, i.e., Compound (3), or a salt or solvate thereof:

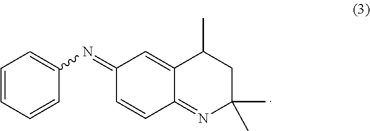 [SMILES: CC1CC(C)(C)N=C2C=CC(=Nc3ccccc3)C=C21]

In another embodiment, the present disclosure provides a compound having Formula (II), wherein [symbol] represents a double bond, i.e., Compound (4), or a salt or solvate thereof:

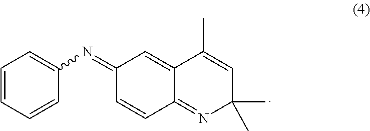 [SMILES: CC1=CC(C)(C)N=C2C=CC(=Nc3ccccc3)C=C12]

In another embodiment, the present disclosure provides a composition comprising a compound having Formula (II), or a salt or solvate thereof. In another embodiment, the composition comprises about 0.0001% to 99.9% of Formula (II) as measured by HPLC analysis.

In another embodiment, the present disclosure provides a composition comprising Compound (3). In another embodiment, the composition comprises about 0.000001% to 99.9% of Compound (3) as measured by HPLC and/or GC analysis. In another embodiment, the composition comprises about 0.0001% to 99.9% of Compound (3) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (3). In another embodiment, the composition comprises about 0.000001% to about 15% of Compound (3) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (3). Compositions comprising Compound (3) may optionally contain any one or more of the other Compounds of the Disclosure listed in Table 1. In one embodiment, compositions comprising Compound (3) also comprise Compound (10) and optionally any one or more of the other Compounds of the Disclosure listed in Table 1.

In another embodiment, the present disclosure provides a composition comprising Compound (4). In another embodiment, the composition comprises about 0.000001% to 99.9% of Compound (4) as measured by HPLC and/or GC analysis. In another embodiment, the composition comprises about 0.0001% to 99.9% of Compound (4) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (4). In another embodiment, the composition comprises about 0.000001% to about 15% of Compound (4) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (4). Compositions comprising Compound (4) may optionally contain any one or more of the other Compounds of the Disclosure listed in Table 1. In one embodiment, compositions comprising Compound (4) also comprise Compound (10) and optionally any one or more of the other Compounds of the Disclosure listed in Table 1.

In another embodiment, the present disclosure provides a composition comprising Compound (4) and Compound (10). In another embodiment, the composition comprises about 50% to 99.9% of Compound (4) as measured by HPLC analysis, e.g., about 75% to 99.9%, about 90% to 99.9%, or about 95% to 99.9% of Compound (4).

In another embodiment, the present disclosure provides a compound having Formula (III), or a salt or solvate thereof:

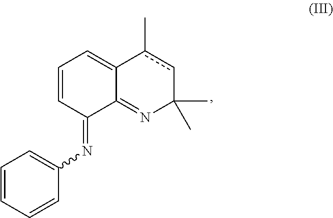

wherein [symbol] represents a single or double bond.

In another embodiment, the present disclosure provides a compound having Formula (III), wherein [symbol] represents a single bond, i.e., Compound (5), or a salt or solvate thereof:

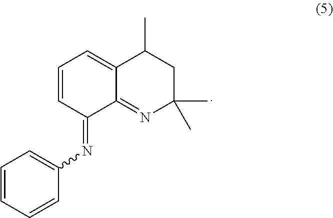 [SMILES: CC1CC(C)(C)N=C2C1=CC=CC2=Nc1ccccc1]

In another embodiment, the present disclosure provides a compound having Formula (III), wherein [symbol] represents a double bond, i.e., Compound (6), or a salt or solvate thereof:

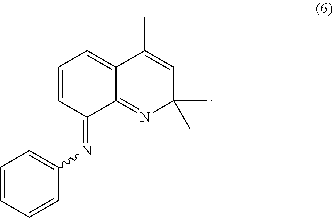 [SMILES: CC1=CC(C)(C)N=C2C1=CC=CC2=Nc1ccccc1]

In another embodiment, the present disclosure provides a composition comprising a compound having Formula (III), or a salt or solvate thereof In another embodiment, the composition comprises about 0.0001% to 99.9% of Formula (III) as measured by HPLC analysis.

In another embodiment, the present disclosure provides a composition comprising Compound (5). In another embodiment, the composition comprises about 0.000001% to 99.9% of Compound (5) as measured by HPLC and/or GC analysis. In another embodiment, the composition comprises about 0.0001% to 99.9% of Compound (5) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (5). In another embodiment, the composition comprises about 0.000001% to about 15% of Compound (5) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (5). Compositions comprising Compound (5) may optionally contain any one or more of the other Compounds of the Disclosure listed in Table 1. In one embodiment, compositions comprising Compound (5) also comprise Compound (10) and optionally any one or more of the other Compounds of the Disclosure listed in Table 1.

In another embodiment, the present disclosure provides a composition comprising Compound (6). In another embodiment, the composition comprises about 0.000001% to 99.9% of Compound (6) as measured by HPLC and/or GC analysis. In another embodiment, the composition comprises about 0.0001% to 99.9% of Compound (6) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (6). In another embodiment, the composition comprises about 0.000001% to about 15% of Compound (6) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (6). Compositions comprising Compound (6) may optionally contain any one or more of the other Compounds of the Disclosure listed in Table 1. In one embodiment, compositions comprising Compound (6) also comprise Compound (10) and optionally any one or more of the other Compounds of the Disclosure listed in Table 1.

In another embodiment, the present disclosure provides a composition comprising Compound (7). In another embodiment, the composition comprises about 0.000001% to 99.9% of Compound (7) as measured by HPLC and/or GC analysis. In another embodiment, the composition comprises about 0.0001% to 99.9% of Compound (7) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (7). In another embodiment, the composition comprises about 0.000001% to about 15% of Compound (7) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (7). Compositions comprising Compound (7) may optionally contain any one or more of the other Compounds of the Disclosure listed in Table 1. In one embodiment, compositions comprising Compound (7) also comprise Compound (10) and optionally any one or more of the other Compounds of the Disclosure listed in Table 1.

In another embodiment, the present disclosure provides a composition comprising Compound (8). In another embodiment, the composition comprises about 0.000001% to 99.9% of Compound (8) as measured by HPLC and/or GC analysis. In another embodiment, the composition comprises about 0.0001% to 99.9% of Compound (8) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (8). In another embodiment, the composition comprises about 0.000001% to about 15% of Compound (8) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (8). Compositions comprising Compound (8) may optionally contain any one or more of the other Compounds of the Disclosure listed in Table 1. In one embodiment, compositions comprising Compound (8) also comprise Compound (10) and optionally any one or more of the other Compounds of the Disclosure listed in Table 1.

In another embodiment, the present disclosure provides a composition comprising a compound having Formula (IV):

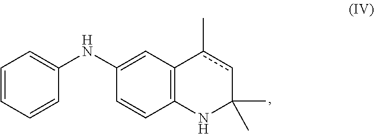

or a salt or solvate thereof, wherein [symbol] represents a single or double bond. In another embodiment, the composition comprises about 50% to 99.9% of Formula (IV) as measured by HPLC analysis, e.g., about 75% to 99.9%, about 80% to 99.9%, about 85% to 99.9%, about 90% to 99.9%, or about 95% to 99.9% of Formula (IV).

In another embodiment, the present disclosure provides a composition comprising a compound having Formula (IV), wherein [symbol] represents a single bond, i.e., Compound (9), or a salt or solvate thereof:

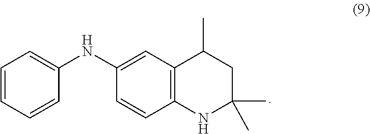 [SMILES: CC1CC(C)(C)Nc2ccc(Nc3ccccc3)cc21]

In another embodiment, the composition comprises about 25% to 99.9% of Compound (9) as measured by HPLC and/or GC analysis. In another embodiment, the composition comprises about 50% to 99.9% of Compound (9) as measured by HPLC and/or GC analysis, e.g., about 75% to 99.9%, about 80% to 99.9%, about 85% to 99.9%, about 90% to 99.9%, or about 95% to 99.9% of Compound (9).

In another embodiment, the present disclosure provides a composition comprising a compound having Formula (IV), wherein [symbol] represents a double bond, i.e., Compound (10), or a salt or solvate thereof:

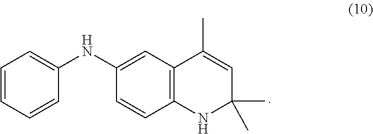 [SMILES: CC1=CC(C)(C)Nc2ccc(Nc3ccccc3)cc21]

In another embodiment, the composition comprises about 25% to 99.9% of Compound (10) as measured by HPLC and/or GC analysis. In another embodiment, the composition comprises about 50% to 99.9% of Compound (10) as measured by HPLC and/or analysis, e.g., about 75% to 99.9%, about 80% to 99.9%, about 85% to 99.9%, about 90% to 99.9%, or about 55% to 99.9% of Compound (10). In another embodiment, the composition comprises about 25%, about 30%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% of Compound (10). Compositions comprising Compound (10) may optionally contain any one or more of the other Compounds of the Disclosure listed in Table 1. Such compositions may comprise about 0.000001% to about 15% of the one or more other Compounds of the Disclosure as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of the one or more other Compounds of the Disclosure.

In another embodiment, the present disclosure provides a composition comprising Compound (11A). In another embodiment, the composition comprises about 0.000001% to 99.9% of Compound (11A) as measured by HPLC and/or GC analysis. In another embodiment, the composition comprises about 0.0001% to 99.9% of Compound (11A) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (11A). In another embodiment, the composition comprises about 0.000001% to about 15% of Compound (11A) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (11A). Compositions comprising Compound (11A) may optionally contain any one or more of the other Compounds of the Disclosure listed in Table 1. In one embodiment, compositions comprising Compound (11A) also comprise Compound (10) and optionally any one or more of the other Compounds of the Disclosure listed in Table 1.

In another embodiment, the present disclosure provides a composition comprising Compound (11B). In another embodiment, the composition comprises about 0.000001% to 99.9% of Compound (111B) as measured by HPLC and/or GC analysis. In another embodiment, the composition comprises about 0.0001% to 99.9% of Compound (111B) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (11B). In another embodiment, the composition comprises about 0.000001% to about 15% of Compound (11B) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (11B). Compositions comprising Compound (11B) may optionally contain any one or more of the other Compounds of the Disclosure listed in Table 1. In one embodiment, compositions comprising Compound (11B) also comprise Compound (10) and optionally any one or more of the other Compounds of the Disclosure listed in Table 1.

In another embodiment, the present disclosure provides a composition comprising Compound (12). In another embodiment, the composition comprises about 0.000001% to 99.9% of Compound (12) as measured by HPLC and/or GC analysis. In another embodiment, the composition comprises about 0.0001% to 99.9% of Compound (12) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (12). In another embodiment, the composition comprises about 0.000001% to about 15% of Compound (12) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (12). Compositions comprising Compound (12) may optionally contain any one or more of the other Compounds of the Disclosure listed in Table 1. In one embodiment, compositions comprising Compound (12) also comprise Compound (10) and optionally any one or more of the other Compounds of the Disclosure listed in Table 1.

In another embodiment, the present disclosure provides a composition comprising Compound (13). In another embodiment, the composition comprises about 0.000001% to 99.9% of Compound (13) as measured by HPLC and/or GC analysis. In another embodiment, the composition comprises about 0.0001% to 99.9% of Compound (13) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (13). In another embodiment, the composition comprises about 0.000001% to about 15% of Compound (13) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (13). Compositions comprising Compound (13) may optionally contain any one or more of the other Compounds of the Disclosure listed in Table 1. In one embodiment, compositions comprising Compound (13) also comprise Compound (10) and optionally any one or more of the other Compounds of the Disclosure listed in Table 1.

In another embodiment, the present disclosure provides a composition comprising Compound (14). In another embodiment, the composition comprises about 0.000001% to 99.9% of Compound (14) as measured by HPLC and/or GC analysis. In another embodiment, the composition comprises about 0.0001% to 99.9% of Compound (14) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (14). In another embodiment, the composition comprises about 0.000001% to about 15% of Compound (14) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (14). Compositions comprising Compound (14) may optionally contain any one or more of the other Compounds of the Disclosure listed in Table 1. In one embodiment, compositions comprising Compound (14) also comprise Compound (10) and optionally any one or more of the other Compounds of the Disclosure listed in Table 1.

In another embodiment, the present disclosure provides a composition comprising Compound (15). In another embodiment, the composition comprises about 0.000001% to 99.9% of Compound (15) as measured by HPLC and/or GC analysis. In another embodiment, the composition comprises about 0.0001% to 99.9% of Compound (15) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (15). In another embodiment, the composition comprises about 0.000001% to about 15% of Compound (15) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (15). Compositions comprising Compound (15) may optionally contain any one or more of the other Compounds of the Disclosure listed in Table 1. In one embodiment, compositions comprising Compound (15) also comprise Compound (10) and optionally any one or more of the other Compounds of the Disclosure listed in Table 1.

In another embodiment, the present disclosure provides a composition comprising Compound (16). In another embodiment, the composition comprises about 0.000001% to 99.9% of Compound (16) as measured by HPLC and/or GC analysis. In another embodiment, the composition comprises about 0.0001% to 99.9% of Compound (16) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (16). In another embodiment, the composition comprises about 0.000001% to about 15% of Compound (16) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (16). Compositions comprising Compound (16) may optionally contain any one or more of the other Compounds of the Disclosure listed in Table 1. In one embodiment, compositions comprising Compound (16) also comprise Compound (10) and optionally any one or more of the other Compounds of the Disclosure listed in Table 1.

In another embodiment, the present disclosure provides a composition comprising Compound (17). In another embodiment, the composition comprises about 0.000001% to 99.9% of Compound (17) as measured by HPLC and/or GC analysis. In another embodiment, the composition comprises about 0.0001% to 99.9% of Compound (17) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (17). In another embodiment, the composition comprises about 0.000001% to about 15% of Compound (17) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (17). Compositions comprising Compound (17) may optionally contain any one or more of the other Compounds of the Disclosure listed in Table 1. In one embodiment, compositions comprising Compound (17) also comprise Compound (10) and optionally any one or more of the other Compounds of the Disclosure listed in Table 1.

In another embodiment, the present disclosure provides a composition comprising Compound (18). In another embodiment, the composition comprises about 0.000001% to 99.9% of Compound (18) as measured by HPLC and/or GC analysis. In another embodiment, the composition comprises about 0.0001% to 99.9% of Compound (18) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (118). In another embodiment, the composition comprises about 0.000001% to about 15% of Compound (18) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (18). Compositions comprising Compound (18) may optionally contain any one or more of the other Compounds of the Disclosure listed in Table 1. In one embodiment, compositions comprising Compound (18) also comprise Compound (10) and optionally any one or more of the other Compounds of the Disclosure listed in Table 1.

In another embodiment, the present disclosure provides a composition comprising Compound (19). In another embodiment, the composition comprises about 0.000001% to 99.9% of Compound (19) as measured by HPLC and/or GC analysis. In another embodiment, the composition comprises about 0.0001% to 99.9% of Compound (19) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (19). In another embodiment, the composition comprises about 0.000001% to about 15% of Compound (19) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (19). Compositions comprising Compound (19) may optionally contain any one or more of the other Compounds of the Disclosure listed in Table 1. In one embodiment, compositions comprising Compound (19) also comprise Compound (10) and optionally any one or more of the other Compounds of the Disclosure listed in Table 1.

In another embodiment, the present disclosure provides a composition comprising Compound (20A). In another embodiment, the composition comprises about 0.000001% to 99.9% of Compound (20A) as measured by HPLC and/or GC analysis. In another embodiment, the composition comprises about 0.0001% to 99.9% of Compound (20A) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (20A). In another embodiment, the composition comprises about 0.000001% to about 15% of Compound (20A) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (20A). Compositions comprising Compound (20A) may optionally contain any one or more of the other Compounds of the Disclosure listed in Table 1. In one embodiment, compositions comprising Compound (20A) also comprise Compound (10) and optionally any one or more of the other Compounds of the Disclosure listed in Table 1.

In another embodiment, the present disclosure provides a composition comprising Compound (20B). In another embodiment, the composition comprises about 0.000001% to 99.9% of Compound (20B) as measured by HPLC and/or GC analysis. In another embodiment, the composition comprises about 0.0001% to 99.9% of Compound (20B) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (20B). In another embodiment, the composition comprises about 0.000001% to about 15% of Compound (20B) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (20B). Compositions comprising Compound (20B) may optionally contain any one or more of the other Compounds of the Disclosure listed in Table 1. In one embodiment, compositions comprising Compound (20B) also comprise Compound (10) and optionally any one or more of the other Compounds of the Disclosure listed in Table 1.

In another embodiment, the present disclosure provides a composition comprising Compound (21). In another embodiment, the composition comprises about 0.000001% to 99.9% of Compound (21) as measured by HPLC and/or GC analysis. In another embodiment, the composition comprises about 0.0001% to 99.9% of Compound (21) as measured by HPLC and/or analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (21). In another embodiment, the composition comprises about 0.000001% to about 15% of Compound (21) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (21). Compositions comprising Compound (21) may optionally contain any one or more of the other Compounds of the Disclosure listed in Table 1. In one embodiment, compositions comprising Compound (21) also comprise Compound (10) and optionally any one or more of the other Compounds of the Disclosure listed in Table 1.

In another embodiment, the present disclosure provides a composition comprising Compound (22). In another embodiment, the composition comprises about 0.000001% to 99.9% of Compound (22) as measured by HPLC and/or GC analysis. In another embodiment, the composition comprises about 0.0001% to 99.9% of Compound (22) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (22). In another embodiment, the composition comprises about 0.000001% to about 15% of Compound (22) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (22). Compositions comprising Compound (22) may optionally contain any one or more of the other Compounds of the Disclosure listed in Table 1. In one embodiment, compositions comprising Compound (22) also comprise Compound (10) and optionally any one or more of the other Compounds of the Disclosure listed in Table 1.

In another embodiment, the present disclosure provides a composition comprising Compound (23). In another embodiment, the composition comprises about 0.000001% to 99.9% of Compound (23) as measured by HPLC and/or GC analysis. In another embodiment, the composition comprises about 50% to 99.9% of Compound (23) as measured by HPLC and/or GC analysis, e.g., about 75% to 99.9%, about 80% to 99.9%, about 85% to 99.9%, about 90% to 99.9%, or about 55% to 99.9% of Compound (23). In another embodiment, the composition comprises about 0.000001% to about 15% of Compound (23) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (23). Compositions comprising Compound (23) may optionally contain any one or more of the other Compounds of the Disclosure listed in Table 1. In one embodiment, compositions comprising Compound (23) also comprise Compound (10) and optionally any one or more of the other Compounds of the Disclosure listed in Table 1.

In another embodiment, the present disclosure provides a composition comprising Compound (24). In another embodiment, the composition comprises about 0.000001% to 99.9% of Compound (24) as measured by HPLC and/or GC analysis. In another embodiment, the composition comprises about 50% to 99.9% of Compound (24) as measured by HPLC analysis, e.g., about 75% to 99.9%, about 80% to 99.9%, about 85% to 99.9%, about 90% to 99.9%, or about 55% to 99.9% of Compound (24). In another embodiment, the composition comprises about 0.000001% to about 15% of Compound (24) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (24). Compositions comprising Compound (24) may optionally contain any one or more of the other Compounds of the Disclosure listed in Table 1. In one embodiment, compositions comprising Compound (24) also comprise Compound (10) and optionally any one or more of the other Compounds of the Disclosure listed in Table 1.

In another embodiment, the present disclosure provides a composition comprising Compound (25). In another embodiment, the composition comprises about 0.000001% to 99.9% of Compound (25) as measured by HPLC and/or GC analysis. In another embodiment, the composition comprises about 0.0001% to 99.9% of Compound (25) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (25). In another embodiment, the composition comprises about 0.000001% to about 15% of Compound (25) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (25). Compositions comprising Compound (25) may optionally contain any one or more of the other Compounds of the Disclosure listed in Table 1. In one embodiment, compositions comprising Compound (25) also comprise Compound (10) and optionally any one or more of the other Compounds of the Disclosure listed in Table 1.

In another embodiment, the present disclosure provides a composition comprising Compound (26). In another embodiment, the composition comprises about 0.000001% to 99.9% of Compound (26) as measured by HPLC and/or GC analysis. In another embodiment, the composition comprises about 0.0001% to 99.9% of Compound (26) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (26). In another embodiment, the composition comprises about 0.000001% to about 15% of Compound (26) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (26). Compositions comprising Compound (26) may optionally contain any one or more of the other Compounds of the Disclosure listed in Table 1. In one embodiment, compositions comprising Compound (26) also comprise Compound (10) and optionally any one or more of the other Compounds of the Disclosure listed in Table 1.

In another embodiment, the present disclosure provides a composition comprising Compound (27A). In another embodiment, the composition comprises about 0.000001% to 99.9% of Compound (27A) as measured by HPLC and/or GC analysis. In another embodiment, the composition comprises about 0.0001% to 99.9% of Compound (27A) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (27A). In another embodiment, the composition comprises about 0.000001% to about 15% of Compound (27A) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (27A). Compositions comprising Compound (27A) may optionally contain any one or more of the other Compounds of the Disclosure listed in Table 1. In one embodiment, compositions comprising Compound (27A) also comprise Compound (10) and optionally any one or more of the other Compounds of the Disclosure listed in Table 1.

In another embodiment, the present disclosure provides a composition comprising Compound (27B). In another embodiment, the composition comprises about 0.000001% to 99.9% of Compound (27B) as measured by HPLC and/or GC analysis. In another embodiment, the composition comprises about 0.0001% to 99.9% of Compound (27B) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (27B). In another embodiment, the composition comprises about 0.000001% to about 15% of Compound (27B) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (27B). Compositions comprising Compound (27B) may optionally contain any one or more of the other Compounds of the Disclosure listed in Table 1. In one embodiment, compositions comprising Compound (27B) also comprise Compound (10) and optionally any one or more of the other Compounds of the Disclosure listed in Table 1.

In another embodiment, the present disclosure provides a composition comprising Compound (28). In another embodiment, the composition comprises about 0.000001% to 99.9% of Compound (28) as measured by HPLC and/or GC analysis. In another embodiment, the composition comprises about 0.0001% to 99.9% of Compound (28) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (28). In another embodiment, the composition comprises about 0.000001% to about 15% of Compound (28) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (28). Compositions comprising Compound (28) may optionally contain any one or more of the other Compounds of the Disclosure listed in Table 1. In one embodiment, compositions comprising Compound (28) also comprise Compound (10) and optionally any one or more of the other Compounds of the Disclosure listed in Table 1.

In another embodiment, the present disclosure provides a composition comprising Compound (29). In another embodiment, the composition comprises about 0.000001% to 99.9% of Compound (29) as measured by HPLC and/or GC analysis. In another embodiment, the composition comprises about 0.0001% to 99.9% of Compound (29) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (29). In another embodiment, the composition comprises about 0.000001% to about 15% of Compound (29) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (29). Compositions comprising Compound (29) may optionally contain any one or more of the other Compounds of the Disclosure listed in Table 1. In one embodiment, compositions comprising Compound (29) also comprise Compound (10) and optionally any one or more of the other Compounds of the Disclosure listed in Table 1.

In another embodiment, the present disclosure provides a composition comprising Compound (30). In another embodiment, the composition comprises about 0.000001% to 99.9% of Compound (30) as measured by HPLC and/or GC analysis. In another embodiment, the composition comprises about 0.0001% to 99.9% of Compound (30) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (30). In another embodiment, the composition comprises about 0.000001% to about 15% of Compound (30) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (30). Compositions comprising Compound (30) may optionally contain any one or more of the other Compounds of the Disclosure listed in Table 1. In one embodiment, compositions comprising Compound (30) also comprise Compound (10) and optionally any one or more of the other Compounds of the Disclosure listed in Table 1.

In another embodiment, the present disclosure provides a composition comprising Compound (31). In another embodiment, the composition comprises about 0.000001% to 99.9% of Compound (31) as measured by HPLC and/or GC analysis. In another embodiment, the composition comprises about 0.0001% to 99.9% of Compound (31) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (31). In another embodiment, the composition comprises about 0.000001% to about 15% of Compound (31) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (31). Compositions comprising Compound (31) may optionally contain any one or more of the other Compounds of the Disclosure listed in Table 1. In one embodiment, compositions comprising Compound (31) also comprise Compound (10) and optionally any one or more of the other Compounds of the Disclosure listed in Table 1.

In another embodiment, the present disclosure provides a composition comprising Compound (32). In another embodiment, the composition comprises about 0.000001% to 99.9% of Compound (32) as measured by HPLC and/or GC analysis. In another embodiment, the composition comprises about 0.0001% to 99.9% of Compound (32) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (32). In another embodiment, the composition comprises about 0.000001% to about 15% of Compound (32) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (32). Compositions comprising Compound (32) may optionally contain any one or more of the other Compounds of the Disclosure listed in Table 1. In one embodiment, compositions comprising Compound (32) also comprise Compound (10) and optionally any one or more of the other Compounds of the Disclosure listed in Table 1.

In another embodiment, the present disclosure provides a composition comprising Compound (33). In another embodiment, the composition comprises about 0.000001% to 99.9% of Compound (33) as measured by HPLC and/or GC analysis. In another embodiment, the composition comprises about 0.0001% to 99.9% of Compound (33) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (33). In another embodiment, the composition comprises about 0.000001% to about 15% of Compound (33) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (33). Compositions comprising Compound (33) may optionally contain any one or more of the other Compounds of the Disclosure listed in Table 1. In one embodiment, compositions comprising Compound (33) also comprise Compound (10) and optionally any one or more of the other Compounds of the Disclosure listed in Table 1.

In another embodiment, the present disclosure provides a composition comprising Compound (34). In another embodiment, the composition comprises about 0.000001% to 99.9% of Compound (34) as measured by HPLC and/or GC analysis. In another embodiment, the composition comprises about 0.0001% to 99.9% of Compound (34) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (34). In another embodiment, the composition comprises about 0.000001% to about 15% of Compound (34) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (34). Compositions comprising Compound (34) may optionally contain any one or more of the other Compounds of the Disclosure listed in Table 1. In one embodiment, compositions comprising Compound (34) also comprise Compound (10) and optionally any one or more of the other Compounds of the Disclosure listed in Table 1.

In another embodiment, the present disclosure provides a composition comprising Compound (35). In another embodiment, the composition comprises about 0.000001% to 99.9% of Compound (35) as measured by HPLC and/or GC analysis. In another embodiment, the composition comprises about 0.0001% to 99.9% of Compound (35) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (35). In another embodiment, the composition comprises about 0.000001% to about 15% of Compound (35) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (35). Compositions comprising Compound (35) may optionally contain any one or more of the other Compounds of the Disclosure listed in Table 1. In one embodiment, compositions comprising Compound (35) also comprise Compound (10) and optionally any one or more of the other Compounds of the Disclosure listed in Table 1.

In another embodiment, the present disclosure provides a composition comprising Compound (36). In another embodiment, the composition comprises about 0.000001% to 99.9% of Compound (36) as measured by HPLC and/or GC analysis. In another embodiment, the composition comprises about 0.0001% to 99.9% of Compound (36) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (36). In another embodiment, the composition comprises about 0.000001% to about 15% of Compound (36) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (36). Compositions comprising Compound (36) may optionally contain any one or more of the other Compounds of the Disclosure listed in Table 1. In one embodiment, compositions comprising Compound (36) also comprise Compound (10) and optionally any one or more of the other Compounds of the Disclosure listed in Table 1.

In another embodiment, the present disclosure provides a composition comprising Compound (37). In another embodiment, the composition comprises about 0.000001% to 99.9% of Compound (37) as measured by HPLC and/or GC analysis. In another embodiment, the composition comprises about 0.0001% to 99.9% of Compound (37) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (37). In another embodiment, the composition comprises about 0.000001% to about 15% of Compound (37) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (37). Compositions comprising Compound (37) may optionally contain any one or more of the other Compounds of the Disclosure listed in Table 1. In one embodiment, compositions comprising Compound (37) also comprise Compound (10) and optionally any one or more of the other Compounds of the Disclosure listed in Table 1.

In another embodiment, the present disclosure provides a composition comprising Compound (38). In another embodiment, the composition comprises about 0.000001% to 99.9% of Compound (38) as measured by HPLC and/or GC analysis. In another embodiment, the composition comprises about 0.0001% to 99.9% of Compound (38) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (38). In another embodiment, the composition comprises about 0.000001% to about 15% of Compound (38) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (38). Compositions comprising Compound (38) may optionally contain any one or more of the other Compounds of the Disclosure listed in Table 1. In one embodiment, compositions comprising Compound (38) also comprise Compound (10) and optionally any one or more of the other Compounds of the Disclosure listed in Table 1.

In another embodiment, the present disclosure provides a composition comprising Compound (39). In another embodiment, the composition comprises about 0.000001% to 99.9% of Compound (39) as measured by HPLC and/or GC analysis. In another embodiment, the composition comprises about 0.0001% to 99.9% of Compound (39) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (39). In another embodiment, the composition comprises about 0.000001% to about 15% of Compound (39) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (39). Compositions comprising Compound (39) may optionally contain any one or more of the other Compounds of the Disclosure listed in Table 1. In one embodiment, compositions comprising Compound (39) also comprise Compound (10) and optionally any one or more of the other Compounds of the Disclosure listed in Table 1.

In another embodiment, the present disclosure provides a composition comprising Compound (40). In another embodiment, the composition comprises about 0.000001% to 99.9% of Compound (40) as measured by HPLC and/or GC analysis. In another embodiment, the composition comprises about 0.0001% to 99.9% of Compound (40) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (40). In another embodiment, the composition comprises about 0.000001% to about 15% of Compound (40) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (40). Compositions comprising Compound (0) may optionally contain any one or more of the other Compounds of the Disclosure listed in Table 1. In one embodiment, compositions comprising Compound (40) also comprise Compound (10) and optionally any one or more of the other Compounds of the Disclosure listed in Table 1.

In another embodiment, the present disclosure provides a composition comprising Compound (41). In another embodiment, the composition comprises about 0.000001% to 99.9% of Compound (41) as measured by HPLC and/or GC analysis. In another embodiment, the composition comprises about 0.0001% to 99.9% of Compound (41) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (41). In another embodiment, the composition comprises about 0.000001% to about 15% of Compound (41) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (41). Compositions comprising Compound (41) may optionally contain any one or more of the other Compounds of the Disclosure listed in Table 1. In one embodiment, compositions comprising Compound (41) also comprise Compound (10) and optionally any one or more of the other Compounds of the Disclosure listed in Table 1.

In another embodiment, the present disclosure provides a composition comprising Compound (42). In another embodiment, the composition comprises about 0.000001% to 99.9% of Compound (42) as measured by HPLC and/or GC analysis. In another embodiment, the composition comprises about 0.0001% to 99.9% of Compound (42) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (42). In another embodiment, the composition comprises about 0.000001% to about 15% of Compound (42) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (42). Compositions comprising Compound (42) may optionally contain any one or more of the other Compounds of the Disclosure listed in Table 1. In one embodiment, compositions comprising Compound (42) also comprise Compound (10) and optionally any one or more of the other Compounds of the Disclosure listed in Table 1.

In another embodiment, the present disclosure provides a composition comprising Compound (43). In another embodiment, the composition comprises about 0.000001% to 99.9% of Compound (43) as measured by HPLC and/or GC analysis. In another embodiment, the composition comprises about 0.0001% to 99.9% of Compound (43) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (43). In another embodiment, the composition comprises about 0.000001% to about 15% of Compound (43) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (43). Compositions comprising Compound (43) may optionally contain any one or more of the other Compounds of the Disclosure listed in Table 1. In one embodiment, compositions comprising Compound (43) also comprise Compound (10) and optionally any one or more of the other Compounds of the Disclosure listed in Table 1.

In another embodiment, the present disclosure provides a composition comprising Compound (44). In another embodiment, the composition comprises about 0.000001% to 99.9% of Compound (44) as measured by HPLC and/or GC analysis. In another embodiment, the composition comprises about 0.0001% to 99.9% of Compound (44) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (44). In another embodiment, the composition comprises about 0.000001% to about 15% of Compound (44) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (44). Compositions comprising Compound (44) may optionally contain any one or more of the other Compounds of the Disclosure listed in Table 1. In one embodiment, compositions comprising Compound (44) also comprise Compound (10) and optionally any one or more of the other Compounds of the Disclosure listed in Table 1.

In another embodiment, the present disclosure provides a composition comprising Compound (45). In another embodiment, the composition comprises about 0.000001% to 99.9% of Compound (45) as measured by HPLC and/or GC analysis. In another embodiment, the composition comprises about 0.0001% to 99.9% of Compound (45) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (45). In another embodiment, the composition comprises about 0.000001% to about 15% of Compound (45) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (45). Compositions comprising Compound (45) may optionally contain any one or more of the other Compounds of the Disclosure listed in Table 1. In one embodiment, compositions comprising Compound (45) also comprise Compound (10) and optionally any one or more of the other Compounds of the Disclosure listed in Table 1.

In another embodiment, the present disclosure provides a composition comprising Compound (46). In another embodiment, the composition comprises about 0.000001% to 99.9% of Compound (46) as measured by HPLC and/or GC analysis. In another embodiment, the composition comprises about 0.0001% to 99.9% of Compound (46) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (46). In another embodiment, the composition comprises about 0.000001% to about 15% of Compound (46) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (46). Compositions comprising Compound (46) may optionally contain any one or more of the other Compounds of the Disclosure listed in Table 1. In one embodiment, compositions comprising Compound (46) also comprise Compound (10) and optionally any one or more of the other Compounds of the Disclosure listed in Table 1.

In another embodiment, the present disclosure provides a composition comprising Compound (47). In another embodiment, the composition comprises about 0.000001% to 99.9% of Compound (47) as measured by HPLC and/or GC analysis. In another embodiment, the composition comprises about 0.0001% to 99.9% of Compound (47) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (47). In another embodiment, the composition comprises about 0.000001% to about 15% of Compound (47) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (47). Compositions comprising Compound (47) may optionally contain any one or more of the other Compounds of the Disclosure listed in Table 1. In one embodiment, compositions comprising Compound (47) also comprise Compound (10) and optionally any one or more of the other Compounds of the Disclosure listed in Table 1.

In another embodiment, the present disclosure provides a composition comprising Compound (48). In another embodiment, the composition comprises about 0.000001% to 99.9% of Compound (48) as measured by HPLC and/or GC analysis. In another embodiment, the composition comprises about 0.0001% to 99.9% of Compound (48) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (48). In another embodiment, the composition comprises about 0.000001% to about 15% of Compound (48) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (48). Compositions comprising Compound (48) may optionally contain any one or more of the other Compounds of the Disclosure listed in Table 1. In one embodiment, compositions comprising Compound (48) also comprise Compound (10) and optionally any one or more of the other Compounds of the Disclosure listed in Table 1.

In another embodiment, the present disclosure provides a composition comprising Compound (49). In another embodiment, the composition comprises about 0.000001% to 99.9% of Compound (49) as measured by HPLC and/or GC analysis. In another embodiment, the composition comprises about 0.0001% to 99.9% of Compound (49) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (49). In another embodiment, the composition comprises about 0.000001% to about 15% of Compound (49) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (49). Compositions comprising Compound (49) may optionally contain any one or more of the other Compounds of the Disclosure listed in Table 1. In one embodiment, compositions comprising Compound (49) also comprise Compound (10) and optionally any one or more of the other Compounds of the Disclosure listed in Table 1.

In another embodiment, the present disclosure provides a composition comprising Compound (50). In another embodiment, the composition comprises about 0.000001% to 99.9% of Compound (50) as measured by HPLC and/or GC analysis. In another embodiment, the composition comprises about 0.0001% to 99.9% of Compound (50) as measured by HPLC and/or GC analysis, e.g., about 0.0001% to about 50%, about 0.0001% to about 25%, about 0.0001% to about 10%, about 0.0001% to about 5%, about 0.0001% to about 1%, about 50% to 99.9%, about 75% to 99.9%, about 90% to 99.9%, or about 95% to 99.9% of Compound (50). In another embodiment, the composition comprises about 0.000001% to about 15% of Compound (50) as measured by HPLC and/or GC analysis, e.g., about 0.000001% to about 10%, about 0.000001% to about 5%, about 0.000001% to about 3%, about 0.000001% to about 1%, about 0.00001% to about 3%, about 0.00001% to about 1%, about 0.0001% to about 3%, about 0.0001% to about 1%, about 0.001% to about 3%, or about 0.001% to about 1% of Compound (50). Compositions comprising Compound (50) may optionally contain any one or more of the other Compounds of the Disclosure listed in Table 1. In one embodiment, compositions comprising Compound (50) also comprise Compound (10) and optionally any one or more of the other Compounds of the Disclosure listed in Table 1.

In another embodiment, the present disclosure provides a composition comprising a compound having Formula (IV), e.g., Compound (9) or Compound (10), or a salt or solvate thereof; and:
(a) a compound having Formula (I), e.g., Compound (1) or Compound (2), or a salt or solvate thereof:

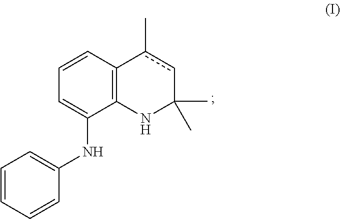

or
(b) a compound having Formula (II), e.g., Compound (3) or Compound (4), or a salt or solvate thereof:

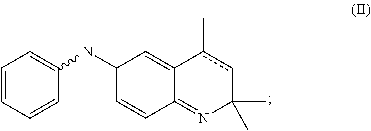

or
(c) a compound having Formula (III), e.g., Compound (5) or Compound (6), or a salt or solvate thereof:

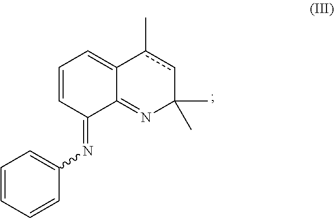

or
(d) Compound (7), or a salt or solvate thereof:

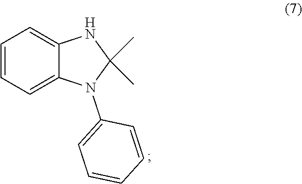 [SMILES: CC1(C)Nc2ccccc2N1c1ccccc1]

or
(e) Compound (8), or a salt or solvate thereof:

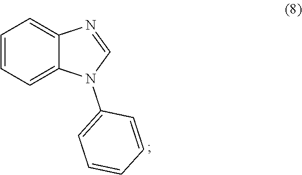 [SMILES: c1ccc(-n2cnc3ccccc32)cc1]

or
(f) Compound (11A), or a salt or solvate thereof:

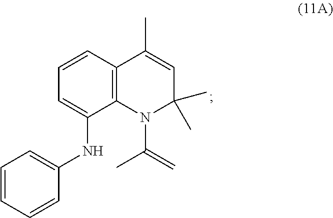 [SMILES: C=C(C)N1c2c(Nc3ccccc3)cccc2C(C)=CC1(C)C]

or
(g) Compound (11B), or a salt or solvate thereof:

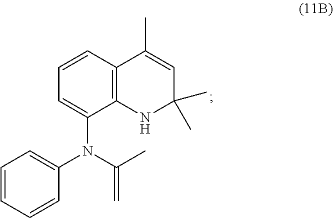 [SMILES: C=C(C)N(c1ccccc1)c1cccc2c1NC(C)(C)C=C2C]

or
(h) Compound (20A), or a salt or solvate thereof:

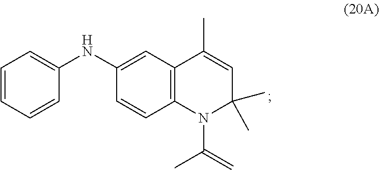 [SMILES: C=C(C)N1c2ccc(Nc3ccccc3)cc2C(C)=CC1(C)C]

or
(i) Compound (20B), or a salt or solvate thereof:

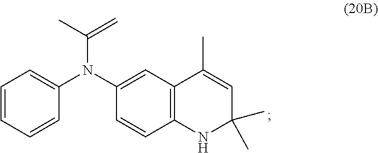 [SMILES: C=C(C)N(c1ccccc1)c1ccc2c(c1)C(C)=CC(C)(C)N2]

or
(i) Compound (21), or a salt or solvate thereof:

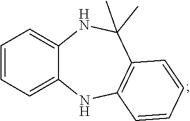 [SMILES: CC1(C)Nc2ccccc2Nc2ccccc21]

or
(k) Compound (22), or a salt or solvate thereof:

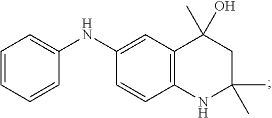 [SMILES: CC1(C)CC(C)(O)c2cc(Nc3ccccc3)ccc2N1]

or

(l) Compound (23), or a salt or solvate thereof:

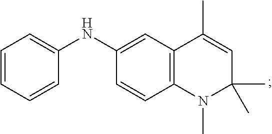 [SMILES: CC1=CC(C)(C)N(C)c2ccc(Nc3ccccc3)cc21]

or
(m) Compound (24), or a salt or solvate thereof:

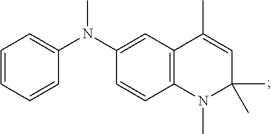 [SMILES: CC1=CC(C)(C)N(C)c2ccc(N(C)c3ccccc3)cc21]

or

(n) Compound (25), or a salt or solvate thereof:

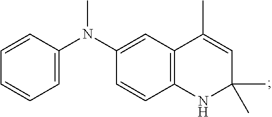 [SMILES: CC1=CC(C)(C)Nc2ccc(N(C)c3ccccc3)cc21]

or

(o) Compound (37), or a salt or solvate thereof:

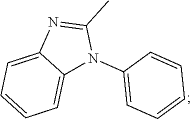 [SMILES: Cc1nc2ccccc2n1-c1ccccc1]

or
(p) a combination of Formula (I), or a salt or solvate thereof; Formula (II), or a salt or solvate thereof; Formula (III), or a salt or solvate thereof, Compound (7), or a salt or solvate thereof, Compound (8), or a salt or solvate thereof, Compound (11A), or a salt or solvate thereof, Compound (11B), or a salt or solvate thereof, Compound (20A), or a salt or solvate thereof, Compound (20B), or a salt or solvate thereof, Compound (21), or a salt or solvate thereof, Compound (22), or a salt or solvate thereof, Compound (23), or a salt or solvate thereof, Compound (24), or a salt or solvate thereof, Compound (25), or a salt or solvate thereof, and/or Compound (37), or a salt or solvate thereof,
wherein [symbol] represents a single or double bond.

In another embodiment, the present disclosure provides a composition comprising a compound having Formula (IV), or a salt or solvate thereof, and a compound having Formula (I), or a salt or solvate thereof In another embodiment, the composition comprises Formula (IV) and about 0.001% to about 10% of Formula (I) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, or about 0.05% to about 0.5% of Formula (I). In another embodiment, the composition comprises Formula (IV) and about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.3%, about 0.4%, about 0.5%, about 0.6%, about 0.7%, about 0.8%, about 0.9%, or about 1% of Formula (I). In another embodiment, the compound having Formula (IV) is Compound (9). In another embodiment, the compound having Formula (IV) is Compound (10). In another embodiment, the compound having Formula (I) is Compound (1). In another embodiment, the compound having Formula (I) is Compound (2).

In another embodiment, the present disclosure provides a composition comprising a compound having Formula (IV), or a salt or solvate thereof, and a compound having Formula (I), or a salt or solvate thereof, wherein the weight ratio of Formula (I) to Formula (IV) is 0.001 to 0.1, e.g., 0.001 to 0.05, 0.001 to 0.01, or 0.001 to 0.005 of Formula (I) to Formula (IV). In another embodiment, the compound having Formula (IV) is Compound (10). In another embodiment, the compound having Formula (I) is Compound (1). In another embodiment, the compound having Formula (I) is Compound (2).

In another embodiment, the present disclosure provides a composition comprising a compound having Formula (IV), or a salt or solvate thereof, and a compound having Formula (II), or a salt or solvate thereof In another embodiment, the composition comprises Formula (IV) and about 0.001% to about 10% of Formula (II) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Formula (II). In another embodiment, the composition comprises Formula (IV) and about 0.01%, about 0.2%, about 0.21%, about 0.22%, about 0.23%, about 0.24%, about 0.25%, about 0.26%, about 0.27%, about 0.28%, about 0.29%, about 0.3%, about 0.31%, about 0.32%, about 0.33%, about 0.34%, about 0.35%, about 0.36%, about 0.37%, about 0.38%, about 0.39%, about 0.4%, about 0.41%, about 0.42%, about 0.43%, about 0.44%, about 0.45%, about 0.46%, about 0.47%, about 0.48%, about 0.49%, about 0.5%, about 0.6%, about 0.7%, about 0.8%, about 0.9%, or about 1%, of Formula (II). In another embodiment, the compound having Formula (IV) is Compound (9). In another embodiment, the compound having Formula (IV) is Compound (10). In another embodiment, the compound having Formula (II) is Compound (3). In another embodiment, the compound having Formula (II) is Compound (4).

In another embodiment, the present disclosure provides a composition comprising a compound having Formula (IV), or a salt or solvate thereof, and a compound having Formula (II), or a salt or solvate thereof, wherein the weight ratio of Formula (II) to Formula (IV) is 0.001 to 0.1, e.g., 0.001 to 0.05, 0.001 to 0.01, or 0.001 to 0.005 of Formula (II) to Formula (IV). In another embodiment, the compound having Formula (IV) is Compound (9). In another embodiment, the compound having Formula (IV) is Compound (10). In another embodiment, the compound having Formula (II) is Compound (3). In another embodiment, the compound having Formula (II) is Compound (4).

In another embodiment, the present disclosure provides a composition comprising a compound having Formula (IV), or a salt or solvate thereof, and a compound having Formula (III), or a salt or solvate thereof. In another embodiment, the composition comprises Formula (IV) and about 0.001% to about 10% of Formula (III) as measured by HPLC analysis, e.g., about 0.01% to about 10%, about 0.1% to about 10%, about 0.001% to about 5%, about 0.01% to about 5%, or about 0.1% to about 5% Formula (III). In another embodiment, the compound having Formula (IV) is Compound (9). In another embodiment, the compound having Formula (IV) is Compound (10). In another embodiment, the compound having Formula (III) is Compound (5). In another embodiment, the compound having Formula (III) is Compound (6).

In another embodiment, the present disclosure provides a composition comprising a compound having Formula (IV), or a salt or solvate thereof, and a compound having Formula (III), or a salt or solvate thereof, wherein the weight ratio of Formula (III) to Formula (IV) is 0.001 to 0.1, e.g., 0.001 to 0.05, 0.001 to 0.01, or 0.001 to 0.005 of Formula (III) to Formula (IV). In another embodiment, the compound having Formula (IV) is Compound (9). In another embodiment, the compound having Formula (IV) is Compound (10). In another embodiment, the compound having Formula (III) is Compound (5). In another embodiment, the compound having Formula (III) is Compound (6).

In another embodiment, the present disclosure provides a composition comprising a compound having Formula (IV), or a salt or solvate thereof, and Compound (7), or a salt or solvate thereof In another embodiment, the composition comprises Formula (IV) and about 0.001% to about 10% of Compound (7) as measured by HPLC analysis, e.g., about 0.01% to about 10%, about 0.1% to about 10%, about 0.001% to about 5%, about 0.01% to about 5%, or about 0.1% to about 5% of Compound (7). In another embodiment, the compound having Formula (IV) is Compound (9). In another embodiment, the compound having Formula (IV) is Compound (10).

In another embodiment, the present disclosure provides a composition comprising a compound having Formula (IV), or a salt or solvate thereof, and Compound (7), or a salt or solvate thereof, wherein the weight ratio of Compound (7) to Formula (IV) is 0.001 to 0.1, e.g., 0.001 to 0.05, 0.001 to 0.01, or 0.001 to 0.005 of Compound (7) to Formula (IV). In another embodiment, the compound having Formula (IV) is Compound (9). In another embodiment, the compound having Formula (IV) is Compound (10).

In another embodiment, the present disclosure provides a composition comprising a compound having Formula (IV), or a salt or solvate thereof, and Compound (8), or a salt or solvate thereof In another embodiment, the composition comprises Formula (IV) and about 0.001% to about 10% of Compound (8) as measured by HPLC analysis, e.g., about 0.01% to about 10%, about 0.1% to about 10%, about 0.001% to about 5%, about 0.01% to about 5%, or about 0.1% to about 5% of Compound (8). In another embodiment, the compound having Formula (IV) is Compound (9). In another embodiment, the compound having Formula (IV) is Compound (10).

In another embodiment, the present disclosure provides a composition comprising a compound having Formula (IV), or a salt or solvate thereof, and Compound (8), or a salt or solvate thereof, wherein the weight ratio of Compound (8) to Formula (IV) is 0.001 to 0.1, e.g., 0.001 to 0.05, 0.001 to 0.01, or 0.001 to 0.005 of Compound (8) to Formula (IV). In another embodiment, the compound having Formula (IV) is Compound (9). In another embodiment, the compound having Formula (IV) is Compound (10).

In another embodiment, the present disclosure provides a composition comprising a compound having Formula (IV), or a salt or solvate thereof, and Compound (11), or a salt or solvate thereof In another embodiment, the composition comprises Formula (IV) and about 0.001% to about 10% of Compound (11) as measured by HPLC analysis, e.g., about 0.01% to about 10%, about 0.1% to about 10%, about 0.001% to about 5%, about 0.01% to about 5%, or about 0.1% to about 5% of Compound (11). In another embodiment, the composition comprises about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.21%, about 0.22%, about 0.23%, about 0.24%, about 0.25%, about 0.26%, about 0.27%, about 0.28%, about 0.29%, about 0.3%, about 0.4%, about 0.5%, about 0.6%, about 0.7%, about 0.8%, about 0.9%, or about 1% of Compound (11). In another embodiment, the compound having Formula (IV) is Compound (9). In another embodiment, the compound having Formula (IV) is Compound (10).

In another embodiment, the present disclosure provides a composition comprising a compound having Formula (IV), or a salt or solvate thereof, and Compound (11A), or a salt or solvate thereof, wherein the weight ratio of Compound (11A) to Formula (IV) is 0.001 to 0.1, e.g., 0.001 to 0.05, 0.001 to 0.01, or 0.001 to 0.005 of Compound (11A) to Formula (IV). In another embodiment, the compound having Formula (IV) is Compound (9). In another embodiment, the compound having Formula (IV) is Compound (10).

In another embodiment, the present disclosure provides a composition comprising a compound having Formula (IV), or a salt or solvate thereof, and Compound (11B), or a salt or solvate thereof, wherein the weight ratio of Compound (111B) to Formula (IV) is 0.001 to 0.1, e.g., 0.001 to 0.05, 0.001 to 0.01, or 0.001 to 0.005 of Compound (11B) to Formula (IV). In another embodiment, the compound having Formula (IV) is Compound (9). In another embodiment, the compound having Formula (IV) is Compound (10).

In another embodiment, the present disclosure provides a composition comprising a compound having Formula (IV), or a salt or solvate thereof, and Compound (20A), or a salt or solvate thereof, wherein the weight ratio of Compound (20A) to Formula (IV) is 0.001 to 0.1, e.g., 0.001 to 0.05, 0.001 to 0.01, or 0.001 to 0.005 of Compound (20A) to Formula (IV). In another embodiment, the compound having Formula (IV) is Compound (9). In another embodiment, the compound having Formula (IV) is Compound (10).

In another embodiment, the present disclosure provides a composition comprising a compound having Formula (IV), or a salt or solvate thereof, and Compound (20B), or a salt or solvate thereof, wherein the weight ratio of Compound (20B) to Formula (IV) is 0.001 to 0.1, e.g., 0.001 to 0.05, 0.001 to 0.01, or 0.001 to 0.005 of Compound (20B) to Formula (IV). In another embodiment, the compound having Formula (IV) is Compound (9). In another embodiment, the compound having Formula (IV) is Compound (10).

In another embodiment, the present disclosure provides a composition comprising a compound having Formula (IV), or a salt or solvate thereof, and Compound (21), or a salt or solvate thereof, wherein the weight ratio of Compound (21) to Formula (IV) is 0.001 to 0.1, e.g., 0.001 to 0.05, 0.001 to 0.01, or 0.001 to 0.005 of Compound (21) to Formula (IV). In another embodiment, the compound having Formula (IV) is Compound (9). In another embodiment, the compound having Formula (IV) is Compound (10).

In another embodiment, the present disclosure provides a composition comprising Compound (10), or a salt or solvate thereof, and Compound (2), or a salt or solvate thereof.

In another embodiment, the present disclosure provides a composition comprising Compound (10), or a salt or solvate thereof, and Compound (2), or a salt or solvate thereof, wherein the weight ratio of Compound (2) to Compound (10) is 0.0001 to 0.1, e.g., 0.001 to 0.05, 0.001 to 0.01, or 0.001 to 0.005 of Compound (2) to Compound (10).

In another embodiment, the present disclosure provides a composition comprising Compound (10), or a salt or solvate thereof, and Compound (21), or a salt or solvate thereof.

In another embodiment, the present disclosure provides a composition comprising Compound (10), or a salt or solvate thereof, and Compound (21), or a salt or solvate thereof, wherein the weight ratio of Compound (21) to Compound (10) is 0.0001 to 0.1, e.g., 0.001 to 0.05, 0.001 to 0.01, or 0.001 to 0.005 of Compound (21) to Compound (10).

In another embodiment, the present disclosure provides a composition comprising Compound (10), or a salt or solvate thereof, Compound (2), or a salt or solvate thereof, and Compound (21), or a salt or solvate thereof.

In another embodiment, the present disclosure provides a composition comprising Compound (10), or a salt or solvate thereof, Compound (2), or a salt or solvate thereof, and Compound (21), or a salt or solvate thereof, wherein the weight ratio of Compound (2) to Compound (10) is 0.0001 to 0.1, e.g., 0.001 to 0.05, 0.001 to 0.01, or 0.001 to 0.005 of Compound (2) to Compound (10), and the weight ratio of Compound (21) to Compound (10) is 0.0001 to 0.1, e.g., 0.001 to 0.05, 0.001 to 0.01, or 0.001 to 0.005 of Compound (21) to Compound (10).

In another embodiment, the present disclosure provides a composition comprising a compound having Formula (IV), or a salt or solvate thereof, and Compound (22), or a salt or solvate thereof, wherein the weight ratio of Compound (22) to Formula (IV) is 0.001 to 0.1, e.g., 0.001 to 0.05, 0.001 to 0.01, or 0.001 to 0.005 of Compound (22) to Formula (IV). In another embodiment, the compound having Formula (IV) is Compound (9). In another embodiment, the compound having Formula (IV) is Compound (10).

In another embodiment, the present disclosure provides a composition comprising Compound (10), or a salt or solvate thereof, and Compound (22), or a salt or solvate thereof.

In another embodiment, the present disclosure provides a composition comprising Compound (10), or a salt or solvate thereof, and Compound (22), or a salt or solvate thereof, wherein the weight ratio of Compound (22) to Compound (10) is 0.0001 to 0.1, e.g., 0.001 to 0.05, 0.001 to 0.01, or 0.001 to 0.005 of Compound (22) to Compound (10).

In another embodiment, the present disclosure provides a composition comprising Compound (10), or a salt or solvate thereof, Compound (2), or a salt or solvate thereof, and Compound (22), or a salt or solvate thereof.

In another embodiment, the present disclosure provides a composition comprising Compound (10), or a salt or solvate thereof, Compound (2), or a salt or solvate thereof, and Compound (22), or a salt or solvate thereof, wherein the weight ratio of Compound (2) to Compound (10) is 0.0001 to 0.1, e.g., 0.001 to 0.05, 0.001 to 0.01, or 0.001 to 0.005 of Compound (2) to Compound (10), and the weight ratio of Compound (22) to Compound (10) is 0.0001 to 0.1, e.g., 0.001 to 0.05, 0.001 to 0.01, or 0.001 to 0.005 of Compound (22) to Compound (10).

In another embodiment, the present disclosure provides a composition comprising a compound having Formula (IV), or a salt or solvate thereof, and Compound (23), or a salt or solvate thereof, wherein the weight ratio of Compound (23) to Formula (IV) is 0.0001 to 0.1, e.g., 0.001 to 0.05, 0.001 to 0.01, or 0.001 to 0.005 of Compound (23) to Formula (IV). In another embodiment, the compound having Formula (IV) is Compound (9). In another embodiment, the compound having Formula (IV) is Compound (10).

In another embodiment, the present disclosure provides a composition comprising Compound (10), or a salt or solvate thereof, and Compound (23), or a salt or solvate thereof.

In another embodiment, the present disclosure provides a composition comprising Compound (10), or a salt or solvate thereof, and Compound (23), or a salt or solvate thereof, wherein the weight ratio of Compound (23) to Compound (10) is 0.0001 to 0.1, e.g., 0.001 to 0.05, 0.001 to 0.01, or 0.001 to 0.005 of Compound (23) to Compound (10).

In another embodiment, the present disclosure provides a composition comprising a compound having Formula (IV), or a salt or solvate thereof, and Compound (24), or a salt or solvate thereof, wherein the weight ratio of Compound (24) to Formula (IV) is 0.0001 to 0.1, e.g., 0.001 to 0.05, 0.001 to 0.01, or 0.001 to 0.005 of Compound (24) to Formula (IV). In another embodiment, the compound having Formula (IV) is Compound (9). In another embodiment, the compound having Formula (IV) is Compound (10).

In another embodiment, the present disclosure provides a composition comprising Compound (10), or a salt or solvate thereof, and Compound (24), or a salt or solvate thereof.

In another embodiment, the present disclosure provides a composition comprising Compound (10), or a salt or solvate thereof, and Compound (24), or a salt or solvate thereof, wherein the weight ratio of Compound (24) to Compound (10) is 0.0001 to 0.1, e.g., 0.001 to 0.05, 0.001 to 0.01, or 0.001 to 0.005 of Compound (24) to Compound (10).

In another embodiment, the present disclosure provides a composition comprising a compound of Table 1.

In another embodiment, the present disclosure provides a composition comprising Compound (10) and at least one other compound of Table 1.

In another embodiment, the present disclosure provides a composition comprising about 80% to about 99% of Compound (10), e.g., about 85% to about 99%, about 90% to about 99%, or about 95% to about 99% of Compound (10), and 0.0001% to 5%, e.g., 0.001% to 3%, 0.001 to 1%, 0.01% to 1%, or 0.1% to 1%, of at least one other compound of Table 1.

In another embodiment, the present disclosure provides a composition comprising about 80% to about 99% of Compound (10), e.g., about 85% to about 99%, about 90% to about 99%, or about 95% to about 99% of Compound (10), and 0.0001% to 5% of Compound (2), e.g., 0.001% to 3%, 0.001 to 1%, 0.01% to 1%, or 0.1% to 1% of Compound (2).

In another embodiment, the present disclosure provides a composition comprising about 80% to about 99% of Compound (10), e.g., about 85% to about 99%, about 90% to about 99%, or about 95% to about 99% of Compound (10), and 0.0001% to 5% of Compound (21), e.g., 0.001% to 3%, 0.001 to 1%, 0.01% to 1%, or 0.1% to 1% of Compound (21).

In another embodiment, the present disclosure provides a composition comprising about 80% to about 99% of Compound (10), e.g., about 85% to about 99%, about 90% to about 99%, or about 95% to about 99% of Compound (10), and 0.0001% to 5% of Compound (2), e.g., 0.001% to 3%, 0.001 to 1%, 0.01% to 1%, or 0.1% to 1%, of Compound (2), and 0.0001% to 5% of Compound (21), e.g., 0.001% to 3%, 0.001 to 1%, 0.01% to 1%, or 0.1% to 1% of Compound (21).

In another embodiment, the present disclosure provides a composition comprising about 80% to about 99% of Compound (10), e.g., about 85% to about 99%, about 90% to about 99%, or about 95% to about 99% of Compound (10), and 0.0001% to 5% of Compound (22), e.g., 0.001% to 3%, 0.001 to 1%, 0.01% to 1%, or 0.1% to 1% of Compound (22).

In another embodiment, the present disclosure provides a composition comprising about 80% to about 99% of Compound (10), e.g., about 85% to about 99%, about 90% to about 99%, or about 95% to about 99% of Compound (10), and 0.0001% to 5% of Compound (23), e.g., 0.001% to 3%, 0.001 to 1%, 0.01% to 1%, or 0.1% to 1% of Compound (23).

In another embodiment, the present disclosure provides a composition comprising about 80% to about 99% of Compound (10), e.g., about 85% to about 99%, about 90% to about 99%, or about 95% to about 99% of Compound (10), and 0.0001% to 5% of Compound (24), e.g., 0.001% to 3%, 0.001 to 1%, 0.01% to 1%, or 0.1% to 1% of Compound (24).

The present disclosure provides the following particular composition embodiments with respect to compositions comprising Compound (10).

Composition Embodiment 1. A composition comprising:
(i) Compound (10), or a salt or solvate thereof; and
(ii) one or more molecules, or a salt or solvate thereof,
wherein the one or more molecules are selected from the group consisting of Compounds (1)-(9), (11A), (11B), (12)-(19), (20A), (20B), (21)-(26), (27A), (27B), and (28)-(50) of Table 1, or a combination thereof.

Composition Embodiment 2. The composition of Composition Embodiment 1, wherein the composition comprises Compound (1) as a molecule.

Composition Embodiment 3. The composition of Composition Embodiments 1 or 2, wherein the composition comprises Compound (2) as a molecule.

Composition Embodiment 4. The composition of any one of Composition Embodiments 1-3, wherein the composition comprises Compound (3) as a molecule.

Composition Embodiment 5. The composition of any one of Composition Embodiments 1-4, wherein the composition comprises Compound (4) as a molecule.

Composition Embodiment 6. The composition of any one of Composition Embodiments 1-5, wherein the composition comprises Compound (5) as a molecule.

Composition Embodiment 7. The composition of any one of Composition Embodiments 1-6, wherein the composition comprises Compound (6) as a molecule.

Composition Embodiment 8. The composition of any one of Composition Embodiments 1-7, wherein the composition comprises Compound (7) as a molecule.

Composition Embodiment 9. The composition of any one of Composition Embodiments 1-8, wherein the composition comprises Compound (8) as a molecule.

Composition Embodiment 10. The composition of any one of Composition Embodiments 1-9, wherein the composition comprises Compound (9) as a molecule.

Composition Embodiment 11. The composition of any one of Composition Embodiments 1-10, wherein the composition comprises Compound (11A) as a molecule.

Composition Embodiment 12. The composition of any one of Composition Embodiments 1-11, wherein the composition comprises Compound (11B) as a molecule.

Composition Embodiment 13. The composition of any one of Composition Embodiments 1-12, wherein the composition comprises Compound (12) as a molecule.

Composition Embodiment 14. The composition of any one of Composition Embodiments 1-12, wherein the composition comprises Compound (13) as a molecule.

Composition Embodiment 15. The composition of any one of Composition Embodiments 1-14, wherein the composition comprises Compound (14) as a molecule.

Composition Embodiment 16. The composition of any one of Composition Embodiments 1-15, wherein the composition comprises Compound (15) as a molecule.

Composition Embodiment 17. The composition of any one of Composition Embodiments 1-16, wherein the composition comprises Compound (16) as a molecule.

Composition Embodiment 18. The composition of any one of Composition Embodiments 1-17, wherein the composition comprises Compound (17) as a molecule.

Composition Embodiment 19. The composition of any one of Composition Embodiments 1-18, wherein the composition comprises Compound (18) as a molecule.

Composition Embodiment 20. The composition of any one of Composition Embodiments 1-19, wherein the composition comprises Compound (19) as a molecule.

Composition Embodiment 21. The composition of any one of Composition Embodiments 1-20, wherein the composition comprises Compound (20A) as a molecule.

Composition Embodiment 22. The composition of any one of Composition Embodiments 1-21, wherein the composition comprises Compound (20B) as a molecule.

Composition Embodiment 23. The composition of any one of Composition Embodiments 1-22, wherein the composition comprises Compound (21) as a molecule.

Composition Embodiment 24. The composition of any one of Composition Embodiments 1-23, wherein the composition comprises Compound (22) as a molecule.

Composition Embodiment 25. The composition of any one of Composition Embodiments 1-24, wherein the composition comprises Compound (23) as a molecule.

Composition Embodiment 26. The composition of any one of Composition Embodiments 1-25, wherein the composition comprises Compound (24) as a molecule.

Composition Embodiment 27. The composition of any one of Composition Embodiments 1-26, wherein the composition comprises Compound (25) as a molecule.

Composition Embodiment 28. The composition of any one of Composition Embodiments 1-27, wherein the composition comprises Compound (26) as a molecule.

Composition Embodiment 29. The composition of any one of Composition Embodiments 1-28, wherein the composition comprises Compound (27A) as a molecule.

Composition Embodiment 30. The composition of any one of Composition Embodiments 1-29, wherein the composition comprises Compound (27B) as a molecule.

Composition Embodiment 31. The composition of any one of Composition Embodiments 1-30, wherein the composition comprises Compound (28) as a molecule.

Composition Embodiment 32. The composition of any one of Composition Embodiments 1-31, wherein the composition comprises Compound (29) as a molecule.

Composition Embodiment 33. The composition of any one of Composition Embodiments 1-32, wherein the composition comprises Compound (30) as a molecule.

Composition Embodiment 34. The composition of any one of Composition Embodiments 1-33, wherein the composition comprises Compound (31) as a molecule.

Composition Embodiment 35. The composition of any one of Composition Embodiments 1-34, wherein the composition comprises Compound (32) as a molecule.

Composition Embodiment 36. The composition of any one of Composition Embodiments 1-35, wherein the composition comprises Compound (33) as a molecule.

Composition Embodiment 37. The composition of any one of Composition Embodiments 1-36, wherein the composition comprises Compound (34) as a molecule.

Composition Embodiment 38. The composition of any one of Composition Embodiments 1-37, wherein the composition comprises Compound (35) as a molecule.

Composition Embodiment 39. The composition of any one of Composition Embodiments 1-38, wherein the composition comprises Compound (36) as a molecule.

Composition Embodiment 40. The composition of any one of Composition Embodiments 1-39, wherein the composition comprises Compound (37) as a molecule.

Composition Embodiment 41. The composition of any one of Composition Embodiments 1-40, wherein the composition comprises Compound (38) as a molecule.

Composition Embodiment 42. The composition of any one of Composition Embodiments 1-41, wherein the composition comprises Compound (39) as a molecule.

Composition Embodiment 43. The composition of any one of Composition Embodiments 1-2, wherein the composition comprises Compound (40) as a molecule.

Composition Embodiment 44. The composition of any one of Composition Embodiments 1-43, wherein the composition comprises Compound (41) as a molecule.

Composition Embodiment 45. The composition of any one of Composition Embodiments 1-44, wherein the composition comprises Compound (42) as a molecule.

Composition Embodiment 46. The composition of any one of Composition Embodiments 1-45, wherein the composition comprises Compound (43) as a molecule.

Composition Embodiment 47. The composition of any one of Composition Embodiments 1-46, wherein the composition comprises Compound (44) as a molecule.

Composition Embodiment 48. The composition of any one of Composition Embodiments 1-47, wherein the composition comprises Compound (45) as a molecule.

Composition Embodiment 49. The composition of any one of Composition Embodiments 1-48, wherein the composition comprises Compound (46) as a molecule.

Composition Embodiment 50. The composition of any one of Composition Embodiments 1-49, wherein the composition comprises Compound (47) as a molecule.

Composition Embodiment 51. The composition of any one of Composition Embodiments 1-50, wherein the composition comprises Compound (48) as a molecule.

Composition Embodiment 52. The composition of any one of Composition Embodiments 1-51, wherein the composition comprises Compound (49) as a molecule.

Composition Embodiment 53. The composition of any one of Composition Embodiments 1-52, wherein the composition comprises Compound (50) as a molecule

Composition Embodiment 54. The composition of Composition Embodiment 1, wherein the composition comprises Compound (2) and Compound (21) as molecules.

Composition Embodiment 55. The composition of Composition Embodiment 1, wherein the composition comprises Compound (2) and Compound (25) as molecules.

Composition Embodiment 56. The composition of Composition Embodiment 1, wherein the composition comprises Compound (2) and Compound (37) as molecules.

Composition Embodiment 57. The composition of Composition Embodiment 1, wherein the composition comprises Compound (21) and Compound (25) as molecules.

Composition Embodiment 58. The composition of Composition Embodiment 1, wherein the composition comprises Compound (21) and Compound (37) as molecules.

Composition Embodiment 59. The composition of Composition Embodiment 1, wherein the composition comprises Compound (25) and Compound (37) as molecules.

Composition Embodiment 60. The composition of Composition Embodiment 1, wherein the composition comprises Compound (2), Compound (21), and Compound (25) as molecules.

Composition Embodiment 61. The composition of Composition Embodiment 1, wherein the composition comprises Compound (2), Compound (21), and Compound (37) as molecules.

Composition Embodiment 62. The composition of Composition Embodiment 1, wherein the composition comprises Compound (2), Compound (25), and Compound (37) as molecules.

Composition Embodiment 63. The composition of Composition Embodiment 1, wherein the composition comprises Compound (21), Compound (25), and Compound (37) as molecules.

Composition Embodiment 64. The composition of Composition Embodiment 1, wherein the composition comprises Compound (2), Compound (21), Compound (25), and Compound (37) as molecules.

Composition Embodiment 65. The composition of any one of Composition Embodiments 5-66 comprising about 25% to about 99% of Compound (10) as measured by HPLC and/or GC analysis.

Composition Embodiment 66. The composition of Composition Embodiment 65 comprising about 50% to about 99% of Compound (10) as measured by HPLC and/or GC analysis.

Composition Embodiment 67. The composition of Composition Embodiment 67 comprising about 89% to about 99% of Compound (10) as measured by HPLC and/or GC analysis.

Composition Embodiment 68. The composition of any one of Composition Embodiments 5-66 comprising about 10%, about 15%, about 20%, about 25%, about 30%, about 35%, about 40%, about 45%, about 50%, about 55%, about 60%, about 65%, about 70%, about 80%, about 85%, about 90%, about 95%, about 96%, about 97%, about 98%, or about 99% of Compound (10) as measured by HPLC and/or GC analysis.

Composition Embodiment 69. The composition of any one of Composition Embodiments 5-68 comprising about 0.000001% to about 10% of any one or more molecules as measured by HPLC and/or GC analysis.

Composition Embodiment 70. The composition of Composition Embodiment 69 comprising about 0.000001% to about 5% of any one or more molecules as measured by HPLC and/or GC analysis.

Composition Embodiment 71. The composition of Composition Embodiment 69 comprising about 0.0001% to about 10% of any one or more molecules as measured by HPLC and/or GC analysis.

Composition Embodiment 72. The composition of Composition Embodiment 71 comprising about 0.0001% to about 5% of any one or more molecules as measured by HPLC and/or GC analysis.

Composition Embodiment Composition Embodiment 72. The composition of Composition Embodiment 72 comprising about 0.001% to about 3% of any one or more molecules as measured by HPLC and/or GC analysis.

Composition Embodiment 73. The composition of any one of Composition Embodiments 5-68, wherein the weight ratio of any one or more molecules to Compound (10) is 0.000001 to 0.1.

Composition Embodiment 74. A composition comprising Compound (10); and
(i) about 0.000001% to about 5% of Compound (2) based on HPLC analysis;
(ii) about 0.000001% to about 5% of Compound (21) based on HPLC analysis;
(iii) about 0.000001% to about 5% of Compound (25) based on HPLC analysis; and/or
(iv) about 0.000001% to about 5% of Compound (37) based on HPLC analysis.

Composition Embodiment 75. The composition of Composition Embodiment 74 comprising about 0.000001% to about 2% of Compound (2) based on HPLC analysis.

Composition Embodiment 76. The composition of Composition Embodiment 75 comprising about 0.000001% to about 1% of Compound (2) based on HPLC analysis.

Composition Embodiment 77. The composition of Composition Embodiment 76 comprising about 0.000001% to about 0.5% of Compound (2) based on HPLC analysis.

Composition Embodiment 78. The composition of Composition Embodiment 77 comprising about 0.000001% to about 0.25% of Compound (2) based on HPLC analysis.

Composition Embodiment 79. The composition of Composition Embodiment 78 comprising about 0.000001% to about 0.1% of Compound (2) based on HPLC analysis.

Composition Embodiment 80. The composition of Composition Embodiment 76 comprising about 0.001% to about 1% of Compound (2) based on HPLC analysis.

Composition Embodiment 81. The composition of Composition Embodiment 80 comprising about 0.01% to about 1% of Compound (2) based on HPLC analysis.

Composition Embodiment 82. The composition of Composition Embodiment 81 comprising about 0.1% to about 1% of Compound (2) based on HPLC analysis.

Composition Embodiment 83. The composition of any one of Composition Embodiments 74-82 comprising about 0.000001% to about 2% of Compound (21) based on HPLC analysis.

Composition Embodiment 84. The composition of Composition Embodiment 83 comprising about 0.000001% to about 1% of Compound (21) based on HPLC analysis.

Composition Embodiment 85. The composition of Composition Embodiment 84 comprising about 0.000001% to about 0.5% of Compound (21) based on HPLC analysis.

Composition Embodiment 86. The composition of Composition Embodiment 85 comprising about 0.000001% to about 0.25% of Compound (21) based on HPLC analysis.

Composition Embodiment 87. The composition of Composition Embodiment 86 comprising about 0.000001% to about 0.1% of Compound (21) based on HPLC analysis.

Composition Embodiment 88. The composition of Composition Embodiment 84 comprising about 0.001% to about 1% of Compound (21) based on HPLC analysis.

Composition Embodiment 89. The composition of Composition Embodiment 88 comprising about 0.01% to about 1% of Compound (21) based on HPLC analysis.

Composition Embodiment 90. The composition of Composition Embodiment 89 comprising about 0.1% to about 1% of Compound (21) based on HPLC analysis.

Composition Embodiment 91. The composition of any one of Composition Embodiments 74-90 comprising about 0.000001% to about 2% of Compound (25) based on HPLC analysis.

Composition Embodiment 92. The composition of Composition Embodiment 91 comprising about 0.000001% to about 1% of Compound (25) based on HPLC analysis.

Composition Embodiment 93. The composition of Composition Embodiment 92 comprising about 0.000001% to about 0.5% of Compound (25) based on HPLC analysis.

Composition Embodiment 94. The composition of Composition Embodiment 93 comprising about 0.000001% to about 0.25% of Compound (25) based on HPLC analysis.

Composition Embodiment 95. The composition of Composition Embodiment 94 comprising about 0.000001% to about 0.1% of Compound (25) based on HPLC analysis.

Composition Embodiment 96. The composition of Composition Embodiment 92 comprising about 0.001% to about 1% of Compound (25) based on HPLC analysis.

Composition Embodiment 97. The composition of Composition Embodiment 96 comprising about 0.01% to about 1% of Compound (25) based on HPLC analysis.

Composition Embodiment 98. The composition of Composition Embodiment 97 comprising about 0.1% to about 1% of Compound (25) based on HPLC analysis.

Composition Embodiment 99. The composition of any one of Composition Embodiments 74-98 comprising about 0.000001% to about 2% of Compound (37) based on HPLC analysis.

Composition Embodiment 100. The composition of Composition Embodiment 99 comprising about 0.000001% to about 1% of Compound (37) based on HPLC analysis.

Composition Embodiment 101. The composition of Composition Embodiment 100 comprising about 0.000001% to about 0.5% of Compound (37) based on HPLC analysis.

Composition Embodiment 102. The composition of Composition Embodiment 101 comprising about 0.000001% to about 0.25% of Compound (37) based on HPLC analysis.

Composition Embodiment 103. The composition of Composition Embodiment 102 comprising about 0.000001% to about 0.1% of Compound (37) based on HPLC analysis.

Composition Embodiment 104. The composition of Composition Embodiment 100 comprising about 0.001% to about 1% of Compound (37) based on HPLC analysis.

Composition Embodiment 105. The composition of Composition Embodiment 104 comprising about 0.01% to about 1% of Compound (37) based on HPLC analysis.

Composition Embodiment 106. The composition of Composition Embodiment 105 comprising about 0.1% to about 1% of Compound (37) based on HPLC analysis.

Composition Embodiment 107. The composition of any one of Composition Embodiments 74-106 comprising about 50% to about 99.9% of Compound (10) based on HPLC analysis.

Composition Embodiment 108. The composition of Composition Embodiment 107 comprising about 75% to about 99.9% of Compound (10) based on HPLC analysis.

Composition Embodiment 109. The composition of Composition Embodiment 108 comprising about 80% to about 99.9% of Compound (10) based on HPLC analysis.

Composition Embodiment 110. The composition of Composition Embodiment 109 comprising about 85% to about 99.9% of Compound (10) based on HPLC analysis.

Composition Embodiment 111. The composition of Composition Embodiment 110 comprising about 90% to about 99.9% of Compound (10) based on HPLC analysis.

Composition Embodiment 112. The composition of Composition Embodiment 111 comprising about 95% to about 99.9% of Compound (10) based on HPLC analysis.

The compositions of this Section II are collectively referred to as “Compositions of the Disclosure” and individually as a “Composition of the Disclosure.” III. Tire Compositions of the Disclosure

In another embodiment, the present disclosure provides compositions (collectively referred to as “Tire Compositions of the Disclosure” or individually as a “Tire Composition of the Disclosure”) comprising:
(i) a Compound or Composition of the Disclosure; and
(ii) one or more elastomers; or
(iii) one or more fillers; or
(iv) one or more rubber chemicals; or
(v) one or more plasticizers; or
(vi) one or more second antidegradants; or
(vii) a combination of one or more elastomers, one or more fillers, one or more rubber chemicals, one or more plasticizers, and/or one or more second antidegradants.

In some embodiments, a Tire Composition of the Disclosure comprises from about 0.1 wt/wt % to about 99 wt/wt %, e.g., about 15 wt/wt % to about 85 wt/wt % of a Compound or Composition of the Disclosure. In some embodiments, the composition comprises from from about 0.1 wt/wt % to about 1 wt/wt %, about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %. from about 25 wt/wt % to about 45 wt/wt %. from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt % from about 35 wt/wt % to about 65 wt/wt % from about 35 wt/wt % to about 75 wt/wt % from about 35 wt/wt % to about 85 wt/wt % from about 35 wt/wt % to about 95 wt/wt % from about 45 wt/wt % to about 55 wt/wt % from about 45 wt/wt % to about 65 wt/wt % from about 45 wt/wt % to about 75 wt/wt % from about 45 wt/wt % to about 85 wt/wt % from about 45 wt/wt % to about 95 wt/wt % from about 55 wt/wt % to about 65 wt/wt % from about 55 wt/wt % to about 75 wt/wt % from about 55 wt/wt % to about 85 wt/wt % from about 55 wt/wt % to about 95 wt/wt % from about 65 wt/wt % to about 75 wt/wt % from about 65 wt/wt % to about 85 wt/wt % from about 65 wt/wt % to about 95 wt/wt % from about 75 wt/wt % to about 85 wt/wt % from about 75 wt/wt % to about 95 wt/wt % or from about 85 wt/wt % to about 95 wt/wt % of a Compound or Composition of the Disclosure.

In some embodiments, a Tire Composition of the Disclosure comprises about 50 wt/wt % of a Compound or Composition of the Disclosure. In some embodiments, the composition comprises about 1 wt/wt %, about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of a Compound or Composition of the Disclosure.

In another embodiment, a Tire Composition of the Disclosure comprises a Compound or Composition of the Disclosure and one or more elastomers.

The term “elastomer” as used herein is a polymer with viscoelasticity (i.e., having both viscosity and elasticity) that typically has low intermolecular forces, low Young's modulus, and high failure strain. Elastomers can typically be cross-linked by heating in the presence of one or more cross-linking agents, a process called curing or vulcanization. Rubber is one type of elastomer. Non-limiting types of rubber include natural rubber (NR), synthetic rubber, and blends thereof The term “natural rubber” as used herein refers to a naturally occurring elastomer that can be obtained from Hevea rubber trees. Non-limiting types of synthetic rubbers include unsaturated rubbers, saturated rubbers, rubbers with fluoro and fluoralkyl or fluoralkoxy substituent groups on the polymer chain (FKM), silicone rubbers (Q), and blends thereof Non-limiting examples of unsaturated rubbers include polyisoprene rubber (IR), butyl rubber (IIR), polybutadiene rubber (BR), styrene-isoprene-butadiene rubber (SIBR), styrene butadiene rubber (SBR), nitrile butadiene rubber (NBR), chloroprene rubber (CR), ethylene propylene diene rubber (EPDM), and blends thereof. These unsaturated rubbers undergo cyclization and crosslinking reactions that lead to hardening of the aged part. As oxidation occurs, these vulcanizates harden and eventually become brittle products. Partial oxidation of vulcanizates leads to losses in performance when used in applications such as vehicle tire sidewalls. Saturated rubbers are rubbers that do not contain C═C unsaturation and include, but are not limited to, acrylic rubber (ACM), chlorinated polyethylene (CM), chlorosulfonated polyethylene (CSM), polychloromethyloxiran (CO), ethylene-ethyl acrylate copolymer (EAM), epichlorohydrin rubber (ECO), ethylene propylene rubber (EPM), ethylenevinylacetate copolymer (EVM), rubbers with fluoro and fluoralkyl or fluoralkoxy substituent groups on the polymer chain (FKM), silicone rubber (Q), and blends thereof.

In some embodiments, the natural rubber comprises rubber derived from an alternative rubber plant. The term “natural rubber comprises rubber derived from an alternative rubber plant” as used herein refers to a naturally occurring elastomer that can be obtained from “non-Hevea” sources. In some embodiments, the alternative rubber plant is Parthenium argentatum (guayule) or Taraxacum kok-saghyz (Russian dandelion).

In some embodiments, the one or more elastomers further comprises recycled rubber. The term “recycled rubber” as used herein refers to an elastomer that has been reclaimed from scrap materials such as used tires.

In some embodiments, a Tire Composition of the Disclosure comprises from about 15 wt/wt % to about 99 wt/wt % of one or more elastomers. In some embodiments, a Tire Composition of the Disclosure comprises from about 15 wt/wt % to about 85 wt/wt % of one or more elastomers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt % from about 1 wt/wt % to about 95 wt/wt %, from about 5 wt/wt % to about 15 wt/wt % from about 5 wt/wt % to about 25 wt/wt %, from about 5 wt/wt % to about 35 wt/wt %, from about 5 wt/wt % to about 45 wt/wt % from about 5 wt/wt % to about 55 wt/wt % from about 5 wt/wt % to about 65 wt/wt %. from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt % from about 15 wt/wt % to about 45 wt/wt % from about 15 wt/wt % to about 55 wt/wt % from about 15 wt/wt % to about 65 wt/wt % from about 15 wt/wt % to about 75 wt/wt %, from about 15 wt/wt % to about 95 wt/wt % from about 25 wt/wt % to about 35 wt/wt % from about 25 wt/wt % to about 45 wt/wt % from about 25 wt/wt % to about 55 wt/wt % from about 25 wt/wt % to about 65 wt/wt % from about 25 wt/wt % to about 75 wt/wt %, from about 25 wt/wt % to about 85 wt/wt % from about 25 wt/wt % to about 95 wt/wt % from about 35 wt/wt % to about 45 wt/wt % from about 35 wt/wt % to about 55 wt/wt % from about 35 wt/wt % to about 65 wt/wt % from about 35 wt/wt % to about 75 wt/wt %, from about 35 wt/wt % to about 85 wt/wt % from about 35 wt/wt % to about 95 wt/wt % from about 45 wt/wt % to about 55 wt/wt % from about 45 wt/wt % to about 65 wt/wt % from about 45 wt/wt % to about 75 wt/wt % from about 45 wt/wt % to about 85 wt/wt %, from about 45 wt/wt % to about 95 wt/wt % from about 55 wt/wt % to about 65 wt/wt % from about 55 wt/wt % to about 75 wt/wt % from about 55 wt/wt % to about 85 wt/wt % from about 55 wt/wt % to about 95 wt/wt % from about 65 wt/wt % to about 75 wt/wt %, from about 65 wt/wt % to about 85 wt/wt % from about 65 wt/wt % to about 95 wt/wt % from about 75 wt/wt % to about 85 wt/wt % from about 75 wt/wt % to about 95 wt/wt %, or from about 85 wt/wt % to about 95 wt/wt % of one or more elastomers.

In some embodiments, a Tire Composition of the Disclosure comprises about 50 wt/wt % of one or more elastomers. In some embodiments, the composition comprises about 1 wt/wt %, about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more elastomers.

The term “phr” as used herein refers to parts per hundred parts of rubber by weight. The parts by weight of individual components are based on 100 parts by weight of the total mass of the one or more elastomers present in the composition.

In some embodiments, a Tire Composition of the Disclosure comprises from about 1 phr to about 5 phr of a Compound or Composition of the Disclosure. In some embodiments, the composition comprises from about 0.01 phr to about 0.1 phr, from about 0.01 phr to about 0.5 phr, from about 0.01 phr to about 1 phr, from about 0.01 phr to about 2 phr, from about 0.01 phr to about 3 phr, from about 0.01 phr to about 4 phr, from about 0.01 phr to about 5 phr, from about 0.01 phr to about 7.5 phr, from about 0.01 phr to about 10 phr, from about 0.01 phr to about 20 phr, from about 0.1 phr to about 0.5 phr, from about 0.1 phr to about 1 phr, from about 0.1 phr to about 2 phr, from about 0.1 phr to about 3 phr, from about 0.1 phr to about 4 phr, from about 0.1 phr to about 5 phr, from about 0.1 phr to about 7.5 phr, from about 0.1 phr to about 10 phr, from about 0.1 phr to about 20 phr, from about 1 phr to about 2 phr, from about 1 phr to about 3 phr, from about 1 phr to about 4 phr, from about 1 phr to about 7.5 phr, from about 1 phr to about 10 phr, from about 1 phr to about 20 phr, from about 2 phr to about 3 phr, from about 2 phr to about 4 phr, from about 2 phr to about 5 phr, from about 2 phr to about 7.5 phr, from about 2 phr to about 10 phr, from about 2 phr to about 20 phr, from about 3 phr to about 4 phr, from about 3 phr to about 5 phr, from about 3 phr to about 7.5 phr, from about 3 phr to about 10 phr, from about 3 phr to about 20 phr, from about 4 phr to about 5 phr, from about 4 phr to about 7.5 phr, from about 4 phr to about 10 phr, from about 4 phr to about 20 phr, from about 5 phr to about 7.5 phr, from about 5 phr to about 10 phr, from about 5 phr to about 20 phr, from about 7.5 phr to about 10 phr, from about 7.5 phr to about 20 phr, or from about 10 phr to about 20 phr of a Compound or Composition of the Disclosure.

In some embodiments, a Tire Composition of the Disclosure comprises about 3 phr of a Compound or Composition of the Disclosure. In some embodiments, the composition comprises about 0.01 phr, about 0.1 phr, about 0.5 phr, about 1 phr, about 2 phr, about 3 phr, about 4 phr, about 5 phr, about 7.5 phr, about 10 phr, or about 20 phr of a Compound or Composition of the Disclosure.

In some embodiments, a Tire Composition of the Disclosure comprises a Compound or Composition of the Disclosure and one or more fillers.

The term “filler” as used herein is a substance that reinforces an elastomeric composition or gives an elastomeric composition other properties, including but not limited to expanding the volume of the composition. Non-limiting examples of fillers include carbon black, silica, kaolin, calcium silicate, talc, carbon nanotubes (CNT), carbon fibers (HCF), graphite, graphenes, aluminosilicates, starch, and fibers, and combinations thereof.

In some embodiments, the filler is derived from natural sources. For example, silica may be derived from rice husks.

In some embodiments, a Tire Composition of the Disclosure comprises from about 15 wt/wt % to about 85 wt/wt % of one or more fillers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt % from about 5 wt/wt % to about 15 wt/wt %, from about 5 wt/wt % to about 25 wt/wt % from about 5 wt/wt % to about 35 wt/wt %, from about 5 wt/wt % to about 45 wt/wt % from about 5 wt/wt % to about 55 wt/wt %, from about 5 wt/wt % to about 65 wt/wt % from about 5 wt/wt % to about 75 wt/wt %, from about 5 wt/wt % to about 85 wt/wt % from about 5 wt/wt % to about 95 wt/wt % from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt % from about 75 wt/wt % to about 95 wt/wt % or from about 85 wt/wt % to about 95 wt/wt % of one or more fillers.

In some embodiments, a Tire Composition of the Disclosure comprises about 50 wt/wt % of one or more fillers. In some embodiments, the composition comprises about 1 wt/wt %. about 5 wt/wt %. about 10 wt/wt %. about 15 wt/wt %. about 20 wt/wt %. about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more fillers.

In some embodiments, a Tire Composition of the Disclosure comprises from about 30 phr to about 500 phr of one or more fillers. In some embodiments, the composition comprises from about 30 phr to about 50 phr, from about 30 phr to about 100 phr, from about 30 phr to about 150 phr, from about 30 phr to about 200 phr, from about 30 phr to about 250 phr, from about 30 phr to about 300 phr, from about 30 phr to about 350 phr, from about 30 phr to about 400 phr, from about 30 phr to about 450 phr, from about 30 phr to about 500 phr, from about 50 phr to about 100 phr, from about 50 phr to about 150 phr, from about 50 phr to about 200 phr, from about 50 phr to about 250 phr, from about 50 phr to about 300 phr, from about 50 phr to about 350 phr, from about 50 phr to about 400 phr, from about 50 phr to about 450 phr, from about 50 phr to about 500 phr, from about 100 phr to about 150 phr, from about 100 phr to about 200 phr, from about 100 phr to about 250 phr, from about 100 phr to about 300 phr, from about 100 phr to about 350 phr, from about 100 phr to about 400 phr, from about 100 phr to about 450 phr, from about 100 phr to about 500 phr, from about 150 phr to about 200 phr, from about 150 phr to about 250 phr, from about 150 phr to about 300 phr, from about 150 phr to about 350 phr, from about 150 phr to about 400 phr, from about 150 phr to about 450 phr, from about 150 phr to about 500 phr, from about 200 phr to about 250 phr, from about 200 phr to about 300 phr, from about 200 phr to about 350 phr, from about 200 phr to about 400 phr, from about 200 phr to about 450 phr, from about 200 phr to about 500 phr, from about 250 phr to about 300 phr, from about 250 phr to about 350 phr, from about 250 phr to about 400 phr, from about 250 phr to about 450 phr, from about 250 phr to about 500 phr, from about 300 phr to about 350 phr, from about 300 phr to about 400 phr, from about 300 phr to about 450 phr, from about 300 phr to about 500 phr, from about 350 phr to about 400 phr, from about 350 phr to about 450 phr, from about 350 phr to about 500 phr, from about 400 phr to about 450 phr, from about 400 phr to about 500 phr, or from about 450 phr to about 500 phr of one or more fillers.

In some embodiments, a Tire Composition of the Disclosure comprises about 300 phr of one or more fillers. In some embodiments, the composition comprises about 30 phr, about 50 phr, about 100 phr, about 150 phr, about 200 phr, about 250 phr, about 350 phr, about 400 phr, about 450 phr, or about 500 phr of one or more fillers.

In some embodiments, a Tire Composition of the Disclosure comprises a Compound or Composition of the Disclosure and one or more rubber chemicals. The term “rubber chemicals” as used herein refers to a compound or substance used to facilitate the vulcanization of rubber. Rubber chemicals include, but are not limited to, vulcanizing agents, accelerators, activators, and pre-vulcanization inhibitors.

The term “vulcanization” as used herein refers to a process wherein cross-links are formed between elastomers to effect changes in the material properties of elastomers. In particular, vulcanization typically increases the rigidity and durability of elastomers. Vulcanization is carried out at room temperature or at elevated temperatures, depending on the nature of the elastomer(s), filler(s), and rubber chemical(s) being used. The term “curing” is also used in the art to describe this process.

The term “vulcanizing agent” as used herein refers to any substance that enables cross-linking between elastomers. Vulcanizing agents can enable cross-linking between separate polymer chains of an elastomer by various mechanisms, including, but not limited to, by formation of covalent bonds between the vulcanizing agent and two or more separate polymer chains or by generating radical species on separate polymer chains that can combine to form covalent bonds between the two polymer chains. Non-limiting examples of vulcanizing agents include sulfur, peroxides, vulcanized vegetable oil, factices and resins. Non-limiting examples of sulfur include octasulfur (S8), cyclododecasulfur (S12), and polymeric sulfur. Non-limiting examples of peroxides include benzoyl peroxide, dicumyl peroxide (DC), 2,5-dimethyl-2,5-di-(tert-butylperoxy)-3-hexyne (2,5 Tri), 2,5-dimethyl-2,5-di(tert-butylperoxy)hexane (DDPH), di-(2-tert-butylperoxyisopropyl)benzene (VC), butyl-4,4-di-(tert-butylperoxy)valerate (VAL), and 1,1-di(tert-butylperoxy)-3,3,5-trimethylcyclohexane (TMC). Nonlimiting examples of resins include bonding resins. The term “bonding resin” as used herein refers to a chemical such as resorcinol formaldehyde resins and phenolic resins that reacts with methylene donors (such as hexamethylenetetramine (HMTA) or hexamethoxymethyl melamine (HMMM)) to promote adhesion.

The term “accelerator” as used herein refers to any substance that increases the kinetics of vulcanization. In some embodiments, accelerators enable vulcanization to be performed at lower temperatures and/or to use the vulcanization agent, e.g., sulfur, more efficiently. Non-limiting examples of accelerators include guanidines, thiazoles, sulfenamides, thiurams, dithiocarbamates, xanthates, and thiophosphates. Non-limiting examples of guanidines include diphenylguanidine (DPG). Non-limiting examples of thiazoles include 2mercaptobenzothiazole (MBT), zinc 2mercaptobenzothiazole (ZMBT), mercaptobenzothiazole disulfide (MBTS), and N-tert-butyl-2-benzothiazole sulfenimide (TBSI). Non-limiting examples of sulfenamides include N-tert-butyl-2-benzothiazylsulfenamide (TBBS), N-cyclohexylbenzothiazol-2-sulfenamide (CBS), dicyclohexyl-2-benzothiazolesulfenamide (DCBS), N-oxydiethylene benzothiazole sulfenamide (OBTS), N-oxydiethylenethiocarbamyl-N′-oxydiethylene sulfenamide (OTOS), and thiocarbamyl sulfenamide. Non-limiting examples of thiurams include dimethylcarbamothioic dithioperoxyanhydride (thiram), dipentamethylene thiuram tetrasulfide (DPIT), tetrabenzyl thiuram disulfide (TBzTD), tetraethylthiuram disulfide (TETD), tetramethylthiuram disulfide (TMTD), and tetramethylthiuram monosulfide (TMTM). Non-limiting examples of dithiocarbamates include zinc dimethyldithiocarbamate (ZDMC), zinc diethyldithiocarbamate (ZDEC), zinc dibutyldithiocarbamate (ZDBC), nickel dibutyldithiocarbamate (NDBC), sodium dibenzyldithiocarbamate (SBEC), sodium diethyldithiocarbamate (SDEC), tellurium diethyldithiocarbamate (TDEC), and zinc dibenzyldithiocarbamate (ZEBC).

The term “activator” as used herein refers to any substance that activates a vulcanizing agent and enables it to cross-link elastomers as described above. Activators may act via various mechanisms, including, but not limited to, by forming chemical complexes with accelerators or by coordinating to sulfur (when sulfur is used as a vulcanizing agent). Non-limiting examples of activators include metal oxides, acids, and metal complexex. Non-limiting examples of metal oxides include zinc oxide, magnesium oxide, and lead oxide. Non-limiting examples of acids include stearic acid and lauric acid. Non limiting examples of metal complexes include zinc ethylhexanoate.

The term “pre-vulcanization inhibitor” as used herein refers to compounds that delay the onset and/or the rate of vulcanization. These compounds are also referred to as “retarders.” Non-limiting examples of pre-vulcanization inhibitors include N-(cyclohexylthio)phthalimide (CTP), benzoic anhydride, salicylic anhydride, and phthalic anhydride.

In some embodiments, a Tire Composition of the Disclosure comprises from about 15 wt/wt % to about 85 wt/wt % of one or more rubber chemicals. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt % from about 1 wt/wt % to about 65 wt/wt %, from about 1 wt/wt % to about 75 wt/wt % from about 1 wt/wt % to about 85 wt/wt %, from about 1 wt/wt % to about 95 wt/wt % from about 5 wt/wt % to about 15 wt/wt %, from about 5 wt/wt % to about 25 wt/wt % from about 5 wt/wt % to about 35 wt/wt %, from about 5 wt/wt % to about 45 wt/wt % from about 5 wt/wt % to about 55 wt/wt %, from about 5 wt/wt % to about 65 wt/wt % from about 5 wt/wt % to about 75 wt/wt %, from about 5 wt/wt % to about 85 wt/wt % from about 5 wt/wt % to about 95 wt/wt % from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt % from about 75 wt/wt % to about 95 wt/wt % or from about 85 wt/wt % to about 95 wt/wt % of one or more rubber chemicals.

In some embodiments, a Tire Composition of the Disclosure comprises about 15 wt/wt % of one or more rubber chemicals. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %. about 55 wt/wt %. about 60 wt/wt %. about 65 wt/wt %. about 70 wt/wt %, about 75 wt/wt %, about 80 wt/wt %, about 85 wt/wt %, about 90 wt/wt %, or about 95 wt/wt % of one or more rubber chemicals.

In some embodiments, a Tire Composition of the Disclosure comprises from about 1 phr to about 20 phr of one or more rubber chemicals. In some embodiments, the composition comprises from about 0.1 phr to about 1 phr, from about 0.1 phr to about 5 phr, from about 0.1 phr to about 10 phr, from about 0.1 phr to about 15 phr, from about 0.1 phr to about 20 phr, from about 0.1 phr to about 25 phr, from about 0.1 phr to about 30 phr, from about 0.1 phr to about 35 phr, from about 0.1 phr to about 40 phr, from about 1 phr to about 5 phr, from about 1 phr to about 10 phr, from about 1 phr to about 15 phr, from about 1 phr to about 25 phr, from about 1 phr to about 30 phr, from about 1 phr to about 35 phr, from about 1 phr to about 40 phr, from about 5 phr to about 10 phr, from about 5 phr to about 15 phr, from about 5 phr to about 20 phr, from about 5 phr to about 25 phr, from about 5 phr to about 30 phr, from about 5 phr to about 35 phr, from about 5 phr to about 40 phr, from about 10 phr to about 15 phr, from about 10 phr to about 20 phr, from about 10 phr to about 25 phr, from about 10 phr to about 30 phr, from about 10 phr to about 35 phr, from about 10 phr to about 40 phr, from about 15 phr to about 20 phr, from about 15 phr to about 25 phr, from about 15 phr to about 30 phr, from about 15 phr to about 35 phr, from about 15 phr to about 40 phr, from about 20 phr to about 25 phr, from about 20 phr to about 30 phr, from about 20 phr to about 35 phr, from about 20 phr to about 40 phr, from about 25 phr to about 30 phr, from about 25 phr to about 35 phr, from about 25 phr to about 40 phr, from about 30 phr to about 35 phr, from about 30 phr to about 40 phr, or from about 35 phr to about 40 phr of one or more rubber chemicals.

In some embodiments, a Tire Composition of the Disclosure comprises about 10 phr of one or more rubber chemicals. In some embodiments, the composition comprises about 0.1 phr, about 1 phr, about 5 phr, about 15 phr, about 20 phr, about 25 phr, about 30 phr, about 35 phr, or about 40 phr of one or more rubber chemicals.

The term “plasticizer” as used herein refers to a processing aid used to reduce the viscosity, increase the plasticity, and/or extend the volume of a composition. Plasticizers facilitate the process of mixing and forming a composition comprising an elastomer before the composition is vulcanized. Non-limiting examples of plasticizers include mineral oils (paraffinic, aromatic, or naphthenic), organic esters, resins, waxes, ester plasticizers, and naturally derived oils, such as soybean oil, vegetable oil, or orange oil.

In some embodiments, a Tire Composition of the Disclosure comprises from about 15 wt/wt % to about 85 wt/wt % of one or more plasticizers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt % from about 5 wt/wt % to about 55 wt/wt % from about 5 wt/wt % to about 65 wt/wt % from about 5 wt/wt % to about 75 wt/wt %, from about 5 wt/wt % to about 85 wt/wt % from about 5 wt/wt % to about 95 wt/wt % from about 15 wt/wt % to about 25 wt/wt %, from about 15 wt/wt % to about 35 wt/wt % from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt % from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt % from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt % from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt % from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt % from about 75 wt/wt % to about 85 wt/wt % from about 75 wt/wt % to about 95 wt/wt % or from about 85 wt/wt % to about 95 wt/wt % of one or more plasticizers.

In some embodiments, a Tire Composition of the Disclosure comprises about 15 wt/wt % of optionally one or more plasticizers. In some embodiments, the composition comprises about 1 wt/wt %. about 5 wt/wt %. about 10 wt/wt %. about 20 wt/wt %. about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more plasticizers.

In some embodiments, a Tire Composition of the Disclosure comprises from about 1 phr to about 20 phr of optionally one or more plasticizers. In some embodiments, the composition comprises from about 0.1 phr to about 1 phr, from about 0.1 phr to about 5 phr, from about 0.1 phr to about 10 phr, from about 0.1 phr to about 15 phr, from about 0.1 phr to about 20 phr, from about 0.1 phr to about 25 phr, from about 0.1 phr to about 30 phr, from about 0.1 phr to about 35 phr, from about 0.1 phr to about 40 phr, from about 1 phr to about 5 phr, from about 1 phr to about 10 phr, from about 1 phr to about 15 phr, from about 1 phr to about 25 phr, from about 1 phr to about 30 phr, from about 1 phr to about 35 phr, from about 1 phr to about 40 phr, from about 5 phr to about 10 phr, from about 5 phr to about 15 phr, from about 5 phr to about 20 phr, from about 5 phr to about 25 phr, from about 5 phr to about 30 phr, from about 5 phr to about 35 phr, from about 5 phr to about 40 phr, from about 10 phr to about 15 phr, from about 10 phr to about 20 phr, from about 10 phr to about 25 phr, from about 10 phr to about 30 phr, from about 10 phr to about 35 phr, from about 10 phr to about 40 phr, from about 15 phr to about 20 phr, from about 15 phr to about 25 phr, from about 15 phr to about 30 phr, from about 15 phr to about 35 phr, from about 15 phr to about 40 phr, from about 20 phr to about 25 phr, from about 20 phr to about 30 phr, from about 20 phr to about 35 phr, from about 20 phr to about 40 phr, from about 25 phr to about 30 phr, from about 25 phr to about 35 phr, from about 25 phr to about 40 phr, from about 30 phr to about 35 phr, from about 30 phr to about 40 phr, or from about 35 phr to about 40 phr of one or more plasticizers.

In some embodiments, a Tire Composition of the Disclosure comprises about 10 phr of one or more plasticizers. In some embodiments, the composition comprises about 0.1 phr, about 1 phr, about 5 phr, about 15 phr, about 20 phr, about 25 phr, about 30 phr, about 35 phr, or about 40 phr of one or more plasticizers.

In some embodiments, a Tire Composition of the Disclosure comprises further comprises a second or even additional antidegradant(s) that is not a Compound or Composition of the Disclosure. In some embodiments, the Additional antidegradant is an antioxidant, a heat stabilizer, a light stabilizer, or a fatigue crack stabilizer. In some embodiments, the second antidegradant is an antiozonant. Non-limiting examples of antidegradants include paraphenylenediamines (PPDs), trimethyl-dihydroquinolines (TMQs), phenolics or hindered phenolics, alkylated diphenylamines (DPAs), diphenylamine-ketone condensates, and natural antidegradants, quinones, hydroquinones, amines, hindered amines, parafinic or microcrystalline waxes. Non-limiting examples of PPDs include N1-(4-methylpentan-2-yl)-N4-phenylbenzene-1,4-diamine (6PPD), N-(1,4-dimethylpentyl)-N′uiD), 1-phenyl-N4-(propan-2-yl)benzene-1,4-diamine (IPPD), N,N′-di-sec-butyl-p-phenylenediamine (44PD), N,N′-bis(1,3-dimethylbutyl)-p-phenylenediamine (66PD), N,N′-bis(1,4-dimethylpentyl)-p-phenylenediamine (77PD), and N—N′-dioctyl-p-phenylenediamine (88PD). Non-limiting examples of TMQs include 2,2,4-trimethyl-1,2-dihydroquinoline and oligomers or polymers thereof.

In some embodiments, a Tire Composition of the Disclosure comprises from about 15 wt/wt % to about 85 wt/wt % of a second antidegradant. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt % from about 5 wt/wt % to about 15 wt/wt %, from about 5 wt/wt % to about 25 wt/wt % from about 5 wt/wt % to about 35 wt/wt %, from about 5 wt/wt % to about 45 wt/wt % from about 5 wt/wt % to about 55 wt/wt %, from about 5 wt/wt % to about 65 wt/wt % from about 5 wt/wt % to about 75 wt/wt %, from about 5 wt/wt % to about 85 wt/wt % from about 5 wt/wt % to about 95 wt/wt % from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt % from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt % from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt % from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt % from about 45 wt/wt % to about 65 wt/wt %. from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt % from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt % from about 75 wt/wt % to about 95 wt/wt % or from about 85 wt/wt % to about 95 wt/wt % of a second antidegradant.

In some embodiments, a Tire Composition of the Disclosure comprises about 15 wt/wt % of a second antidegradant. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of a second antidegradant.

In some embodiments, a Tire Composition of the Disclosure comprises from about 1 to about 5 phr of a second antidegradant. In some embodiments, the composition comprises from about 0.001 phr to about 0.01 phr, from about 0.001 phr to about 0.1 phr, from about 0.001 phr to about 1 phr, from about 0.001 phr to about 5 phr, from about 0.001 phr to about 7.5 phr, from about 0.001 phr to about 10 phr, from about 0.01 phr to about 0.1 phr, from about 0.01 phr to about 1 phr, from about 0.01 phr to about 5 phr, from about 0.01 phr to about 7.5 phr, from about 0.01 phr to about 10 phr, from about 0.1 phr to about 1 phr, from about 0.1 phr to about 5 phr, from about 0.1 phr to about 7.5 phr, from about 0.1 phr to about 10 phr, from about 1 phr to about 7.5 phr, from about 1 phr to about 10 phr, from about 5 phr to about 7.5 phr, from about 5 phr to about 10 phr, or from about 7.5 phr to about 10 phr.

In some embodiments, a Tire Composition of the Disclosure comprises about 3 phr of a second antidegradant. In some embodiments, the composition comprises about 0.001 phr, about 0.01 phr, about 0.1 phr, about 1 phr, about 2 phr, about 4 phr, about 5 phr, about 7.5 phr, or about 10 phr of a second antidegradant.

In some embodiments, the present disclosure provides a composition comprising a Compound or Composition of the Disclosure and one or more carriers. The term “carrier” as used herein refers to a solid that can adsorb a liquid while retaining the general properties of a solid at room temperature. In some embodiments, the carrier is an inert material. In some embodiments, the carrier has a high surface area. In some embodiments, the carrier comprises particles with diameters of less than 500 microns.

In some embodiments, the composition comprises from about 15 wt/wt % to about 85 wt/wt % of one or more carriers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt % from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt % from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt % from about 15 wt/wt % to about 25 wt/wt % from about 15 wt/wt % to about 35 wt/wt % from about 15 wt/wt % to about 45 wt/wt % from about 15 wt/wt % to about 55 wt/wt % from about 15 wt/wt % to about 65 wt/wt % from about 15 wt/wt % to about 75 wt/wt % from about 15 wt/wt % to about 95 wt/wt % from about 25 wt/wt % to about 35 wt/wt % from about 25 wt/wt % to about 45 wt/wt % from about 25 wt/wt % to about 55 wt/wt % from about 25 wt/wt % to about 65 wt/wt % from about 25 wt/wt % to about 75 wt/wt % from about 25 wt/wt % to about 85 wt/wt % from about 25 wt/wt % to about 95 wt/wt % from about 35 wt/wt % to about 45 wt/wt % from about 35 wt/wt % to about 55 wt/wt % from about 35 wt/wt % to about 65 wt/wt % from about 35 wt/wt % to about 75 wt/wt % from about 35 wt/wt % to about 85 wt/wt % from about 35 wt/wt % to about 95 wt/wt % from about 45 wt/wt % to about 55 wt/wt % from about 45 wt/wt % to about 65 wt/wt % from about 45 wt/wt % to about 75 wt/wt % from about 45 wt/wt % to about 85 wt/wt % from about 45 wt/wt % to about 95 wt/wt %, from about 55 wt/wt % to about 65 wt/wt % from about 55 wt/wt % to about 75 wt/wt % from about 55 wt/wt % to about 85 wt/wt % from about 55 wt/wt % to about 95 wt/wt % from about 65 wt/wt % to about 75 wt/wt % from about 65 wt/wt % to about 85 wt/wt %, from about 65 wt/wt % to about 95 wt/wt % from about 75 wt/wt % to about 85 wt/wt % from about 75 wt/wt % to about 95 wt/wt %, or from about 85 wt/wt % to about 95 wt/wt % of one or more carriers.

In some embodiments, the composition comprises about 15 wt/wt of one or more carriers. In some embodiments, the composition comprises about 1 wt/wt %, about 5 wt/wt % about 10 wt/wt %, about 20 wt/wt % about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt %, of one or more carriers.

In some embodiments, the present disclosure provides a composition recited in Table 9.

TABLE 9

| | Compo- | | | Compo- |
|---|---|---|---|---|
| | nent 1 | Composition | Compo- | nent 2 |
| Component 1 | wt/wt % | Type | nent 2 | wt/wt % |
| Compound or | 0.1-10 | Tire or other | Elastomer | 5-95 |
| Composition of | | rubber article | | |
| the Disclosure | | (belt, hose, | | |
| | | mount, etc) | | |
| | | (vulcanized) | | |
| Compound or | 0.01-10 | In-tire | Elastomer | 5-95 |
| Composition of | | component or | | |
| the Disclosure | | built up | | |
| | | component or | | |
| | | other end use | | |
| | | component | | |
| | | (tread, polymer | | |
| | | blends, etc) | | |
| | | (unvulcanized) | | |
| Compound or | 5-95 | Masterbatch | Elastomer | 95-5 |
| Composition of | | | | |
| the Disclosure | | | | |
| Compound or | 5-95 | Premix | Filler | 95-5 |
| Composition of | | | | |
| the Disclosure | | | | |
| Compound or | 5-95 | Premix | Rubber | 95-5 |
| Composition of | | | chemical | |
| the Disclosure | | | | |
| Compound or | 5-95 | Premix | Plasticizer | 95-5 |
| Composition of | | | | |
| the Disclosure | | | | |
| Compound or | 5-95 | Premix | Additional | 95-5 |
| Composition of | | | antidegradant(s) | |
| the Disclosure | | | | |

In some embodiments, the vulcanized tire or other rubber article composition of Table 9 comprises about 0.1 wt/wt %, about 0.2 wt/wt %, about 0.3 wt/wt %, about 0.4 wt/wt %, about 0.5 wt/wt %, about 0.6 wt/wt %, about 0.7 wt/wt %, about 0.8 wt/wt %, about 0.9 wt/wt %, about 1 wt/wt %, about 2 wt/wt %, about 3 wt/wt %, about 4 wt/wt %, about 5 wt/wt %, about 6 wt/wt %, about 7 wt/wt %, about 8 wt/wt %, about 9 wt/wt %, or about 10 wt/wt % of a Compound or Composition of the Disclosure. In some embodiments, the vulcanized tire or other rubber article of Table 9 comprises comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of at least one elastomer.

In some embodiments, the unvulcanized in-tire component or built up component or other end use component composition of Table 9 comprises about 0.01 wt/wt %, about 0.02 wt/wt %, about 0.03 wt/wt %, about 0.04 wt/wt %, about 0.05 wt/wt %, about 0.06 wt/wt %, about 0.07 wt/wt %, about 0.08 wt/wt %, about 0.09 wt/wt %, about 0.1 wt/wt %, about 0.2 wt/wt %, about 0.3 wt/wt %, about 0.4 wt/wt %, about 0.5 wt/wt %, about 0.6 wt/wt %, about 0.7 wt/wt % about 0.8 wt/wt %, about 0.9 wt/wt %, about 1 wt/wt %, about 2 wt/wt %, about 3 wt/wt % about 4 wt/wt % about 5 wt/wt %, about 6 wt/wt %, about 7 wt/wt %, about 8 wt/wt % about 9 wt/wt % or about 10 wt/wt % of a Compound or Composition of the Disclosure. In some embodiments, the unvulcanized in-tire component or built up component or other end use component composition of Table 9 comprises comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of at least one elastomer

The present disclosure also provides processes for preparing a composition comprising a Compound or Composition of the Disclosure and one or more carriers, the process comprising admixing the compound and the one or more carriers.

The present disclosure also provides lubricant compositions comprising a Compound or Composition of the Disclosure and a lubricant. Non-limiting examples of lubricants include mineral oil, higher molecular weight petroleum distillates such as aromatic, naphthenic, and paraffinic distillates, synthetic oils such as polyalpha-olefin (PAO), synthetic esters, polyalkylene glycols (PAG), phosphate esters, perfluoropolyether (PFPE), alkylated naphthlalenes (AN), silicate esters, ionic fluids, and multiply alkylated cyclopentanes (MAC), solid lubricants such as polytetrafluoroethylene (PTFE), graphite, hexagonal boron nitride, molybdenum disulfide, tungsten disulfide, aqueous lubricants such as hydrated brush polymers, and biolubricants such as triglyceride esters, high oleic canola oil, castor oil, palm oil, sunflower seed oil, and rapeseed oil.

The present disclosure also provides combustible fuel compositions comprising a combustible fuel and a Compound or Composition of the Disclosure. Non-limiting examples of combustible fuel include gasoline, diesel, kerosene, liquefied petroleum gas, synthetic fuel, and biodisesel.

The present disclosure also provides fuel additive compositions comprising a fuel additive and a Compound or Composition of the Disclosure. Non-limiting examples of fuel additives include oxygenates such as alcohols and ethers, antioxidants, stabilizers, detergents, antiknock agents, lead scavengers, fuel dyes, viscosity modifiers, and butyl rubber. In some embodiments, the butyl rubber is in the form of polyisobutylene succinimide. In some embodiments, the butyl rubber is added as a detergent to prevent fouling of diesel fuel injectors.

### IV. Vulcanized Elastomeric Articles

The present disclosure also provides vulcanized elastomeric articles comprising a Compound or Composition of the Disclosure. The term “vulcanized elastomeric article” refers to an article that is made by forming a composition comprising an elastomer into a specific shape and vulcanizing the composition to provide the article.

The present disclosure also provides vulcanized elastomeric articles prepared using a Tire Composition of the Disclosure.

In some embodiments, the vulcanized elastomeric article is a tire. In some embodiments, the tire is a passenger vehicle tire, a light truck tire, a heavy truck or bus tire, a motorcycle tire, an agriculture tire, an earthmover tire, an airplane tire, or a racing tire.

In some embodiments, the vulcanized elastomeric article is a component of a tire. In some embodiments, the component is a bead, a belt, a body ply, an inner liner, a sidewall, an undertread, or a tread.

In some embodiments, the vulcanized elastomeric article is a rubber overshoe, a sealing strip, an acoustic panel, an air spring, a bellow, a membrane, a tactile sensor, a crash pad, a hose, a conveyor belt, or a flooring.

In some embodiments, the present disclosure provides a vehicle comprising a vulcanized elastomeric article comprising a Compound or Composition of the Disclosure.

### V. Processes

The present disclosure also provides processes for preparing a vulcanized elastomeric article, the process comprising:
(a) forming a Tire Composition of the Disclosure into a formed shape; and
(b) vulcanizing the formed shape,
to provide a vulcanized elastomeric article.

In some embodiments, the vulcanizing is performed at an average temperature of from about 140° C. to about 160° C. In some embodiments, the vulcanizing is performed at an average temperature of from about 80° C. to about 100° C., from about 80° C. to about 120° C., from about 80° C. to about 140° C., from about 80° C. to about 160° C., from about 80° C. to about 180° C., from about 80° C. to about 200° C., from about 100° C. to about 120° C., from about 100° C. to about 140° C., from about 100° C. to about 160° C., from about 100° C. to about 180° C., from about 100° C. to about 200° C., from about 120° C. to about 140° C., from about 120° C. to about 160° C., from about 120° C. to about 180° C., from about 120° C. to about 200° C., from about 140° C. to about 180° C., from about 140° C. to about 200° C., from about 160° C. to about 180° C., from about 160° C. to about 200° C., or from about 180° C. to about 200° C.

In some embodiments, the vulcanizing is performed at an average temperature of about 150° C. In some embodiments, the vulcanizing is performed at an average temperature of about 80° C., about 100° C., about 120° C., about 140° C., about 160° C., about 180° C., or about 200° C.

The present disclosure also provides processes for retreading tires, the process comprising:
(a) applying a Tire Composition of the Disclosure to a tire;
(b) disposing a pre-vulcanized tread around the tire;
(c) disposing a curing envelope around the tire; and
(d) vulcanizing the tire.

In another embodiment, the present disclosure provides a process for preparing the composition comprising Compound (10), or a salt or solvate thereof.

In another embodiment, the present disclosure provides a process for preparing the composition comprising Compound (10), or a salt or solvate thereof, and Compound (2), Compound (4), and/or Compound (11), the process comprising reacting 4aminodiphenylamine with acetone, 2,2-dimethoxypropane, or mesityl oxide in the presence of a strong acid, e.g., hydrochloric acid.

The present disclosure provides the following particular process embodiments.

Process Embodiment 1. A process for preparing the composition of any one of Composition Embodiments 1-112, see above, the process comprising reacting a composition comprising 4-aminodiphenylamine with acetone, 2,2-dimethoxypropane, diacetone alcohol, or mesityl oxide in the presence of a catalyst.

Process Embodiment 2. The process of Process Embodiment 1, wherein the catalyst is a homogeneous catalyst.

Process Embodiment 3. The process of Process Embodiment 2, wherein the homogeneous catalyst is a Lewis acid, a Bronsted acid, a base, a halogen, a halogen donor, or a combination thereof.

Process Embodiment 4. The process of Process Embodiment 2, wherein the homogeneous catalyst is selected from the group consisting of iodine, pyridinium perbromide, bromine, bismuth(III) trifluoromethane sulfonate, zinc(II) trifluoromethane sulfonate, hydrobromic acid, magnesium(II) bromide, aluminum(III) chloride, p-toluene sulfonic acid monohydrate, N-bromosuccinimide, copper(II) bromide, copper(II) chloride, methane sulfonic acid, benzene sulfonic acid, boron trifluoride diethyl etherate, boron trifluoride dihydrate, hydrochloric acid, copper(II) trifluoromethane sulfonate, iron(III) chloride, magnesium(II) chloride, copper(I) iodide, and zinc(II) chloride, or the salts or solvates thereof.

Process Embodiment 5. The process of Process Embodiment 4, wherein the homogeneous catalyst is selected from the group consisting of pyridinium perbromide, magnesium(II) bromide, magnesium(II) bromide hexahydrate, and aluminum(III) chloride.

Process Embodiment 6. The process of Process Embodiment 1, wherein the catalyst is a heterogeneous catalyst.

Process Embodiment 7. The process of Process Embodiment 6, wherein the heterogeneous catalyst is an acid treated resin, a solid-supported acid, a composite catalyst, a solid acid, or a base catalyst.

Process Embodiment 8. The process of Process Embodiment 7, wherein the acid treated resin is a polyfluorinated sulfonic acid resin, a strongly acidic sulfonic acid exchange resin (Amberlyst 15), a macroporous strong acid ion exchange resin, a strong acid ion exchange resin, a solid acidic ion exchange resin (NRW150), a sifted strong acidic ion exchange resin, or a strong acidic ion exchange resin (type A2 acidic resin).

Process Embodiment 9. The process of Process Embodiment 7, wherein the solid-supported acid is micro-meso-macroporous zeolite catalyst H-Y-MMM, strong-acidity molecular sieve H-MCM-22, an organic protonic acid supported by graphene oxide, an organic protonic acid supported by a zeolite, a small pore size zeolite, a heterogeneous acidic silica catalyst, Zn²⁺, Sn²⁺, and Cu²⁺ exchanged tungstophosphoric acid (TPA) supported on γ-Al₂O₃, tonsil activated earth (acid treated bentonite clay), alumina, titania, zirconia, niobia, silica-alumina, sulfated zirconia, tungstated zirconia, para-toluenesulfonic acid (p-TSA)/ZnCl₂, or WO₃/activated carbon/SO₃H.

Process Embodiment 10. The process of Process Embodiment 7, wherein the composite catalyst is zirconium doped titania composite oxide catalyst, H₂SO₄—MoO₃—TiO₂ solid superacid, or p-toluenesulfonic acid/iodine.

Process Embodiment 11. The process of Process Embodiment 7, wherein the solid acid is a heteropolyacid or a metal-organic framework.

Process Embodiment 12. The process of Process Embodiment 7, wherein the base catalyst is ZrO₂/MgO.

Process Embodiment 13. The process of any one of Process Embodiments 1-12 comprising reacting a composition comprising 4-aminodiphenylamine with acetone.

Process Embodiment 14. The process of any one of Process Embodiments 1-12 comprising reacting a composition comprising 4-aminodiphenylamine with 2,2-dimethoxypropane.

Process Embodiment 15. The process of any one of Process Embodiments 1-12 comprising reacting a composition comprising 4-aminodiphenylamine with diacetone alcohol.

Process Embodiment 16. The process of any one of Process Embodiments 1-12 comprising reacting a composition comprising 4-aminodiphenylamine with mesityl oxide.

### VI. Kits

The present disclosure also provides kits comprising Compound or Composition of the Disclosure or a Tire Composition of the Disclosure packaged in a manner, e.g., in a container, that facilitates use of the compound or composition to practice the processes and/or methods of the present disclosure. In some embodiments, the kit comprises a Tire Composition of the Disclosure and instructions for using the composition in a vulcanizable elastomeric composition. In some embodiments, the kit comprises a Tire Composition of the Disclosure and instructions for using the composition to prepare a vulcanized elastomeric article. The composition may be packaged in any suitable container, such as a sealed bottle or vessel, with a label affixed to the container or included in the kit that describes the composition and proper use thereof.

### VII. Definitions

The term “weight ratio” as used herein refers to mass of one compound divided by the mass of another compound in a composition. For example, the weight ratio of Compound (2) to Compound (10) in a composition comprising 1 gram of Compound (2) and 1000 grams of Compound (10) is 0.001. The weight ratio of Compound (2) to Compound (10) in a composition comprising 10 grams of Compound (2) and 100 grams of Compound (10) is 0.1.

In some embodiments, a compound of the present disclosure comprises a mixture of E and Z stereoisomers as illustrated for Compound (4) below:

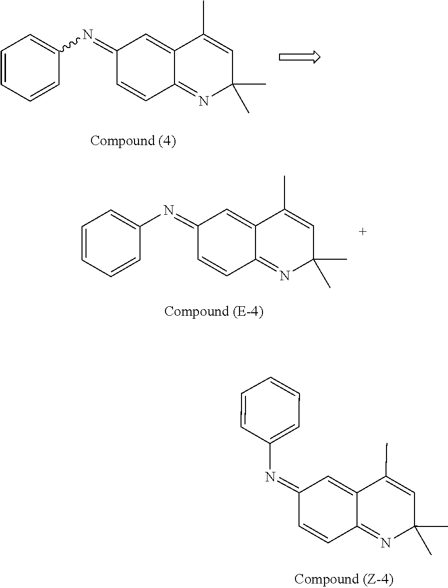 [SMILES: CC1=CC(C)(C)N=C2C=CC(=Nc3ccccc3)C=C12 ; CC1=CC(C)(C)N=C2C=C/C(=N\c3ccccc3)C=C12 ; CC1=CC(C)(C)N=C2C=C/C(=N/c3ccccc3)C=C12]

In some embodiments, a compound of the present disclosure comprises comprises a mixture of E and Z stereoisomers, wherein the E:Z ratio is 500:1 to 1:500. In some embodiments, the E:Z ratio is 100:1 to 1:100. In some embodiments, the E:Z ratio is 10:1 to 1:10. In some embodiments, the E:Z ratio is 5:1 to 1:5. In some embodiments, the E:Z ratio is 3:1 to 1:3. In some embodiments, the E:Z ratio is 2:1 to 1:2. In some embodiments, the E:Z ratio is about 10:1, about 9:1, about 8:1, about 7:1, about 6:1, about 5:1, about 4:1, about 3:1, or about 2:1. In some embodiments, the E:Z ratio is about 1:10, about 1:9, about 1:8, about 1:7, about 1:6, about 1:5, about 1:4, about 1:3, or about 1:2. In some embodiments, the E:Z ratio is about 1:1.

In some embodiments, a Compound of the Disclosure comprises only the E stereoisomer, i.e., there is no detectable amount of the Z stereoisomer as measured by HPLC.

In some embodiments, a Compound of the Disclosure comprises only the Z stereoisomer, i.e., there is no detectable amount of the E stereoisomer as measured by HPLC.

The term “about,” as used herein, includes the recited number±10%. Thus, “about 10” means 9 to 11.

The word “comprising” is used in a manner consistent with its open-ended meaning, that is, to mean that a given product, composition, or process can optionally also have additional features or elements beyond those expressly described. It is understood that wherever embodiments are described with the language “comprising,” otherwise analogous embodiments described in terms of “consisting of” and/or “consisting essentially of” are also contemplated and within the scope of this disclosure.

Salts and solvates, e.g., hydrates, of the Compounds of the Disclosure can also be used in the methods disclosed herein.

The present disclosure encompasses the preparation and use of salts of Compounds of the Disclosure. Salts of Compounds of the Disclosure can be prepared during the final isolation and purification of the compounds or separately by reacting the compound with an acid having a suitable cation. Salts of Compounds of the Disclosure can be acid addition salts formed with acceptable acids. Examples of acids which can be employed to form salts include inorganic acids such as nitric, boric, hydrochloric, hydrobromic, sulfuric, and phosphoric, and organic acids such as oxalic, maleic, succinic, and citric. Non-limiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and ptoluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to Compounds of the Disclosure appearing herein is intended to include Compounds of the Disclosure as well as salts, hydrates, or solvates thereof.

The present disclosure encompasses the preparation and use of solvates of Compounds of the Disclosure. The term “solvate” as used herein is a combination, physical association and/or solvation of a compound of the present disclosure with a solvent molecule such as, e.g., a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a solvent, such as water, methanol, and ethanol, and it is intended that the disclosure includes both solvated and unsolvated forms of Compounds of the Disclosure.

One type of solvate is a hydrate. A “hydrate” relates to a particular subgroup of solvates where the solvent molecule is water. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a Compound or Composition of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

As used herein, the term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or B; A or C; B or C; A and B; A and C; B and C; A (alone); B (alone); and C (alone).

The term “wt/wt %” as used herein refers to the mass of one component in a composition or blend, e.g., a composition comprising a Compound of the Disclosure and one or more elastomers; or a Composition of the Disclosure and one or more fillers; or a blend comprising two or more elastomers, etc., divided by the combined mass of all components in the composition or blend, times 100. For example, the wt/wt % of a Compound of the Disclosure in a composition comprising 1 kg of the compound, 1 kg natural rubber, and 2 kg of synthetic rubber, is 25 wt/wt % (1 kg/4 kg=0.25×100=25 wt/wt %). The wt/wt % of a Compound of the Disclosure in a composition comprising 1 kg of the compound and 1 kg of carbon black is 50 wt/wt % (1 kg/2 kg=0.20×100=50 wt/wt %). The wt/wt % of a Compound of the Disclosure in a composition comprising 1 kg of the compound, 1 kg natural rubber, 2 kg of synthetic rubber, and 1 kg of carbon black is 20 wt/wt % (1 kg/5 kg=0.20×100=20 wt/wt %). The wt/wt % of natural rubber in a blend of one or more elastomers comprising 20 kg natural rubber and 30 kg synthetic rubber is 40 wt/wt % (20 kg/50 kg=0.40×100=40 wt/wt %).

As used herein, the term “masterbatch” refers to a composition comprising at least one elastomer and a Compound or Composition of the Disclosure. In some embodiments, the masterbatch comprises from about 5 wt/wt % to about 95 wt/wt % of at least one elastomer and from about 5 wt/wt % to about 95 wt/wt % of a Compound or Composition of the Disclosure. In some embodiments, the masterbatch comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of a Compound or Composition of the Disclosure. In some embodiments, the masterbatch comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of at least one elastomer.

As used herein, the term “premix” refers to a composition comprising a Compound or Composition of the Disclosure and at least one additional component, e.g., a filler, a rubber chemical, a plasticizer, an additional antidegradant, or a combination thereof. In some embodiments, the premix does not comprise an elastomer. In some embodiments, the premix comprises from about 5 wt/wt % to about 95 wt/wt % of a Compound or Composition of the Disclosure and from about 5 wt/wt % to about 95 wt/wt % of the least one additional component, e.g., a filler, a rubber chemical, a plasticizer, an additional antidegradant, or a combination thereof. In some embodiments, the premix comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of a Compound of the Disclosure. In some embodiments, the premix comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of at least one additional component, e.g., a filler, a rubber chemical, a plasticizer, an additional antidegradant, or a combination thereof.

The term “one or more” as used herein is intended to mean that at least one component in a relevant category of components, e.g. one or more elastomers, is present and, in some embodiments, more than one component is present. In some embodiments, one or more means 1 to 50. In some embodiments, one or more means 1 to 40. In some embodiments, one or more means 1 to 30. In some embodiments, one or more means 1 to 20. In some embodiments, one or more means 1 to 10. In some embodiments, one or more means 1 to 5. In some embodiments, one or more means 1 to 3. In some embodiments, one or more means 1 or 2. In some embodiments, one or more means 1.

### VIII. Particular Embodiments

The present disclosure provides the following particular embodiments. The compound structures are provided in Table 1.

Embodiment 1. Compound (9), or a salt or solvate thereof.

Embodiment 2. Compound (10), or a salt or solvate thereof.

Embodiment 3. A composition comprising the compound of Embodiments 1 or 2, and Compound (1), wherein the composition comprises about 0.0001% to about 10% of Compound (1) as measured by HPLC analysis, e.g., about 0.001% to about 10%, about 0.01% to about 10%, about 0.1% to about 10%, about 0.0001% to about 5%, about 0.001% to about 5%, about 0.01% to about 5%, about 0.1% to about 5%, about 0.0001% to about 1%, about 0.001% to about 1%, about 0.01% to about 1%, about 0.01% to about 0.5%, about 0.05% to about 0.5%, or about 0.1% to 0.5% of Compound (1).

Embodiment 4. A composition comprising the compound of Embodiments 1 or 2, and Compound (2), wherein the composition comprises about 0.0001% to about 10% of Compound (2) as measured by HPLC analysis, e.g., about 0.001% to about 10%, about 0.01% to about 10%, about 0.1% to about 10%, about 0.0001% to about 5%, about 0.001% to about 5%, about 0.01% to about 5%, about 0.1% to about 5%, about 0.0001% to about 1%, about 0.001% to about 1%, about 0.01% to about 1%, about 0.01% to about 0.5%, about 0.05% to about 0.5%, or about 0.1% to 0.5% of Compound (2).

Embodiment 5. A composition comprising the compound of Embodiments 1 or 2, and Compound (3), wherein the composition comprises about 0.001% to about 10% of Compound (3) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (3).

Embodiment 6. A composition comprising the compound of Embodiments 1 or 2, and Compound (4), wherein the composition comprises about 0.001% to about 10% of Compound (4) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (4).

Embodiment 7. A composition comprising the compound of Embodiments 1 or 2, and Compound (5), wherein the composition comprises about 0.001% to about 10% of Compound (5) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (5).

Embodiment 8. A composition comprising the compound of Embodiments 1 or 2, and Compound (6), wherein the composition comprises about 0.001% to about 10% of Compound (6) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (6).

Embodiment 9. A composition comprising the compound of Embodiments 1 or 2, and Compound (7), wherein the composition comprises about 0.001% to about 10% of Compound (7) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (7).

Embodiment 10. A composition comprising the compound of Embodiments 1 or 2, and Compound (8), wherein the composition comprises about 0.001% to about 10% of Compound (8) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (8).

Embodiment 11. A composition comprising the compound of Embodiments 1 or 2, and Compound (11A), wherein the composition comprises about 0.001% to about 10% of Compound (11A) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (11A).

Embodiment 12. A composition comprising the compound of Embodiments 1 or 2, and Compound (12), wherein the composition comprises about 0.001% to about 10% of Compound (12) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (12).

Embodiment 13. A composition comprising the compound of Embodiments 1 or 2, and Compound (13), wherein the composition comprises about 0.001% to about 10% of Compound (13) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (13).

Embodiment 14. A composition comprising the compound of Embodiments 1 or 2, and Compound (14), wherein the composition comprises about 0.001% to about 10% of Compound (14) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (14).

Embodiment 15. A composition comprising the compound of Embodiments 1 or 2, and Compound (15), wherein the composition comprises about 0.001% to about 10% of Compound (15) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (15).

Embodiment 16. A composition comprising the compound of Embodiments 1 or 2, and Compound (16), wherein the composition comprises about 0.001% to about 10% of Compound (16) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (16).

Embodiment 17. A composition comprising the compound of Embodiments 1 or 2, and Compound (17), wherein the composition comprises about 0.001% to about 10% of Compound (17) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (17).

Embodiment 18. A composition comprising the compound of Embodiments 1 or 2, and Compound (18), wherein the composition comprises about 0.001% to about 10% of Compound (18) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (18).

Embodiment 19. A composition comprising the compound of Embodiments 1 or 2, and Compound (19), wherein the composition comprises about 0.001% to about 10% of Compound (19) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (19).

Embodiment 20. A composition comprising the compound of Embodiments 1 or 2, and Compound (20A), wherein the composition comprises about 0.001% to about 20% of Compound (20A) as measured by HPLC analysis, e.g., about 0.01% to about 20%, about 0.1% to about 20%, about 0.001% to about 15%, about 0.01% to about 15%, about 0.1% to about 15%, about 0.001% to about 10%, about 0.01% to about 10%, about 0.01% to about 15%, about 0.5% to about 15%, or about 1% to 15% of Compound (20).

Embodiment 21. A composition comprising Compound (10); and:
(i) Compound (2), wherein the composition comprises about 0.0001% to about 10% of Compound (2) as measured by HPLC analysis, e.g., about 0.01% to about 10%, about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (2); and/or
(ii) Compound (4), wherein the composition comprises about 0.001% to about 10% of Compound (4) as measured by HPLC analysis, e.g., about 0.01% to about 10%, about 0.1o % to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (4); and/or.
(iii) Compound (11A) or Compound (11B), wherein the composition comprises about 0.001% to about 10% of Compound (11A) or Compound (11B) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (11A) or Compound (11B); and/or
(iv) Compound (20A) or Compound (20B), wherein the composition comprises about 0.001% to about 10% of Compound (20A) or Compound (20B) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (20A) or Compound (20B)

Embodiment 22. Compound (3).

Embodiment 23. Compound (4).

Embodiment 24. A composition comprising the compound of Embodiments 22 or 23.

Embodiment 25. A composition comprising the composition of any one of Embodiments 3-21, 24, 118-125, 127, 129, 130, 133, or 134, or the compound of Embodiments 22, 23, 126, 128, 131, or 132; and:
(i) one or more elastomers; or
(ii) one or more fillers; or
(iii) one or more rubber chemicals; or
(iv) one or more plasticizers; or
(v) one or more second antidegradants; or
(vi) a combination of one or more elastomers, one or more fillers, one or more rubber chemicals, one or more plasticizers, and/or one or more second antidegradants.

Embodiment 26. The composition of claim 25, wherein the composition comprises one or more elastomers.

Embodiment 27. The composition of claim 26, wherein the composition comprises from about 15 wt/wt % to about 85 wt/wt % of the composition of (i) and from about 15 wt/wt % to about 85 wt/wt % of the one or more elastomers.

Embodiment 28. The composition of any one of Embodiments 25-27, wherein the one or more elastomers comprise natural rubber (NR).

Embodiment 29. The composition of Embodiment 28, wherein the one or more elastomers comprises from about 5 wt/wt % to about 80 wt/wt % natural rubber (NR).

Embodiment 30. The composition of Embodiment 28 or 29, wherein the natural rubber comprises rubber derived from an alternative rubber plant.

Embodiment 31. The composition of Embodiment 30, wherein the alternative rubber plant is Parthenium argentatum (guayule) or Taraxacum kok-saghyz (Russian dandelion).

Embodiment 32. The composition of any one of Embodiments 26-31, wherein the one or more elastomers comprise synthetic rubber.

Embodiment 33. The composition of Embodiment 32, wherein the synthetic rubber comprises an unsaturated rubber, a saturated rubber, a rubber with fluoro and fluoralkyl or fluoralkoxy substituent groups on the polymer chain (FKM), a silicone rubber (Q), or a blend thereof.

Embodiment 34. The composition of Embodiment 33, wherein the unsaturated rubber comprises polyisoprene rubber (IR), butyl rubber (IIR), polybutadiene rubber (BR), styrene butadiene rubber (SBR), nitrile butadiene rubber (NBR), chloroprene rubber (CR), ethylene propylene diene rubber (EPDM), or a blend thereof.

Embodiment 35. The composition of Embodiment 34, wherein the unsaturated rubber comprises styrene butadiene rubber (SBR).

Embodiment 36. The composition of Embodiment 35, wherein the one or more elastomers comprise from about 5 wt/wt % to about 80 wt/wt % styrene butadiene rubber (SBR).

Embodiment 37. The composition of any one of Embodiments 34-36, wherein the unsaturated rubber comprises polybutadiene rubber (BR).

Embodiment 38. The composition of Embodiment 37, wherein the one or more elastomers comprise from about 5 wt/wt % to about 80 wt/wt % polybutadiene rubber (BR).

Embodiment 39. The composition of any one of Embodiments 34-38, wherein the unsaturated rubber comprises butyl rubber (IIR).

Embodiment 40. The composition of Embodiment 39, wherein the one or more elastomers comprise from about 1 wt/wt % to about 30 wt/wt % butyl rubber (IIR).

Embodiment 41. The composition of any one of Embodiments 33-40, wherein the saturated rubber comprises acrylic rubber (ACM), chlorinated polyethylene (CM), chlorosulfonated polyethylene (CSM), polychloromethyloxiran (CO), ethylene-ethyl acrylate copolymer (EAM), epichlorohydrin rubber (ECO), ethylene propylene rubber (EPM), ethylenevinylacetate copolymer (EVM), or a blend thereof.

Embodiment 42. The composition of Embodiments any one of Embodiments 35-41, wherein the one or more elastomers further comprises recycled rubber.

Embodiment 43. The composition of any one of Embodiments 35-42, wherein the composition comprises from about 0.1 phr to about 10 phr of the composition of (i).

Embodiment 44. The composition of Embodiment 43, wherein the composition comprises from about 0.5 phr to about 5 phr of the composition of (i).

Embodiment 45. The composition of Embodiment 44, wherein the composition comprises from about 1 phr to about 5 phr of the composition of (i).

Embodiment 46. The composition of any one of Embodiments 25-45, wherein the composition comprises one or more fillers.

Embodiment 47. The composition of Embodiment 46, wherein the composition comprises from about 15 wt/wt % to about 85 wt/wt % of the composition of (i) and from about 15 wt/wt % to about 85 wt/wt % of the one or more fillers.

Embodiment 48. The composition of Embodiments 46 or 47, wherein the composition comprises from about 30 phr to about 500 phr of one or more fillers.

Embodiment 49. The composition of any one of Embodiments 46-48, wherein the one or more fillers comprise carbon black, silica, kaolin, calcium silicate, talc, carbon nanotubes (CNT), carbon fibers (HCF), graphite, graphenes, aluminosilicates, starch, fibers, or a combination thereof.

Embodiment 50. The composition of Embodiment 49, wherein the one or more fillers comprise silica.

Embodiment 51. The composition of Embodiment 50, wherein the silica is derived from rice husks.

Embodiment 52. The composition of any one of Embodiments 46-51, wherein the one or more fillers comprise carbon black.

Embodiment 53. The composition of any one of Embodiments Embodiment 25-52, wherein the composition comprises one or more rubber chemicals.

Embodiment 54. The composition of Embodiment 53, wherein the composition comprises from about 15 wt/wt % to about 85 wt/wt % of the composition of (i) and from about 15 wt/wt % to about 85 wt/wt % of one or more rubber chemicals.

Embodiment 55. The composition of Embodiments 53 or 54, wherein the composition comprises from about 0.1 phr to about 30 phr of one or more rubber chemicals.

Embodiment 56. The composition of Embodiment 55, wherein the composition comprises from about 1 phr to about 20 phr of one or more rubber chemicals.

Embodiment 57. The composition of any one of Embodiments 53-56, wherein the one or more rubber chemicals comprise one or more vulcanizing agents, one or more accelerators, one or more activators, one or more pre-vulcanization inhibitors, or a combination thereof.

Embodiment 58. The composition of Embodiment 57, wherein the one or more rubber chemicals comprise one or more vulcanizing agents.

Embodiment 59. The composition of Embodiment 58, wherein the one or more vulcanizing agents comprise sulfur, peroxide, resin, or a combination thereof.

Embodiment 60. The composition of Embodiment 59, wherein the sulfur is octasulfur (S8), cyclododecasulfur (Si₂), polymeric sulfur, or a combination thereof.

Embodiment 61. The composition of Embodiment 59, wherein the peroxide is benzoyl peroxide, dicumyl peroxide (DC), 2,5-dimethyl-2,5-di-(tert-butylperoxy)-3-hexyne (2,5 Tri), 2,5-dimethyl-2,5-di(tert-butylperoxy)hexane (DDPH), di-(2-tert-butylperoxyisopropyl)benzene (VC), butyl-4,4-di-(tert-butylperoxy)valerate (VAL), 1,1-di(tert-butylperoxy)-3,3,5-trimethylcyclohexane (TMC), or a combination thereof.

Embodiment 62. The composition of Embodiment 59, wherein the resin is a bonding resin.

Embodiment 63. The composition of Embodiments 57-62, wherein the one or more rubber chemicals comprise one or more accelerators.

Embodiment 64. The composition of Embodiment 63, wherein the one or more accelerators comprise a guanidine, a thiazole, a sulfenamide, a thiuram, a dithiocarbamate, a xanthate, a thiophosphate, or a combination thereof.

Embodiment 65. The composition of Embodiment 64, wherein the guanidine is diphenylguanidine (DPG).

Embodiment 66. The composition of Embodiment 64, wherein the thiazole comprises 2mercaptobenzothiazole (MBT), zinc 2mercaptobenzothiazole (ZMBT), mercaptobenzothiazole disulfide (MBTS), N-tert-butyl-2-benzothiazole sulfenimide (TBSI), or a combination thereof.

Embodiment 67. The composition of Embodiment 64, wherein the sulfenamide comprises N-tert-butyl-2-benzothiazylsulfenamide (TBBS), N-cyclohexylbenzothiazol-2-sulfenamide (CBS), dicyclohexyl-2-benzothiazolesulfenamide (DCBS), N-oxydiethylene benzothiazole sulfenamide (OBTS), N-oxydiethylenethiocarbamyl-N′-oxydiethylene sulfenamide (OTOS), thiocarbamyl sulfenamide, or a combination thereof.

Embodiment 68. The composition of Embodiment 64, wherein the thiuram is dimethylcarbamothioic dithioperoxyanhydride (thiram), dipentamethylene thiuram tetrasulfide (DPIT), tetrabenzyl thiuram disulfide (TBzTD), tetraethylthiuram disulfide (TETD), tetramethylthiuram disulfide (TMTD), tetramethylthiuram monosulfide (TMTM), or a combination thereof.

Embodiment 69. The composition of Embodiment 64, wherein the dithiocarbamate comprises zinc dimethyldithiocarbamate (ZDMC), zinc diethyldithiocarbamate (ZDEC), zinc dibutyldithiocarbamate (ZDBC), nickel dibutyldithiocarbamate (NDBC), sodium dibenzyldithiocarbamate (SBEC), sodium diethyldithiocarbamate (SDEC), tellurium diethyldithiocarbamate (TDEC), zinc dibenzyldithiocarbamate (ZEBC), or a combination thereof.

Embodiment 70. The composition of any one of Embodiments 57-69, wherein the one or more rubber chemicals comprise one or more activators.

Embodiment 71. The composition of Embodiment 70, wherein the one or more activators comprise a metal oxide, an acid, a metal complex, or a combination thereof.

Embodiment 72. The composition of Embodiment 71, wherein the metal oxide comprises zinc oxide, magnesium oxide, lead oxide, or a combination thereof.

Embodiment 73. The composition of Embodiment 72, wherein the acid comprises stearic acid, lauric acid, or a combination thereof.

Embodiment 74. The composition of Embodiment 72, wherein the metal complex comprises zinc ethylhexanoate.

Embodiment 75. The composition of any one of Embodiments 57-74, wherein the one or more rubber chemicals comprise one or more pre-vulcanization inhibitors.

Embodiment 76. The composition of Embodiment 75, wherein the one or more pre-vulcanization inhibitors comprise N-(cyclohexylthio)phthalimide (CTP), benzoic anhydride, salicylic anhydride, phthalic anhydride, or a combination thereof.

Embodiment 77. The composition of any one of Embodiments 25-76, wherein the composition comprises one or more plasticizers.

Embodiment 78. The composition of Embodiment 77, wherein the composition comprises from about 15 wt/wt % to about 85 wt/wt % of the composition of (i) and from about 15 wt/wt % to about 85 wt/wt % of one or more plasticizers.

Embodiment 79. The composition of Embodiments 77 or 78, wherein the composition comprises from about 0.1 phr to about 30 phr of one or more plasticizers.

Embodiment 80. The composition of Embodiment 79, wherein the composition comprises from about 1 phr to about 20 phr of one or more plasticizers.

Embodiment 81. The composition of any one of Embodiments 77-80, wherein the one or more plasticizers comprise a mineral oil, an organic ester, a resin, a wax, an ester plasticizer, a naturally derived oil, or a combination thereof.

Embodiment 82. The composition of Embodiment 81, wherein the mineral oil is naphthenic oil, paraffinic oil, or aromatic oil.

Embodiment 83. The composition of Embodiment 81, wherein the naturally derived oil is soybean oil, vegetable oil, or orange oil.

Embodiment 84. The composition of any one of Embodiments 25-83, wherein the composition further comprises a second antidegradant.

Embodiment 85. The composition of Embodiment 84, wherein the composition comprises from about 15 wt/wt % to about 85 wt/wt % of the composition of (i) and from about 15 wt/wt % to about 85 wt/wt % of the one or more second antidegradants.

Embodiment 86. The composition of Embodiment 84 or 85, wherein the one or more second antidegradants is present in an amount of from about 0.001 phr to about 10 phr.

Embodiment 87. The composition of Embodiment 86, wherein the one or more second antidegradants is present in an amount of from about 0.1 phr to about 5 phr.

Embodiment 88. The composition of Embodiment 87, wherein the one or more second antidegradants is present in an amount of from about 0.5 phr to about 5 phr.

Embodiment 89. The composition of Embodiment 88, wherein the one or more second antidegradants is present in an amount of from about 1 phr to about 5 phr.

Embodiment 90. The composition of any one of Embodiments 84-89, wherein the one or more second antidegradants is an antioxidant.

Embodiment 91. The composition of any one of Embodiments 84-89, wherein the one or more second antidegradants is an antiozonant.

Embodiment 92. The composition of any one of Embodiments 84-91, wherein the one or more second antidegradants is a paraphenylenediamine (PPD), a trimethyldihydroquinoline (TMQ), a phenolic, an alkylated diphenylamine (DPA), or a diphenylamine-ketone condensate.

Embodiment 93. The composition of Embodiment 92, wherein the PPD is N′-(4-methylpentan-2-yl)-N⁴-phenylbenzene-1,4-diamine (6PPD), N-(1,4-dimethylpentyl)-N′-phenyl-p-phenylenediamine (7PPD), N′-phenyl-N⁴-(propan-2-yl)benzene-1,4-diamine (IPPD), N,N′-di-sec-butyl-p-phenylenediamine (44PD), N,N′-bis(1,3-dimethylbutyl)-p-phenylenediamine (66PD), N,N′-bis(1,4-dimethylpentyl)-p-phenylenediamine (77PD), or N—N′-dioctyl-p-phenylenediamine (88PD).

Embodiment 94. The composition of Embodiment 93, wherein the PPD is 6PPD.

Embodiment 95. The composition of Embodiment 92, wherein the TMQ is 2,2,4-trimethyl-1,2-dihydroquinoline or an oligomer or polymer thereof.

Embodiment 96. A vulcanized elastomeric article comprising the composition of any one of Embodiments 2595.

Embodiment 97. A vulcanized elastomeric article prepared using the composition of any one of Embodiments 25-95.

Embodiment 98. The vulcanized elastomeric article of Embodiments 96 or 97, wherein the vulcanized elastomeric article is a tire.

Embodiment 99. The vulcanized elastomeric article of Embodiment 98, wherein the tire is a passenger vehicle tire, a light truck tire, a heavy truck or bus tire, a motorcycle tire, an agriculture tire, an earthmover tire, an airplane tire, or a racing tire.

Embodiment 100. The vulcanized elastomeric article of Embodiments 96 or 97, wherein the vulcanized elastomeric article is a component of a tire.

Embodiment 101. The vulcanized elastomeric article of Embodiment 100, wherein the component is a bead, a belt, a body ply, an inner liner, a sidewall, an undertread, or a tread.

Embodiment 102. The vulcanized elastomeric article of Embodiments 96 or 97, wherein the vulcanized elastomeric article is a rubber overshoe, a sealing strip, an acoustic panel, an air spring, a bellow, a membrane, a tactile sensor, a crash pad, a hose, a conveyor belt, or a flooring.

Embodiment 103. A process for preparing a vulcanized elastomeric article, the process comprising:
(a) forming the composition of any one of Embodiments 25-95 into a formed shape; and
(b) vulcanizing the formed shape to provide a vulcanized elastomeric article.

Embodiment 104. The process of Embodiment 103, wherein the vulcanizing is performed at an average temperature of from about 120° C. to about 180° C.

Embodiment 105. The process of Embodiment 104, wherein the vulcanizing is performed at an average temperature of from about 140° C. to about 160° C.

Embodiment 106. The process of any one of Embodiments 103-105, wherein the vulcanized elastomeric article is a tire.

Embodiment 107. The process of Embodiment 106, wherein the tire is a passenger vehicle tire, a light truck tire, a heavy truck or bus tire, a motorcycle tire, an agriculture tire, an earthmover tire, an airplane tire, or a racing tire.

Embodiment 108. The process of any one of Embodiments 103-107, wherein the vulcanized elastomeric article is a component of a tire.

Embodiment 109. The process of Embodiment 108, wherein the component is a bead, a belt, a body ply, an inner liner, a sidewall, an undertread, or a tread.

Embodiment 110. The process of any one of Embodiments 103-105, wherein the vulcanized elastomeric article is a rubber overshoe, a sealing strip, an acoustic panel, an air spring, a bellow, a membrane, a tactile sensor, a crash pad, a hose, a conveyor belt, or a flooring.

Embodiment 111. A lubricant composition comprising a lubricant and the composition of any one of Embodiments 25-95.

Embodiment 112. A combustible fuel composition comprising a combustible fuel and the composition of any one of Embodiments 25-95.

Embodiment 113. A fuel additive composition comprising a fuel additive and the composition of any one of Embodiments 25-95.

Embodiment 114. A process for retreading tires, the process comprising:
(a) applying the composition of any one of Embodiments 25-95 to a tire;
(b) disposing a pre-vulcanized tread around the tire;
(c) disposing a curing envelope around the tire; and
(d) vulcanizing the tire.

Embodiment 115. A kit comprising the composition of any one of Embodiments 25-95 and instructions for using the composition in a vulcanizable elastomeric composition.

Embodiment 116. A kit comprising the composition of any one of Embodiments 25-95 and instructions for using the composition to prepare a vulcanized elastomeric article.

Embodiment 117. A process for preparing the composition of any one of Embodiments 3-21, the process comprising reacting 4-aminodiphenylamine with acetone, 2,2-dimethoxypropane, or mesityl oxide in the presence of a strong acid, e.g., hydrochloric acid.

Embodiment 118. A composition comprising the compound of Embodiments 1 or 2, and Compound (21), wherein the composition comprises about 0.0001% to about 10% of Compound (21) as measured by HPLC analysis, e.g., about 0.001% to about 10%, 0.01% to about 10%, about 0.1% to about 10%, about 0.0001% to about 5%, about 0.001% to about 5%, about 0.01% to about 5%, about 0.1% to about 5%, about 0.0001% to about 1%, about 0.001% to about 1%, about 0.01% to about 1%, about 0.1% to about 1%, about 0.5% to about 10%, or about 1% to 10% of Compound (21).

Embodiment 119. A composition comprising Compound (10); and:
(i) Compound (2), wherein the composition comprises about 0.0001% to about 10% of Compound (2) as measured by HPLC analysis, e.g., about 0.01% to about 10%, about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (2); and/or
(ii) Compound (4), wherein the composition comprises about 0.001% to about 10% of Compound (4) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (4); and/or
(iii) Compound (11A) or Compound (11B), wherein the composition comprises about 0.001% to about 10% of Compound (11A) or Compound (11B) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (11A) or Compound (11B); and/or
(iv) Compound (20A) or Compound (20B), wherein the composition comprises about 0.001% to about 10% of Compound (20A) or Compound (20B) as measured by HPLC analysis, e.g., about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (20A) or Compound (20B); and/or
(v) Compound (21), wherein the composition comprises about 0.0001% to about 10% of Compound (21) as measured by HPLC analysis, e.g., about 0.01% to about 10%, about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (21).

Embodiment 120. A composition comprising Compound (10) and Compound (2), wherein the composition comprises about 0.0001% to about 10% of Compound (2) as measured by HPLC analysis, e.g., about 0.01% to about 10%, about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (2).

Embodiment 121. A composition comprising Compound (10) and Compound (21), wherein the composition comprises about 0.0001% to about 10% of Compound (21) as measured by HPLC analysis, e.g., about 0.01% to about 10%, about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (21).

Embodiment 122. A composition comprising Compound (10); and:
(i) Compound (2), wherein the composition comprises about 0.0001% to about 10% of Compound (2) as measured by HPLC analysis, e.g., about 0.01% to about 10%, about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (2); and
(ii) Compound (21), wherein the composition comprises about 0.0001% to about 10% of Compound (21) as measured by HPLC analysis, e.g., about 0.01% to about 10%, about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (21).

Embodiment 123. A composition comprising Compound (10) and Compound (2), wherein the composition comprises about 85% to about 99.5% of Compound (10) as measured by HPLC analysis, e.g., about 90% to about 99.5%, or about 95% to about 99% of Compound (10), and about 0.0001% to about 10% of Compound (2) as measured by HPLC analysis, e.g., about 0.01% to about 10%, about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (2), and about 85% to about 99.5% of Compound (10), e.g., about 90% to about 99.5%, or about 95% to about 99% of Compound (10).

Embodiment 124. A composition comprising Compound (10) and Compound (21), wherein the composition comprises about 85% to about 99.5% of Compound (10) as measured by HPLC analysis, e.g., about 90% to about 99.5%, or about 95% to about 99% of Compound (10), and about 0.0001% to about 10% of Compound (21) as measured by HPLC analysis, e.g., about 0.01% to about 10%, about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (21).

Embodiment 125. A composition comprising Compound (10), Compound (2), and Compound (21), wherein the composition comprises about 85% to about 99.5% of Compound (10) as measured by HPLC analysis, e.g., about 90% to about 99.5%, or about 95% to about 99% of Compound (10), about 0.0001% to about 10% of Compound (2) as measured by HPLC analysis, e.g., about 0.01% to about 10%, about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (2), and about 0.0001% to about 10% of Compound (21) as measured by HPLC analysis, e.g., about 0.01% to about 10%, about 0.01% to about 10%, about 0.1% to about 10%, about 0.001% to about 5%, about 0.01% to about 5%, about 0.1% to about 5%, about 0.001% to about 1%, about 0.01% to about 1%, about 0.01% to about 0.5%, about 0.05% to about 0.5%, or about 0.1% to 0.5% of Compound (21).

Embodiment 126. Compound (21).

Embodiment 127. A composition comprising the compound of Embodiment 126.

Embodiment 128. Compound (22).

Embodiment 129 A composition comprising the compound of Embodiment 128.

Embodiment 130. A composition comprising the compound of Embodiments 1 or 2, and Compound (22), wherein the composition comprises about 0.0001% to about 10% of Compound (22) as measured by HPLC analysis, e.g., about 0.001% to about 10%, 0.01% to about 10%, about 0.1% to about 10%, about 0.0001% to about 5%, about 0.001% to about 5%, about 0.01% to about 5%, about 0.1% to about 5%, about 0.0001% to about 1%, about 0.001% to about 1%, about 0.01% to about 1%, about 0.1% to about 1%, about 0.5% to about 10%, or about 1% to 10% of Compound (22).

Embodiment 131. Compound (23).

Embodiment 132. Compound (24).

Embodiment 133. A composition comprising the compound of Embodiments 1 or 2, and Compound (23), wherein the composition comprises about 0.0001% to about 10% of Compound (23) as measured by HPLC analysis, e.g., about 0.001% to about 10%, 0.01% to about 10%, about 0.1% to about 10%, about 0.0001% to about 5%, about 0.001% to about 5%, about 0.01% to about 5%, about 0.1% to about 5%, about 0.0001% to about 1%, about 0.001% to about 1%, about 0.01% to about 1%, about 0.1% to about 1%, about 0.5% to about 10%, or about 1% to 10% of Compound (23).

Embodiment 134. A composition comprising the compound of Embodiments 1 or 2, and Compound (24), wherein the composition comprises about 0.0001% to about 10% of Compound (24) as measured by HPLC analysis, e.g., about 0.001% to about 10%, 0.01% to about 10%, about 0.1% to about 10%, about 0.0001% to about 5%, about 0.001% to about 5%, about 0.01% to about 5%, about 0.1% to about 5%, about 0.0001% to about 1%, about 0.001% to about 1%, about 0.01% to about 1%, about 0.1% to about 1%, about 0.5% to about 10%, or about 1% to 10% of Compound (24).

Embodiment 135. A composition comprising the compound of Embodiments 1 or 2, and Compound (25), wherein the composition comprises about 0.0001% to about 10% of Compound (25) as measured by HPLC analysis, e.g., about 0.001% to about 10%, 0.01% to about 10%, about 0.1% to about 10%, about 0.0001% to about 5%, about 0.001% to about 5%, about 0.01% to about 5%, about 0.1% to about 5%, about 0.0001% to about 1%, about 0.001% to about 1%, about 0.01% to about 1%, about 0.1% to about 1%, about 0.5% to about 10%, or about 1% to 10% of Compound (25).

Embodiment 136. A composition comprising the compound of Embodiments 1 or 2, and Compound (37), wherein the composition comprises about 0.0001% to about 10% of Compound (37) as measured by HPLC analysis, e.g., about 0.001% to about 10%, 0.01% to about 10%, about 0.1% to about 10%, about 0.0001% to about 5%, about 0.001% to about 5%, about 0.01% to about 5%, about 0.1% to about 5%, about 0.0001% to about 1%, about 0.001% to about 1%, about 0.01% to about 1%, about 0.1% to about 1%, about 0.5% to about 10%, or about 1% to 10% of Compound (37).

The present disclosure provides the following particular embodiments. The compound structures are provided in Table 1.

Embodiment #1. A compound having Formula (I):

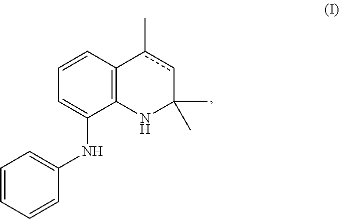

or a salt or solvate thereof, wherein [symbol] represents a single or double bond.

Embodiment 2. The compound of Embodiment #1, wherein the compound having Formula (I) is Compound (1).

Embodiment #3. The compound of Embodiment #1, wherein the compound having Formula (I) is Compound (2).

Embodiment #4. A composition comprising the compound of any one of Embodiments ##1-3, or a salt or solvate thereof.

Embodiment #5. A compound having Formula (II):

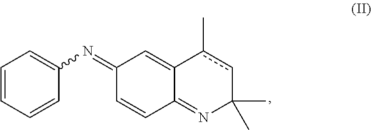

or a salt or solvate thereof, wherein [symbol] represents a single or double bond.

Embodiment #6. The compound of Embodiment #5, wherein the compound having Formula (II) is Compound (3).

Embodiment #7. The compound of Embodiment #5, wherein the compound having Formula (II) is Compound (4).

Embodiment #8. A composition comprising the compound of any one of Embodiments ##5-7, or a salt or solvate thereof.

Embodiment #9. A compound having Formula (III):

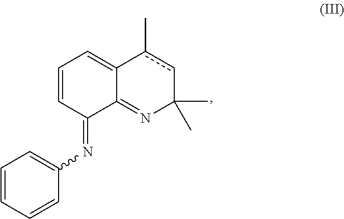

or a salt or solvate thereof, wherein [symbol] represents a single or double bond.

Embodiment #10. The compound of Embodiment #9, wherein the compound having Formula (III) is Compound (5).

Embodiment #11. The compound of Embodiment #9, wherein the compound having Formula (III) is Compound (6).

Embodiment #12. A composition comprising the compound of any one of Embodiments ##9-11, or a salt or solvate thereof.

Embodiment #13. A compound that is Compound (21), or a salt or solvate thereof.

Embodiment #14. A composition comprising the compound of Embodiment #13, or a salt or solvate thereof.

Embodiment #15. A compound that is Compound (23), or a salt or solvate thereof.

Embodiment #16. A composition comprising the compound of Embodiment #15, or a salt or solvate thereof.

Embodiment #17. A compound that is Compound (24), or a salt or solvate thereof.

Embodiment #18. A composition comprising the compound of Embodiment #17, or a salt or solvate thereof.

Embodiment #19. A composition comprising a compound having Formula (IV):

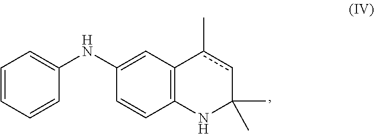

or a salt or solvate thereof, and:
(a) a compound having Formula (I):

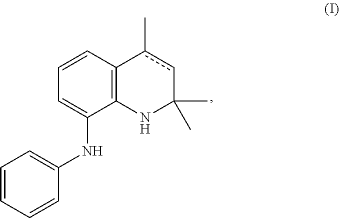

or a salt or solvate thereof; or:
(b) a compound having Formula (II):

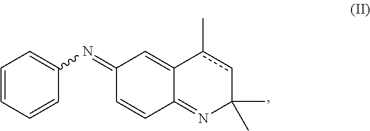

or a salt or solvate thereof; or
(c) a compound having Formula (III):

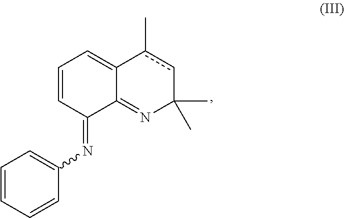

or a salt or solvate thereof; or
(d) Compound (7), or a salt or solvate thereof, or
(e) Compound (8), or a salt or solvate thereof, or
(f) Compound (11A) or Compound (11B), or a salt or solvate thereof, or
(g) Compound (20A) or Compound (20B), or a salt or solvate thereof, or
(h) Compound 21, or a salt or solvate thereof, or
(i) Compound (22), or a salt or solvate thereof, or
(j) Compound (23), or a salt or solvate thereof, or
(k) Compound (24), or a salt or solvate thereof; or
(l) a combination of Formula (I), or a salt or solvate thereof; Formula (II), or a salt or solvate thereof; Formula (III), or a salt or solvate thereof, Compound (7), or a salt or solvate thereof, Compound (8), or a salt or solvate thereof, Compound (11A) or Compound (11B), or a salt or solvate thereof, Compound (20A) or Compound (20B), or a salt or solvate thereof, and/or Compound (21), or a salt or solvate thereof, Compound (22), or a salt or solvate thereof, Compound (23), or a salt or solvate thereof, and/or Compound (24), or a salt or solvate thereof,
wherein [symbol] represents a single or double bond.

Embodiment #20. The composition of Embodiment #19 comprising a compound having Formula (IV), or a salt or solvate thereof, and a compound having Formula (I), or a salt or solvate thereof

Embodiment #21. The composition of Embodiments ##19 or 20, comprising about 0.1 to about 10% of Formula (I) as measured by high performance liquid chromatography (HPLC) analysis.

Embodiment #22. The composition of Embodiments ##19 or 20, wherein the weight ratio of Formula (I) to Formula (IV) is 0.001 to 0.1.

Embodiment #23. The composition of any one of Embodiments ##19-33 comprising a compound having Formula (IV), or a salt or solvate thereof, and a compound having Formula (II), or a salt or solvate thereof.

Embodiment #24. The composition of any one of Embodiments #19-23, comprising about 0.1 to about 10% of Formula (II) as measured by HPLC analysis.

Embodiment #25. The composition of any one of Embodiments #19-23, wherein the weight ratio of Formula (II) to Formula (IV) is 0.001 to 0.1.

Embodiment #26. The composition of any one of Embodiments #19-25 comprising a compound having Formula (IV), or a salt or solvate thereof, and a compound having Formula (III), or a salt or solvate thereof.

Embodiment #27. The composition of any one of Embodiments #19-26, comprising about 0.1 to about 10% of Formula (III) as measured by HPLC analysis.

Embodiment #28. The composition of any one of Embodiments #19-26, wherein the weight ratio of Formula (IIII) to Formula (IV) is 0.001 to 0.1.

Embodiment #29. The composition of any one of Embodiments #19-28 comprising a compound having Formula (IV), or a salt or solvate thereof, and Compound (7), or a salt or solvate thereof.

Embodiment #30. The composition of any one of Embodiments #19-29, comprising about 0.1 to about 10% of Compound (7) as measured by HPLC analysis.

Embodiment #31. The composition of any one of Embodiments #19-29, wherein the weight ratio of Compound (7) to Formula (IV) is 0.001 to 0.1.

Embodiment #32. The composition of any one of Embodiments #19-31, comprising a compound having Formula (IV), or a salt or solvate thereof, and Compound (11), or a salt or solvate thereof.

Embodiment #33. The composition of any one of Embodiments #19-32, comprising about 0.001 to about 10% of Compound (8) as measured HPLC analysis.

Embodiment #34. The composition of any one of Embodiments #19-32, wherein the weight ratio of Compound (8) to Formula (IV) is 0.001 to 0.1.

Embodiment #35. The composition of any one of Embodiments #19-34, comprising about 0.001 to about 10% of Compound (11A) or Compound (11B) as measured by HPLC analysis.

Embodiment #36. The composition of any one of Embodiments #19-34, wherein the weight ratio of Compound (11A) or Compound (111B) to Formula (IV) is 0.001 to 0.1.

Embodiment #37. The composition of any one of Embodiments #19-36, comprising a compound having Formula (IV), or a salt or solvate thereof, and Compound (21), or a salt or solvate thereof.

Embodiment #38. The composition of any one of Embodiments #19-37, comprising about 0.1 to about 10% of Compound (21) as measured by HPLC analysis.

Embodiment #39. The composition of any one of Embodiments #19-37, wherein the weight ratio of Compound (21) to Formula (IV) is 0.001 to 0.1.

Embodiment #40. The composition of any one of Embodiments #19-39, comprising a compound having Formula (IV), or a salt or solvate thereof, and Compound (23), or a salt or solvate thereof.

Embodiment #41. The composition of any one of Embodiments #19-40, comprising about 0.1 to about 10% of Compound (23) as measured by HPLC analysis.

Embodiment #42. The composition of any one of Embodiments #19-40, wherein the weight ratio of Compound (23) to Formula (IV) is 0.001 to 0.1.

Embodiment #43. The composition of any one of Embodiments #19-42, comprising a compound having Formula (IV), or a salt or solvate thereof, and Compound (24), or a salt or solvate thereof.

Embodiment #44. The composition of any one of Embodiments #19-43, comprising about 0.01 to about 10% of Compound (23) as measured by HPLC analysis.

Embodiment #45. The composition of any one of Embodiments #19-43, wherein the weight ratio of Compound (23) to Formula (IV) is 0.0001 to 0.1.

Embodiment #46. The composition of any one of Embodiments #19-45, wherein the compound having Formula (IV) is Compound (9).

Embodiment #47. The composition of any one of Embodiments #19-47, wherein the compound having Formula (IV) is Compound (10).

Embodiment #48. A composition comprising Compound (10); and
(i) about 0.001% to about 10% of Compound (2) as measured by HPLC analysis; and/or
(ii) about 0.001% to about 10% of Compound (4) as measured by HPLC analysis, and/or
(iii) about 0.001% to about 10% of Compound (11A) or Compound (11B) as measured by HPLC analysis, and/or
(iv) about 0.001% to about 20% of Compound (20A) or Compound (20B), as measured by HPLC analysis, and/or
(v) about 0.001% to about 10% of Compound (21) as measured by HPLC analysis, and/or
(vi) about 0.0001 to about 3% of Compound (23) as measured by HPLC analysis, and/or
(vii) about 0.0001 to about 3% of Compound (24) as measured by HPLC analysis.

Embodiment #49. The composition of Embodiment #48 comprising Compound (10) and about 0.001% to about 10% of Compound (2) as measured by HPLC analysis.

Embodiment #50. The composition of Embodiments #48 or 49 comprising Compound (10) and about 0.01% to about 1% of Compound (2) as measured by HPLC analysis.

Embodiment #51. The composition of Embodiments #8 or 49 comprising Compound (10) and about 0.03% to about 3% of Compound (2) as measured by HPLC analysis.

Embodiment #52. The composition of Embodiment 8 comprising Compound (10) and about 0.001% to about 10% of Compound (21) as measured by HPLC analysis.

Embodiment #53. The composition of any one of Embodiments #48-52 comprising Compound (10) and about 0.01% to about 1% of Compound (21) as measured by HPLC analysis.

Embodiment #54. The composition of any one of Embodiments #48-52 comprising Compound (10) and about 0.03% to about 3% of Compound (21) as measured by HPLC analysis.

Embodiment #55. The composition of any one of Embodiments #48-52 comprising about 0.02% to about 2% of Compound (11A) or Compound (11B) as measured by HPLC analysis.

Embodiment #56. The composition of any one of Embodiments #48-52 comprising about 1% to about 20% of Compound (20A) or Compound (20B) as measured by HPLC analysis.

Embodiment #57. The composition of any one of Embodiment #s 48-56 comprising about 90% to about 99% of Compound (10) as measured by HPLC analysis.

Embodiment #58. The composition of any one of Embodiments #4, 8, 12, 14, 16, 18-57 further comprising:
(i) one or more elastomers; or
(ii) one or more fillers; or
(iii) one or more rubber chemicals; or
(iv) one or more plasticizers; or
(v) one or more second antidegradants; or
(vi) a combination of one or more elastomers, one or more fillers, one or more rubber chemicals, one or more plasticizers, and/or one or more second antidegradants.

Embodiment #59. A vulcanized elastomeric article comprising the composition of Embodiment #58.

Embodiment #60. A vehicle comprising the vulcanized elastomeric article of Embodiment #59.

Embodiment #61. A kit comprising the composition of Embodiment #58 and instructions for using the composition to prepare a vulcanized elastomeric article.

Embodiment #62. A process for preparing the composition of any one of Embodiments #4, 8, 14, 16, 18-45, the process comprising reacting a composition comprising 4-aminodiphenylamine with acetone, 2,2-dimethoxypropane, or mesityl oxide in the presence of a strong acid acid.

Embodiment #63. A composition comprising the compound of Embodiments #2,3,6,7, 10, 11, 13, 15, and 17 and:
(i) one or more elastomers; or
(ii) one or more fillers; or
(iii) one or more rubber chemicals; or
(iv) one or more plasticizers; or
(v) one or more second antidegradants; or
(vi) a combination of one or more elastomers, one or more fillers, one or more rubber chemicals, one or more plasticizers, and/or one or more second antidegradants.

Embodiment #64. A vulcanized elastomeric article comprising the composition of Embodiment #63.

Embodiment #65. A vehicle comprising the vulcanized elastomeric article of Embodiment #64.

Embodiment #66. A kit comprising the composition of Embodiment #65 and instructions for using the composition to prepare a vulcanized elastomeric article.

### EXAMPLES

### Example 1

Reaction of 4-Aminodiphenylamine with Acetone

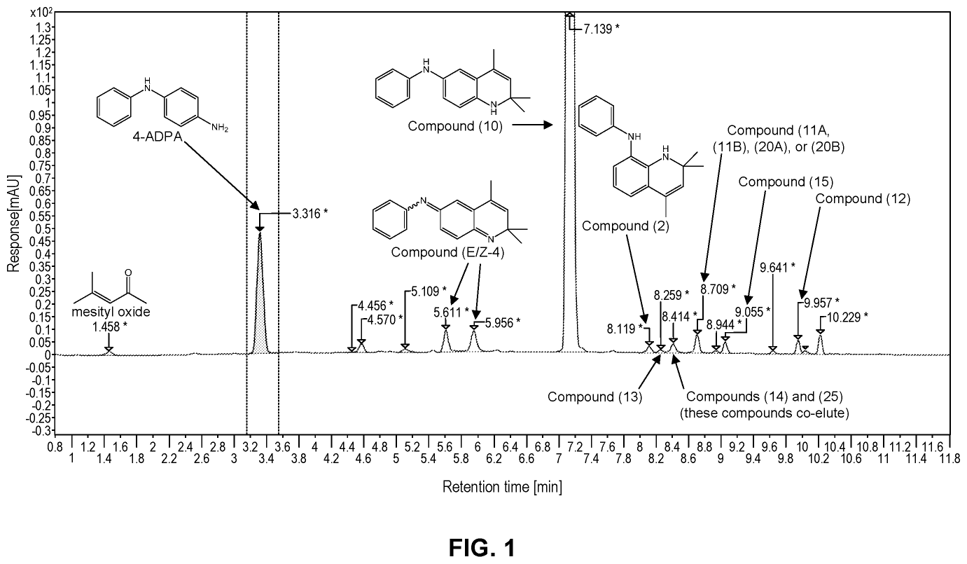

Charged 184.0 g of 4-aminodiphenylamine (1.0 moles; 99.2% purity) and 8.3 mL concentrated hydrochloric acid (0.1 moles, 37%, 6=1.18) catalyst to 500-mL kettle-style reactor equipped with mechanical stirrer, temperature control, addition funnel, nitrogen inlet and condenser with receiver. Heated to 95° C. under nitrogen with stirring. Fed 440 mL acetone (6.0 moles, technical grade, 6=0.784) from addition funnel to stirred reactor over 4.5 hours at 95° C. under nitrogen. Transferred 265 mL acetone collected in receiver to addition funnel and feed to reactor over 1.5 hours at 95° C. with stirring under nitrogen. Neutralized reaction mass with 60 mL 15% aqueous sodium carbonate. Cooled to room temperature. Transferred contents of reactor to separatory funnel with 200 mL toluene and 50 mL water in 2 portions. Separated layers and discarded aqueous layer. Removed toluene by rotary evaporation. Recovered 212 g of 37% 6-anilino-2,2,4-trimethyl-1,2-dihydroquinoline, i.e., Compound (10), and 54% unreacted 4aminodiphenylamine (“4-ADPA”) starting material by area percent HPLC. Separated 4ADPA from Compound (10) by preparing a crude solution of approx. 20 g of reaction mixture in 30 mL of chloroform, loading onto a sintered glass filter (13.3 cm diameter, 2000-mL size) prepared with 350-400 g of silica gel 60, eluting with 3 to 4 L of additional chloroform, collecting the clean filtrates (spot checked for effectiveness of 4-ADPA separation by thin layer chromatography) in 500-mL portions, and removing volatiles via rotary evaporation. Analysis of final material purified by silica column filter showed 95.3% of Compound (10) and other unexpected molecules, i.e., byproducts, as measured by HPLC. See FIG. 1, Table 1, and Table 2. It has not yet been determined whether the HPLC peak at 8.7 min is Compound (11 A), Compound (11B), Compound (20A), or Compound (20B).

TABLE 2

| Retention Time | | HPLC % | Mass Charge |
|---|---|---|---|
| (min) | Compound | area | Ratio |
| 5.6; 6.0 | E/Z-(4) | 0.36; 0.39 | 263 |
| 7.1 | (10) | 95.3 | 265 |
| 8.1 | (2) | 0.10 | 265 |
| 8.3 | (13) | 0.05 | 489 |
| 8.4 | (14), (25) | 0.16 | 445, 279 |
| 8.7 | (11A), (11B), (20A), | 0.25 | 305 |
| | or (20B) | | |
| 9.1 | (15) | 0.15 | 445 |
| 10.0 | (12) | 0.16 | 529 |

### Method 1. HPLC Analysis

Chromatograms were obtained using an Agilent 1260 Infinity II series HPLC system equipped with a Poroshell 120 column (EC-C18 4.6×50 mm, 2.7 tm) kept at 25° C. with 5 μL injection volume. A diode array detector was used to measure the UV absorption of the effluent at multiple wavelengths in the 200 to 450 nm range (2 nm step). The wavelength 260 nm was used for determination of each component percentage by peak area. Analytes can be reliably detected at or above 0.5 ppm. The molecular weights of each peak observed in the chromatograms were identified by MS coupled to the LC-UV system using a single quadrupole mass selective detector with atmospheric-pressure chemical ionization in positive mode. The source parameters were set as follows: The temperatures of the gas and vaporizer were 300° C. and 200° C. respectively, the gas flow was 5.0 L/min, and the nebulizer pressure was 15 PSI. The mobile phase (1 mL/min flow rate) consisted of 5 mmol/L aqueous ammonium formate (Solvent A) and acetonitrile (Solvent B) using the following gradient shown in Table 3.

TABLE 3

| Time (min) | Solvent A (%) | Solvent B (%) |
|---|---|---|
| 0.00 | 70 | 30 |
| 0.50 | 70 | 30 |
| 9.00 | 5 | 95 |
| 16.00 | 5 | 95 |
| 18.00 | 70 | 30 |
| 20.00 | 70 | 30 |

### Method 2: HPLC-UV/MS

Chromatograms were obtained using an Agilent 1260 Infinity II series HPLC system equipped with an Agilent Poroshell 120 column (EC-C18 4.6×100 mm, 2.7 m, PN:695975-902) kept at 25° C. with 5 μL injection volume. A diode array detector was used to measure the UV absorption of the effluent at multiple wavelengths in the 200 to 450 nm range (2 nm step). The wavelength 290 nm (bw=20) could be used for detection of each component percentage by peak area. The molecular weights of each peak observed in the chromatograms were identified by MS coupled to the LC-UV system using an Agilent InfinityLab LC/MSD G6125C single quadrupole mass selective detector in APCI+mode with a fragmentation voltage of 100 V. Compounds were detected as parent ion protonated form [M+1]⁺. The source parameters were set as follows: The temperatures of the gas and vaporizer were 300° C. and 200° C. respectively, the gas flow was 5.0 L/min, and the nebulizer pressure was 15 PSI. For selected ion monitoring (SIM) detection, the mass spectrometer was set to monitor the intensity of response of the selected compound at m/z=[M+1]⁺. For SIM detection of Compounds (2), (21), (23), and (25) the mass spectrometer was set to m/z=265, 225, and 279 amu, respectively. The mobile phase (1 mL/min flow rate) consisted of 75 mM aqueous formic acid and 5 mM aqueous ammonium formate buffer solution (Solvent D) and acetonitrile (Solvent B) using the following gradient shown in Table 4.

TABLE 4

| Time (min) | Solvent B (%) | Solvent D (%) |
|---|---|---|
| 0.00 | 20 | 80 |
| 1.00 | 20 | 80 |
| 29.00 | 95 | 5 |
| 38.00 | 95 | 5 |
| 40.00 | 20 | 80 |
| 42.00 | 20 | 80 |

To minimize interference from high concentrations of Compound (10), when using SIM a diverter valve was used to divert the eluent from the MS detector to waste during the elution time of Compound (10) during the acquisition.

To determine the experimental limit of detection LOD or lower limit of the working calibration range, two stock solutions of Compound (21) were prepared with concentrations of 470 ppm (g/ml) and 958 ppm (g/ml). A 10:1 series dilution was applied to each stock solution until concentrations of 4.70 parts per billion (ppb) and 0.958 ppb were achieved respectively. Standards with concentration between 4.70 ppm and 0.958 ppb (eight total standards) were injected using Method 2 and a linear calibration with offset (origin ignored) was used to evaluate the calibration range. The lower limit of the working range was determined by the lowest concentration standard with a relative residual percent within +/−20%. For Molecule (21) the LOD was determined to be 9.58 parts per billion having a residual of −19.3%.

### Method 3: GC-MS

Chromatograms were obtained using a Thermo Fisher Trace 1310 Gas Chromatograph with an ISQ7000 single quadrupole mass spectrometer for detection. One microliter of sample solution was injected with an AI 1310 autosampler into a split inlet with a split flow of 15 ml/min and purge flow of 5 ml/min while maintaining an inlet temperature of 250° C. Helium was used as the carrier gas at a constant flow rate of 1 ml/min with an Agilent VF-5 ms capillary column (15 m×0.25 mm×1 m) used for the stationary phase. The initial oven temperature of 35° C. was held for one minute before ramping at 15° C./min up to 240° C. The oven was held at 240° C. for 3 minutes followed by an 8° C./min ramp up to 300° C. where it was held isothermal for 20 minutes giving a total method time of 45.2 minutes. The mass spectrometer transfer line and ion source temperatures were both 300° C. Electron impact (EI) ionization mode was utilized to collect total ion chromatograms from 40-550 amu (0.1s dwell time) for general identification while targeted mass ranges with the same ionization mode and parameters were used for quantitative analysis of specific analytes of interest. Target mass ranges depended on the analyte and were selected from the most abundant ion in spectra generated from the above conditions. Ion ranges selected were 208-210 amu for Compound (21), 262-265 amu for Compounds (23) and (25), and 248-250 amu for Compound (2). The methods were calibrated with purified external standards diluted to multi levels typically between 50 ppb and 5000 ppb though ranges may vary depending on the concentration of the observed analyte. Chromeleon CDS was used for spectra and quantitative analysis.

To estimate the experimental limit of detection LOD or lower limit of the working calibration range, two stock solutions of Compound (25) were prepared with concentrations of 554 ppm (g/ml) and 1184 ppm (g/ml). A 10:1 series dilution was applied to each stock solution until concentrations of 5.54 parts per billion (ppb) and 1.18 ppb were achieved respectively. Standards with concentration between 5.54 ppm and 1.18 ppb (eight total standards) were injected using Method 3 and a linear calibration with offset (origin ignored) was used to evaluate the calibration range. The lower limit of the working range was estimated by the lowest concentration standard with a relative residual percent within +/−20%. For Molecule (25) the LOD was determined to be 55.4 parts per billion having a residual of −5.75%.

### Example 2

### Reaction of 4-Aminodiphenylamine with Mesityl Oxide

Charged 5.54 g 4-aminodiphenylamine (30 mmol; 99.2% purity) and 0.5 mL concentrated hydrochloric acid (6 mmol, 37%, 6=1.18) catalyst to 50-mL reactor equipped with magnetic stirrer, temperature control, addition funnel, nitrogen inlet, and reflux condenser with receiver. Heated to 100° C. under nitrogen with stirring. Fed 23 mL mesityl oxide (180 mmol, 90% technical grade, Sigma, 6=0.858) over 2 hours at 100° C. under nitrogen with stirring. Hold for another 2.5 hours under the same conditions, monitoring the reaction for completion by HPLC analysis (Method 1). Neutralized with 4 mL 15% aqueous sodium carbonate. Cooled to room temperature. Transferred contents of reactor to separatory funnel with 50 mL chloroform and 20 mL water in 2 portions. Separated layers and discarded aqueous layer. Removed chloroform and some portion of mesityl oxide by rotary evaporation. Recovered 9.7 g of 59.1% Compound (10), approximately 10% total unreacted starting materials (9% mesityl oxide and 1% 4-aminodiphenylamine), and other unexpected molecules, i.e., byproducts, as measured by area percent HPLC. See EXAMPLE 1 for details of HPLC analysis, Table 1, and Table 5. It has not yet been determined whether the HPLC peak at 7.3 min is Compound (11A), Compound (11B), Compound (20A), or Compound (20B).

TABLE 5

| Retention Time | | HPLC % | Mass Charge |
|---|---|---|---|
| (minutes) | Compound | area | Ratio |
| 5.6; 6.0 | E/Z-(4) | 0.25; 0.19 | 263 |
| 6.9 | (17) | 1.8 | 363 |
| 7.1 | (10) | 59.1 | 265 |
| 7.3 | (11A), (11B), (20A) | 9.9 | 305 |
| | or (20B) | | |
| 8.5 | (18) | 2.3 | 345 |
| 8.6 | (19) | 2.6 | 345 |

### Example 3

### Reaction of 4-Aminodiphenylamine with Acetone and Mesityl Oxide

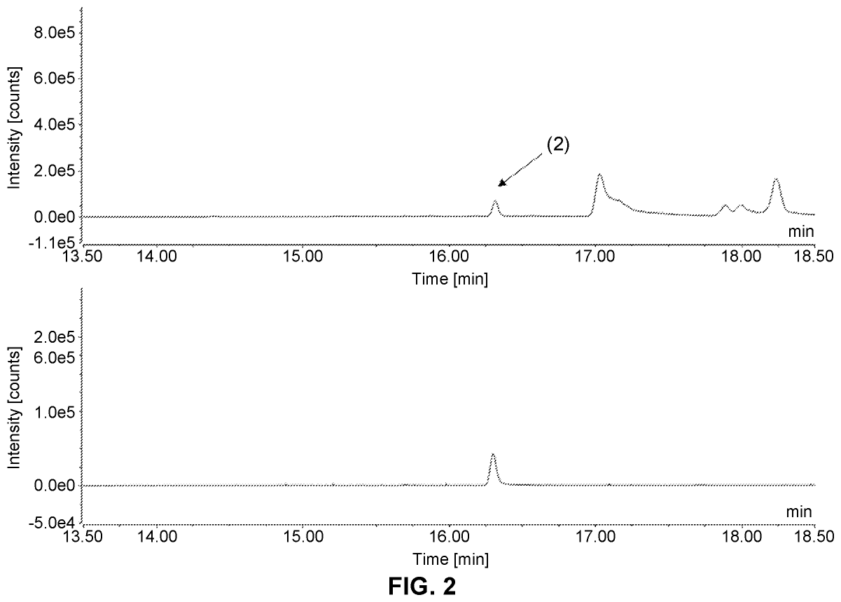

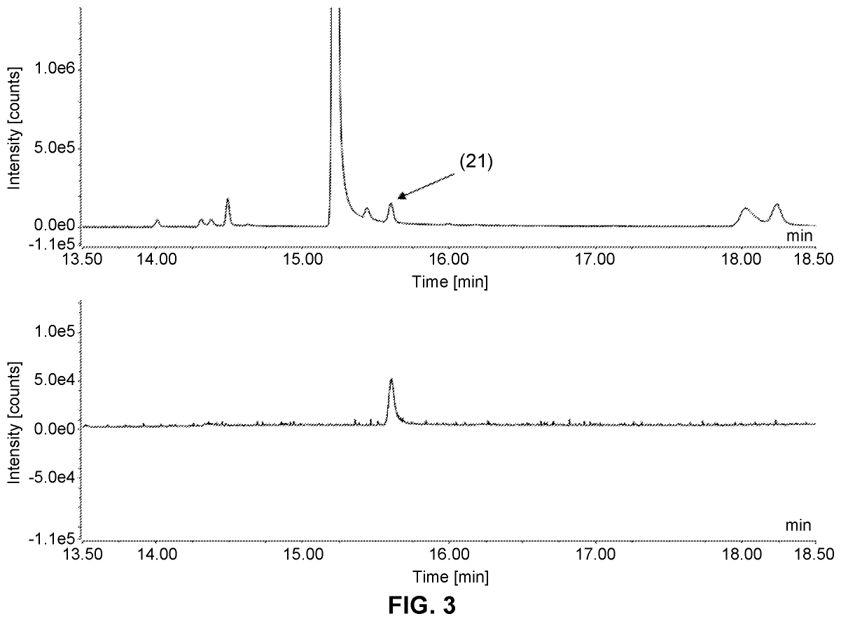

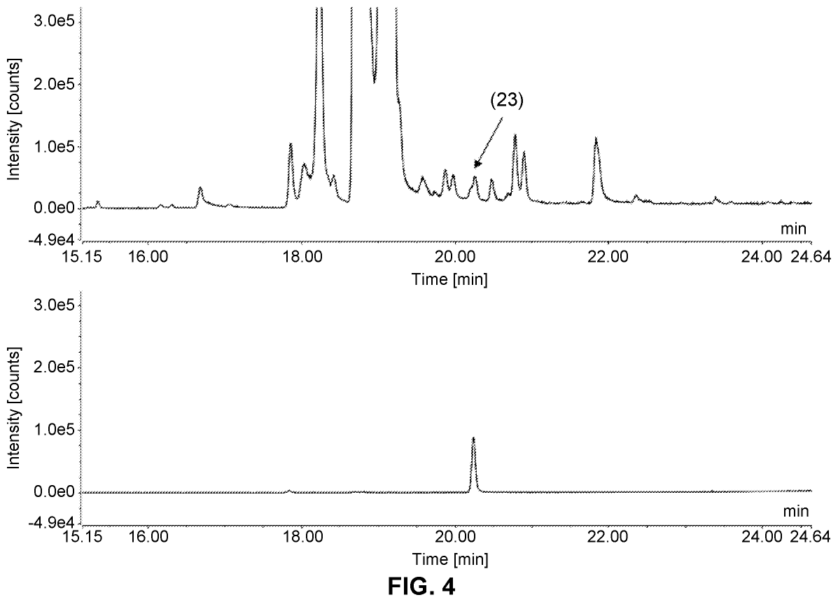

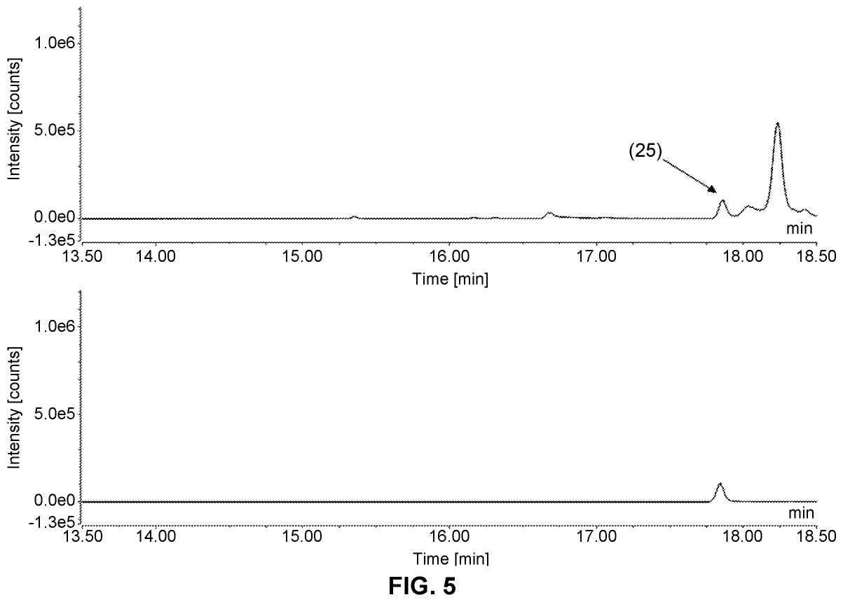

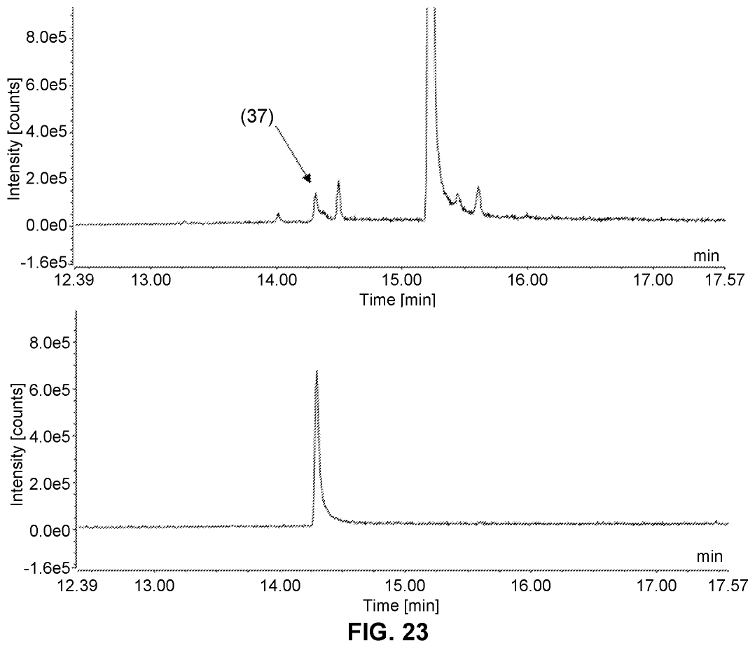

To a stirring solution of 4-Aminodiphenylamine (1.50 g, 8.14 mmol) in 3:2 v:v acetone:mesityl oxide (5 mL) at room temperature was added zinc triflate (0.296 g, 0.814 mmol). The stirring mixture was heated to 80° C. and maintained for 22 h. The mixture was then allowed to settle and cool for 10 minutes at room temperature. The solution was then decanted by pouring into a clean flask. The remaining sediment was rinsed with 20 mL of acetone in two portions which were combined with the solution in the flask. The resulting solution was then evaporated under reduced pressure to give 3.08 g of material containing Compound (10) and other unexpected molecules, i.e., byproducts, including Compounds (2), (21), (23), (25), and (37). FIGS. 2-5 and 23.

### Example 4

### Reaction of 4-Aminodiphenylamine with Acetone

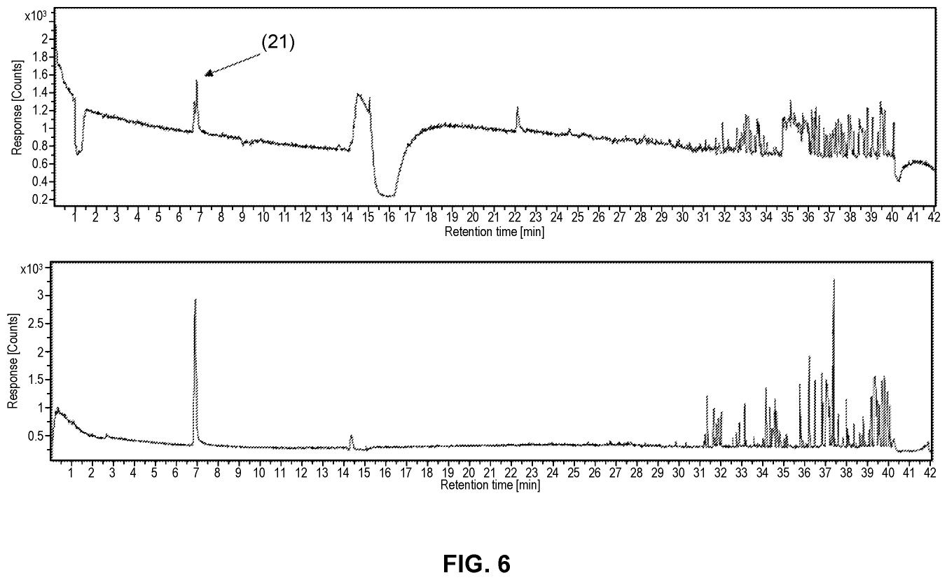

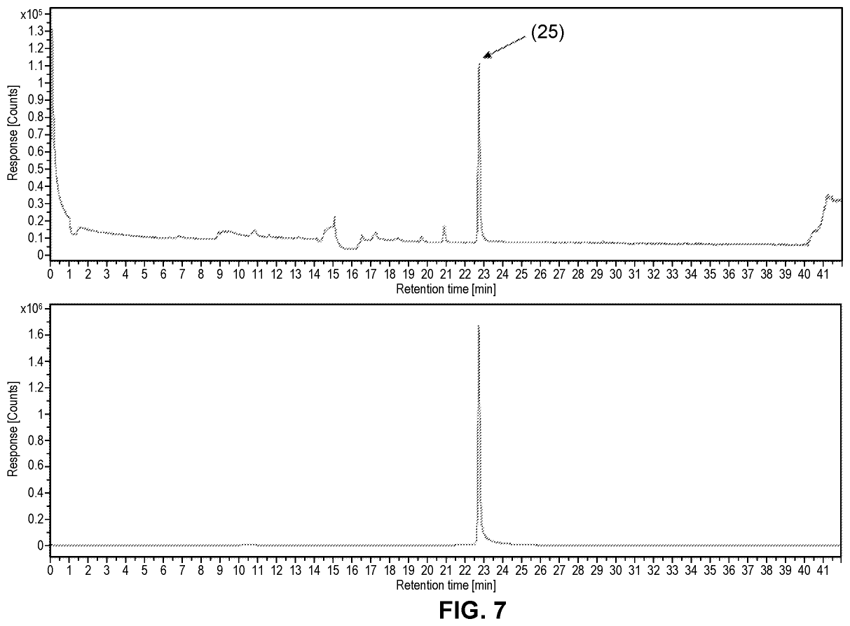

To a solution of 4-aminodiphenylamine (750.0 g, 2.7 mol) in acetone (1800 mL) was added zinc triflate (148 g, 0.27 mol), under nitrogen atmosphere at room temperature. The resulting mixture was heated to 60-65° C. and maintained for 20 h. After completion of the reaction, by TLC, cooled to room temperature and filtered through celite bed. The celite bed was washed with ethyl acetate, collected the filtrate and evaporated under reduced pressure. This procedure was repeated 4 times, and the crude materials were combined. The combined crude material was purified by column chromatography using silica gel (100-200 mesh) eluting with 5-10% ethyl acetate in hexane to afford 1534.0 g of purified Compound (10) as a light-yellow solid. Analysis of the final material by Method 1 showed 98.5% Compound (10) and other unexpected molecules, i.e., byproducts. FIGS. 6 and 7.

### Example 5

### Reaction of 2-Aminodiphenylamine with Acetone

Charged 5.53 g 2-aminodiphenylamine (30 mmol, Sigma) and 0.25 mL concentrated hydrochloric acid (3 mmol, 37%, δ=1.18) catalyst to 50-mL reactor equipped with magnetic stirrer, temperature control, addition funnel, nitrogen inlet, and reflux condenser with receiver. Heated to 95° C. under nitrogen with stirring. Fed 13.5 mL acetone (180 mmol, ACS grade, δ=0.784) over 1.5 hours at 95° C. under nitrogen with stirring. Held for another 1.5 hours under the same conditions. Neutralized with 2 mL 15% aqueous sodium carbonate. Cooled to room temperature. Transferred contents of reactor to separatory funnel with 50 mL toluene and 20 mL water in 2 portions. Separated layers and discarded aqueous. Removed toluene by rotary evaporation. Recovered 5.6 g of material comprising 4.5% of 8-anilino-2,2,4-trimethyl-1,2-dihydroquinoline (Compound (2), 65.6% unreacted 2-aminodiphenylamine (“2-ADPA”) starting material, and other molecules, i.e., byproducts, as measured by area percent HPLC. See EXAMPLE 1 for details of HPLC analysis and Table 6.

TABLE 6

| Retention Time | | HPLC % | Mass Charge |
|---|---|---|---|
| (minutes) | Compound | area | Ratio |
| 4.5 | 2-ADPA | 65.6 | 185 |
| 5.2 | (21) | 13.8 | 225 |
| 6.1 | (7) | 13.2 | 225 |
| 8.1 | (2) | 4.5 | 265 |

### Example 6

### Reaction of 2-Aminodiphenylamine with Mesityl Oxide

Charged 4.28 g 2-aminodiphenylamine (23.2 mmol, Sigma) and 0.2 mL concentrated hydrochloric acid (2.4 mmol, 37%, δ=1.18) catalyst to 50-mL reactor equipped with magnetic stirrer, temperature control, addition funnel, nitrogen inlet, and reflux condenser with receiver. Heated to 95° C. under nitrogen with stirring. Fed 11 mL mesityl oxide (91.4 mmol, 95%, TCI, δ=0.858) over 1 hour at 95° C. under nitrogen with stirring. Held for another 1.5 hours under the same conditions. Neutralized with 1.5 mL 15% aqueous sodium carbonate. Cool to room temperature. Analysis of crude reaction mass showed 4.6% of Compound (2), 65.1% total unreacted starting materials (42.3% mesityl oxide and 22.8% 2-ADPA), and other byproducts as measured by area percent HPLC. See EXAMPLE 1 for details of HPLC analysis and Table 7. It has not yet been determined whether the HPLC peak at 8.4 min is Compound (11A), Compound (11B), Compound (20A), or Compound (20B).

TABLE 7

| Retention Time | | HPLC % | Mass Charge |
|---|---|---|---|
| (minutes) | Compound | area | Ratio |
| 1.5 | Mesityl oxide | 42.3 | |
| 4.5 | 2-ADPA | 22.8 | 185 |
| 5.2 | (21) | 6.2 | 225 |
| 6.2 | (7) | 5.1 | 225 |
| 8.1 | (2) | 4.6 | 265 |
| 8.4 | (11A), (11B), (20A) | 6.4 | 305 |
| | or (20B) | | |

### Example 7

### Synthesis of Compound (4) (2,2,4-trimethyl-N-phenylquinolin-6(2H)-imine)

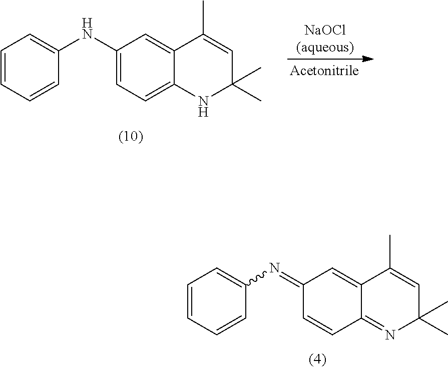 [SMILES: CC1=CC(C)(C)Nc2ccc(Nc3ccccc3)cc21 ; CC1=CC(C)(C)N=C2C=CC(=Nc3ccccc3)C=C12]

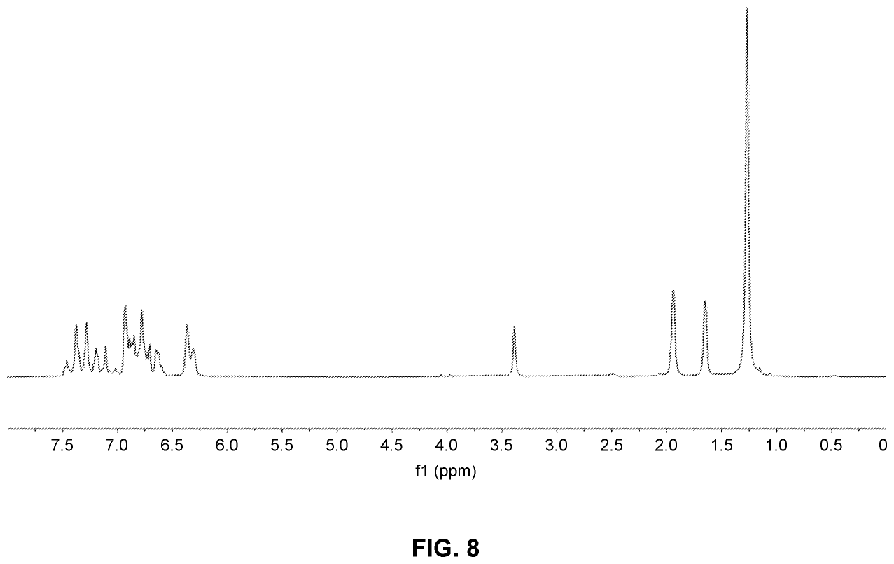

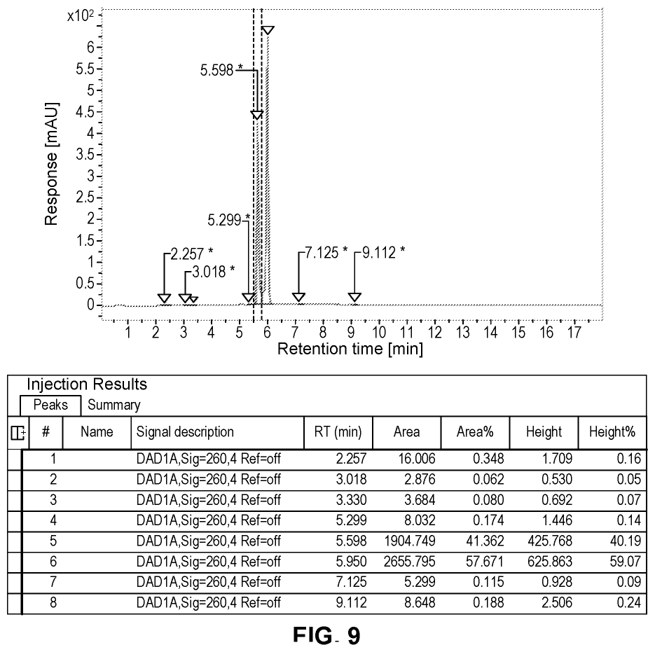

A 250-mL round bottom flask fitted with a stir-bar was loaded with 2,2,4-trimethyl-N-phenyl-1,2-dihydroquinolin-6-amine (5.5 g; 20.8 mmol), then blanketed with N2. Acetonitrile (60 mL) was added and the mixture was stirred (the substrate dissolved within minutes). Aqueous 10-15% NaOCl (16 mL; 25.9-38.9 mmol hypochlorite anion) was portion-wise added over 6-7 min. A dense liquid separated immediately. The 2-phase system was stirred without external cooling or heating. A gradual and moderate exotherm ensued, peaking at about 25 min. After 30 min of reaction, silica gel TLC using 20% AcOEt in hexanes as the eluent showed complete consumption of the substrate (Rf=0.45), as well as formation of 2 new dots (Rf=0.2 and 0.4). After about 50 minutes of reaction, aqueous 1.69 mol/L Na₂SO₃ (15 mL; 25.35 mmol sulfite anion) was added to quench excess hypochlorite anion (an exotherm was observed). The 2-phase system was diluted with AcOEt (60 mL) in a separatory funnel. The aqueous phase was discarded. The organic phase was washed with deionized water (60 mL), with aq. diluted NaCl (60 mL), with aq. saturated NaCl (60 mL), dried over anhydrous MgSO₄, filtered, then stripped of volatiles under reduced pressure (rotary evaporator; water bath=50° C.). Further drying on the rotary evaporator (50° C.; approx. 10 mbars) until constant weight afforded a dark viscous liquid (4.7 g; 86% yield with respect to 2,2,4-trimethyl-N-phenyl-1,2-dihydroquinolin-6-amine). The structure was authenticated by NMR spectroscopy, as well as LC-MS m/z [M+1]⁺=263. The purity in Compound (4) was 99.03% (combined areas of the 2 stereoisomers) as measured by LC-UV 260 nm. The proton NMR spectrum (DMSO-d₆) and HPLC chromatogram are depicted in FIG. 8 and FIG. 9, respectively.

### Example 8

### Synthesis of Compound (2) (2,2,4-trimethyl-N-phenyl-1,2-dihydroquinolin-8-amine) and Compound (21) (11,11-dimethyl-10,11-dihydro-5H-dibenzo[b,e][1,4]diazepine)

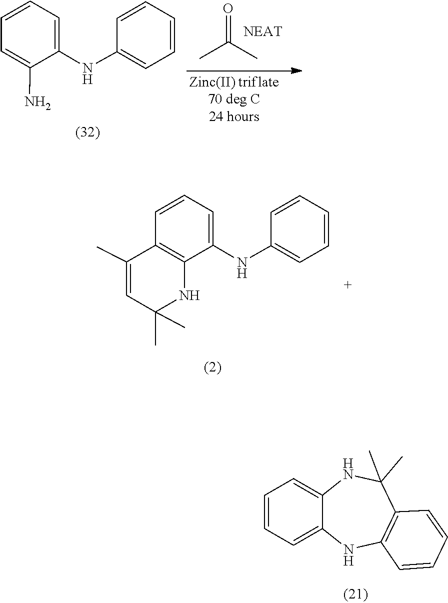

To a 100-mL round bottom flask fitted with a stir-bar and a reflux condenser was charged Compound (32) (10.48 g; 56.9 mmol) and acetone (17.0 mL; 232 mmol). The mixture was stirred under N2 protection (2-ADPA dissolved readily). Zinc triflate (2.15 g; 5.91 mmol) was added in one portion and the reaction mixture was gradually heated with an oil bath (set temperature=70 deg C.). Once the temperature was attained, it was maintained for 24 hours. Upon cooling to room temperature (overnight), The viscous residue was diluted with a mixture of toluene (2 mL) and acetone (3 mL). The obtained free flowing dark liquid (containing traces of insoluble materials) was loaded into a 41 mm inner diameter column prepared with silica gel 60 and 10% AcOEt in hexanes. The height of wet silica gel in the column was 25-26 cm. The flask and column walls were rinsed with toluene (1 mL). The column was eluted with 10% AcOEt in hexanes. Each portion coming out was analyzed by silica gel TLC. Fraction 1 containing the material having a Rf of 0.5 (10% AcOEt in hexanes) was collected. The column was eluted with 30% AcOEt in hexanes. Fraction 2 containing the material having a Rf of 0.2 (10% AcOEt in hexanes) was collected. Each fraction was independent stripped of volatiles under reduced pressure (rotary evaporator; water bath=55 deg C.). Further drying on the rotary evaporator (60 deg C.; 5-15 mbars) until constant weight afforded the two products.

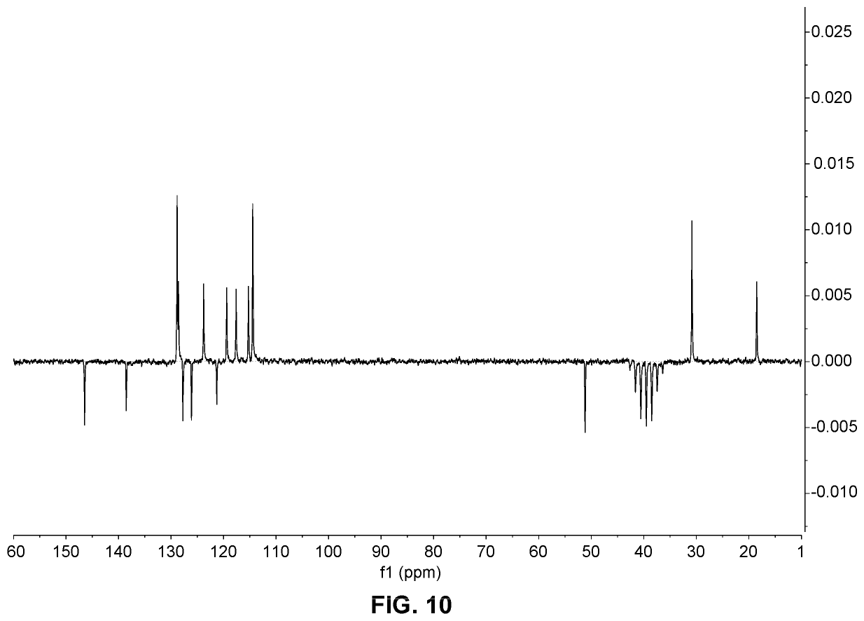

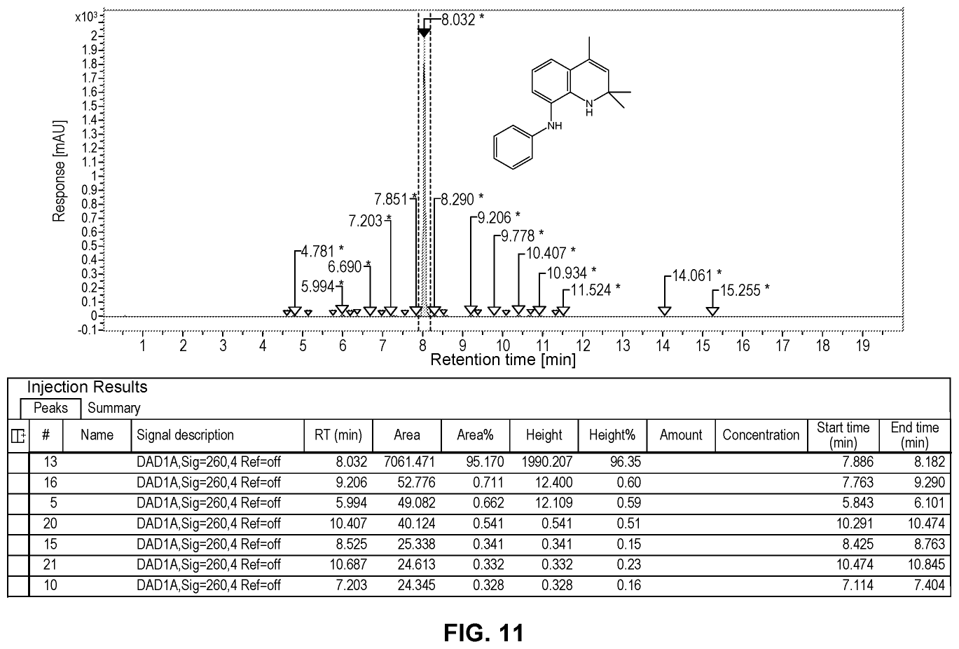

Fraction 1: Compound (2); isolated yield=0.37 g; light brown oil, which solidified upon standing at room temperature overnight. The structure was authenticated by NMR spectroscopy, as well as LC-MS m/z [M+1]=265. The purity was 95.2 area % as measured by LC-UV 260 nm. The carbon 13 APT NMR spectrum (DMSO-d₆) and HPLC chromatogram are depicted in FIG. 10 and FIG. 11, respectively.

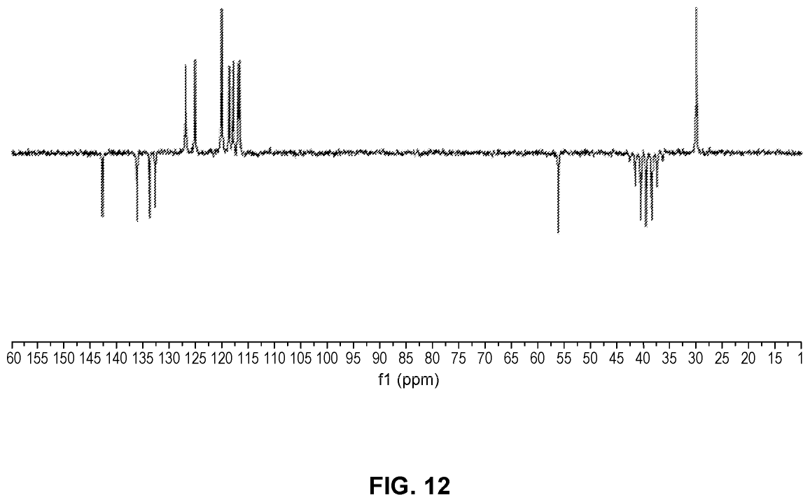

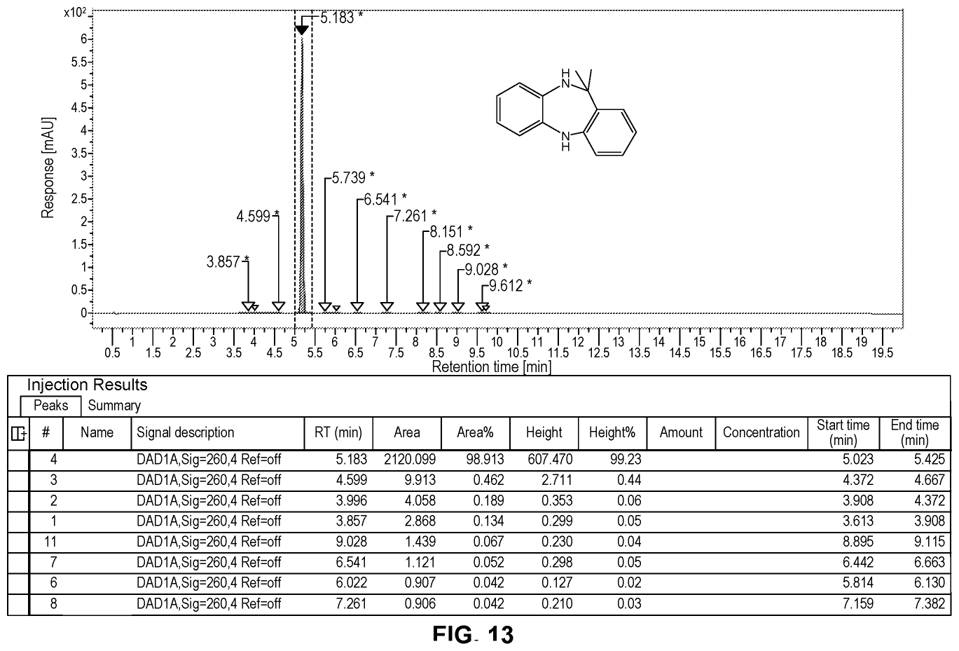

Fraction 2: isolated yield=9.2 g; light-orange solid. This impure material containing about 9% residual 2-ADPA (LC-UV 260 nm) was dissolved in AcOEt (14 mL) using a warm water bath. To the obtained solution under stirring and N2 protection was added hexanes (25 mL). The mixture was stirred until a solid separated. An AcOEt-hexanes mixture (7 mL and 11 mL, respectively) was added to loosen up the solid. The solid was collected by vacuum filtration over 0.2-micron disposable polyethylene frit and washed with an AcOEt-hexanes mixture (4 mL and 7 mL respectively). The damp beige solid was dried under vacuum at 50 deg C. for a few hours then at room temperature overnight. Yield=2.5 gas an almost white solid. The structure was authenticated by NMR spectroscopy, as well as LC-MS m/z [M+1]=225. The purity in Compound (21) was 98.9 area % as measured by LC-UV 260 nm. The carbon 13 APT NMR spectrum (DMSO-d₆) and HPLC chromatogram are depicted in FIG. 12 and FIG. 13, respectively.

### Example 9

### Synthesis of Compound (23) (1,2,2,4-tetramethyl-N-phenyl-1,2-dihydroquinolin-6-amine) and Compound (24) (N,1,2,2,4-pentamethyl-N-phenyl-1,2-dihydroquinolin-6-amine)

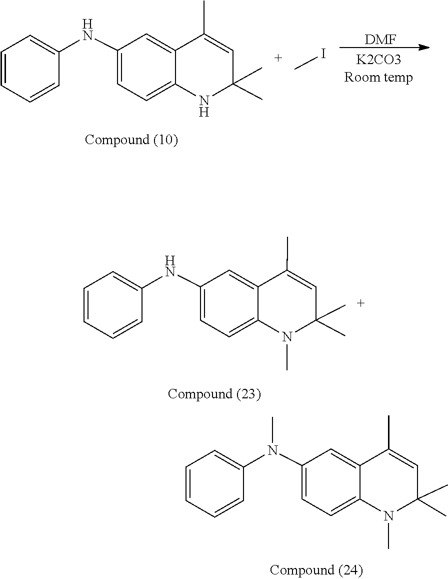

To a 250-mL round bottom flask fitted with a stir-bar was loaded Compound (10) (5.0 g; 18.9 mmol). The flask was put under N2 protection and dry DMF (27 mL) was added. Stirring was started. Once Compound (10) was fully dissolved, anhydrous potassium carbonate (3.317 g; 24.0 mmol) was added. The flask was cooled with a cold tap water bath. Iodomethane (1.5 mL; 24.0 mmol) was added over 1-2 minutes. No noticeable exotherm occurred. The cooling bath was removed. The reaction mixture was stirred without external cooling or heating for 21.5 hours. Deionized water (27 mL) was added and the mixture was stirred for 30 minutes. AcOEt (100 mL) and deionized water (20 mL) were added and the mixture was stirred until dissolution of the gummy material. The obtained 2-phase system was transferred to a separatory funnel and the organic layer was collected. The aqueous layer was extracted with AcOEt (70 mL). The combined organic layers were washed with aqueous 1 mol/L NaOH (3×50 mL) and aqueous NaCl, dried over MgSO₄, filtered, then stripped of volatiles under reduced pressure (rotary evaporator; water bath=50 deg C.). Further drying on the rotary evaporator (60 deg C.; <10 mbars; 1 hour) afforded the crude material (brown oil; 4.1 g). The crude material was dissolved in 5% AcOEt in hexanes (7 mL). The obtained solution was loaded into a 41 mm inner diameter column prepared with silica gel 60 and 5% AcOEt in hexanes. The height of wet silica gel in the column was 25-26 cm. The column was eluted with 5% AcOEt in hexanes. Each portion coming out was analyzed by silica gel TLC. Fraction 1 containing the material having a Rf of 0.4 (5% AcOEt in hexanes) was collected. The column was eluted with 20% AcOEt in hexanes. Fraction 2 containing the material having a Rf of 0.15 (5% AcOEt in hexanes) was collected. Each fraction was independent stripped of volatiles under reduced pressure (rotary evaporator; water bath=50 deg C.). Further drying on the rotary evaporator (60 deg C.; 9-10 mbars) until constant weight afforded the final products.

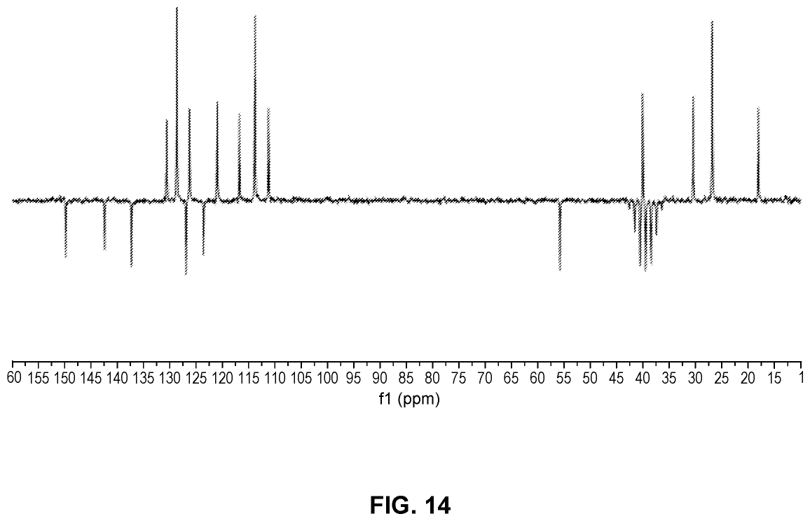

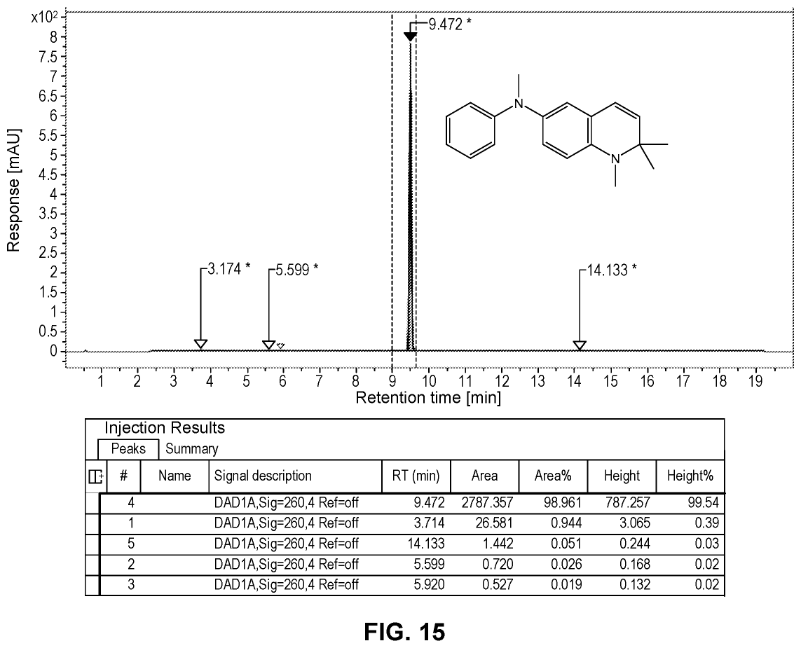

Fraction 1: Compound (24); isolated yield=0.8 g; light yellow oil. The structure was authenticated by NMR spectroscopy, as well as LC-MS m/z [M+1]=293. The purity was 99.0 area % as measured by LC-UV 260 nm. The carbon 13 APT NMR spectrum (DMSO-d₆) and HPLC chromatogram are depicted in FIG. 14 and FIG. 15, respectively.

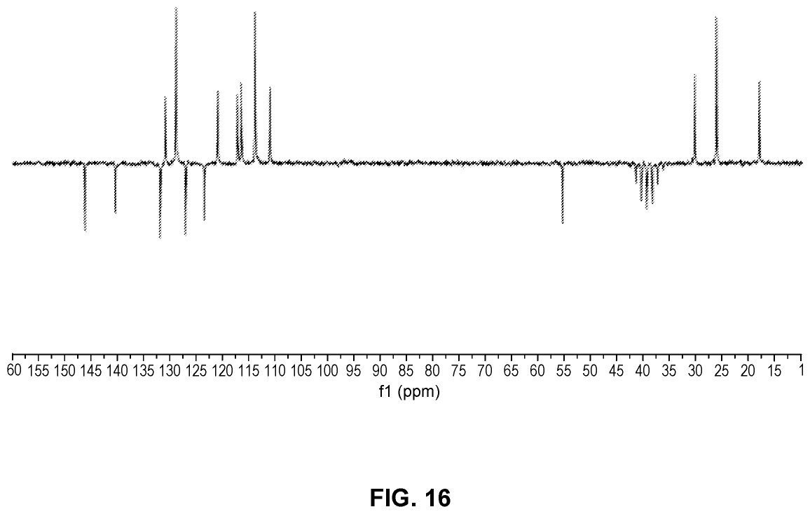

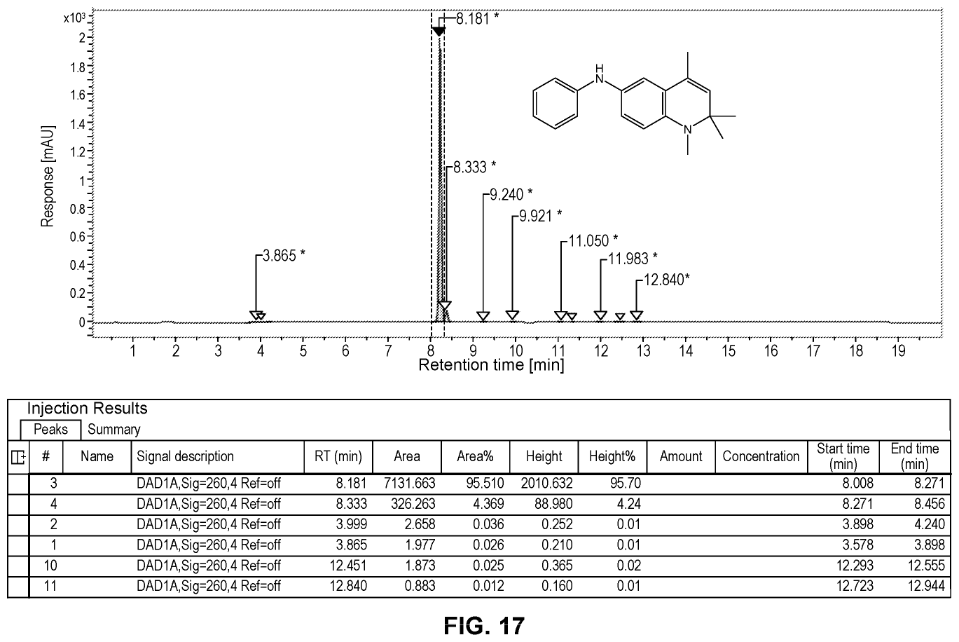

Fraction 2: Compound (23); isolated yield=3.2 g; light yellow oil. The structure was authenticated by NMR spectroscopy, as well as LC-MS m/z [M+1]=279. The purity was 95.5 area % as measured by LC-UV 260 nm. The carbon 13 APT NMR spectrum (DMSO-d₆) and HPLC chromatogram are depicted in FIG. 16 and FIG. 17, respectively.

### Example 10

### Synthesis of Compound (7) (2,2-dimethyl-1-phenyl-2,3-dihydro-1H-benzo[d]imidazole)

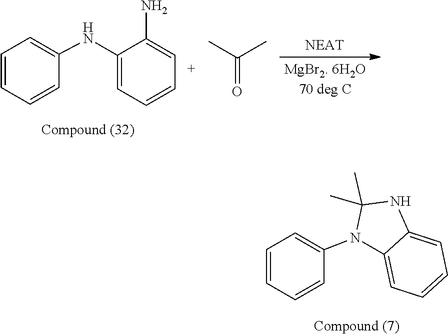

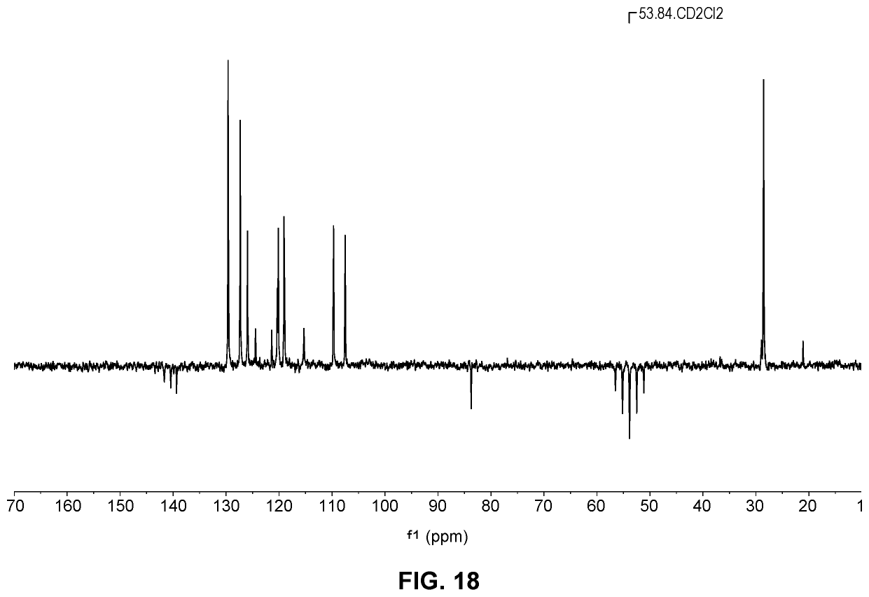

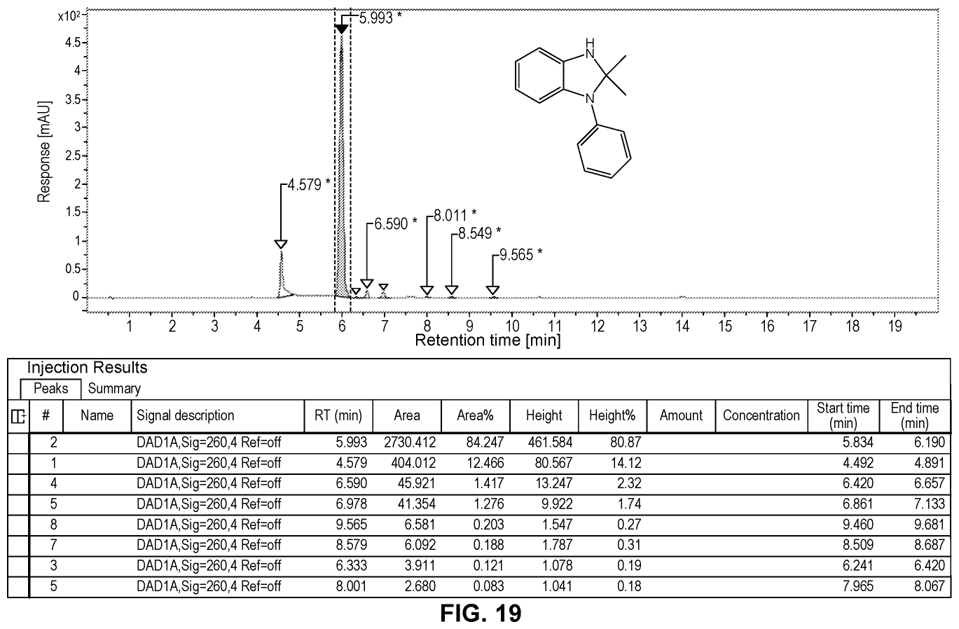

To a 2-DR vial fitted with a stir-bar was loaded Compound (32) (1.632 g; 8.86 mmol), magnesium bromide hexahydrate (270 mg; 0.924 mmol), and acetone (3 mL; 40.9 mmol). The vial was purged with N2 for a few seconds, capped, then heated to 70 deg C. (heating block) for 17 hours. Upon cooling to room temperature, part of the mixture post reaction was directly loaded into a 1-inch inner diameter column prepared with neutral alumina (grade I) and 10% AcOEt in hexanes. The height of wet alumina in the column was 11-12 cm. The column was eluted with 10% AcOEt in hexanes. Each fraction coming out was analyzed by neutral alumina TLC (20% AcOEt in hexanes). The fractions containing the dot at Rf=0.6 were collected, combined, and stripped of volatiles under reduced pressure (rotary evaporator; water bath=45 deg C.). Further drying on the rotary evaporator (45 deg C.) until constant weight afforded a light-brown oil (0.2 g), which solidified as a beige solid upon standing to room temperature overnight. The structure was authenticated by NMR spectroscopy, as well as LC-MS m/z [M+1]+=225. The purity in Compound (7) was 84.2 area % as measured by LC-UV 260 nm (the chromatogram also shows 12.5 area % of compound (32)). The carbon 13 APT NMR spectrum (CD₂Cl₂) and HPLC chromatogram are depicted in FIG. 18 and FIG. 19, respectively.

### Example 11

### Synthesis of Compound (25) (N,2,2,4-tetramethyl-N-phenyl-1,2-dihydroquinolin-6-amine)

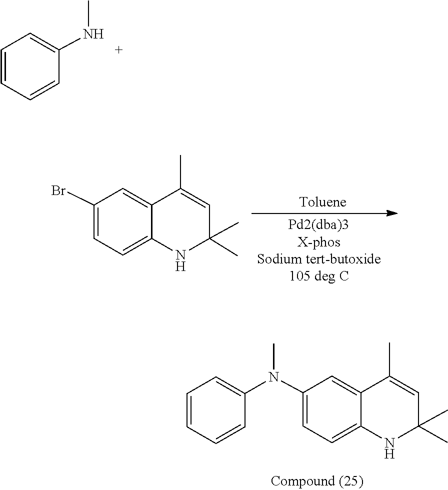

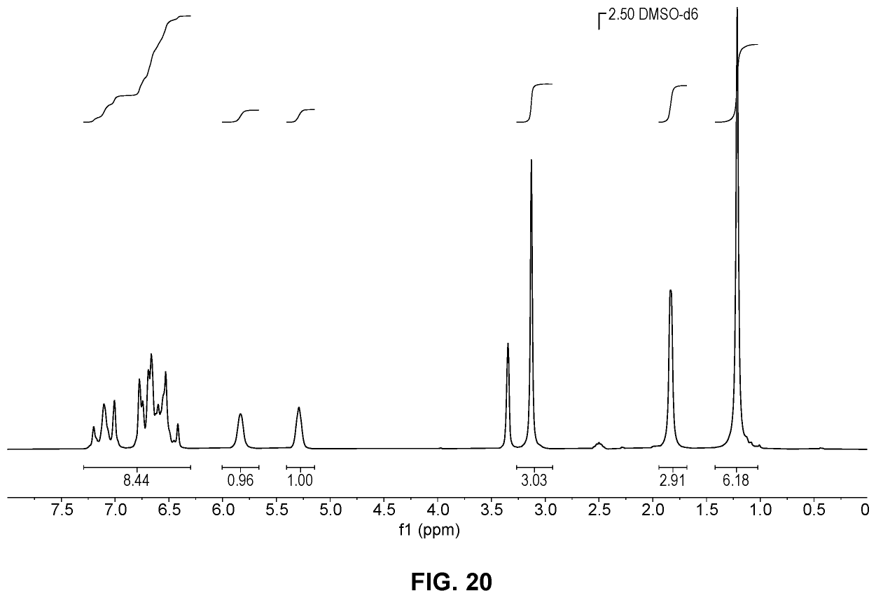

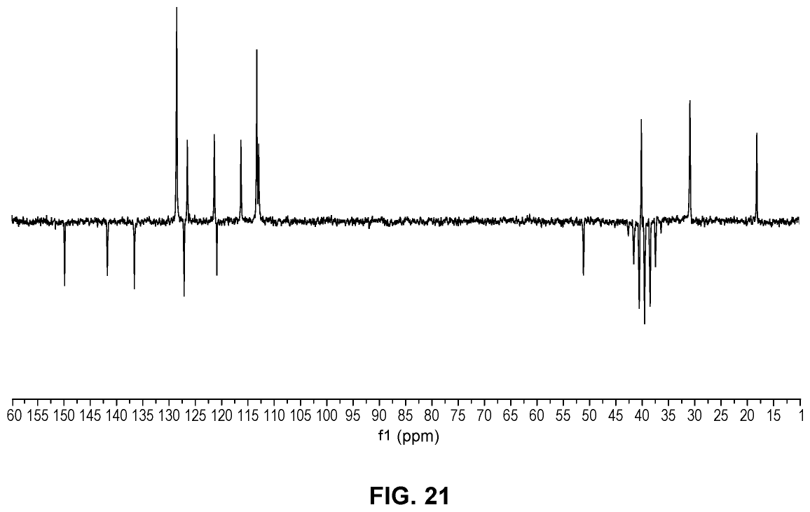

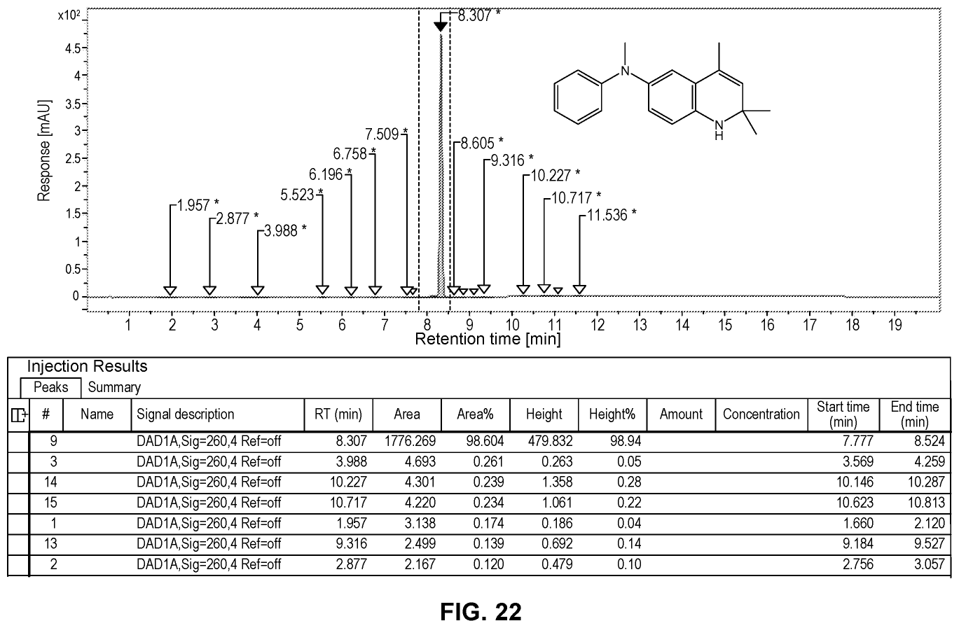

To a 250-mL round bottom flask fitted with a stir-bar and a reflux condenser was charged the substrate 6-bromo-2,2,4-trimethyl-1,2-dihydroquinoline (1.415 g; 5.61 mmol), anhydrous grade toluene (20.5 mL), and N-methylaniline (0.63 mL; 5.81 mmol). The mixture was stirred under N2 blanket. Once the substrate was fully dissolved (a few minutes), dicyclohexyl(2′,4′,6′-triisopropyl-[1,1′-biphenyl]-2-yl)phosphane (0.1159 g; 0.243 mmol), tris(dibenzylideneacetone)dipalladium(0) (0.1238 g; 0.135 mmol), and sodium tert-butoxide (0.6087 g; 6.33 mmol) were sequentially added to the flask. N2 was bubbled through the mixture for a minute or so, then the reaction mixture was heated (oil bath temperature set to 105 deg C.) under N2 blanket for 3.5 hours. Upon cooling to room temperature (overnight), the mixture post-reaction was filtered under suction over Celite S. The cake was rinsed with toluene. The combined filtrates were stripped of volatiles under reduced pressure (rotary evaporator; water bath=70 deg C.). The obtained brown oil was diluted with hexanes (4 mL) and the obtained solution was loaded into a 1-inch inner diameter column prepared with silica gel 60 and 5% AcOEt in hexanes. The height of wet silica gel in the column was approx. 18 cm. The column was eluted with 5% AcOEt in hexanes. Each fraction coming out was analyzed by silica gel TLC (5% AcOEt in hexanes). The fractions containing the dot at Rf=0.2 were combined and stripped of volatiles under reduced pressure (rotary evaporator; water bath=50 deg C.). Drying under vacuum at 50 deg C. for a few hours then at room temperature overnight afforded 1.212 g of a very viscous light-brown oil. The structure was authenticated by NMR spectroscopy, as well as LC-MS m/z [M+1]+=279. The purity in Compound (25) was 98.6 area % as measured by LC-UV 260 nm. The proton and carbon 13 APT NMR spectra (DMSO-d₆), as well as HPLC chromatogram are depicted in FIG. 20, FIG. 21, and FIG. 22, respectively.

### Example 12

### Synthesis of Compound (8) (1-phenyl-1H-benzo[d]imidazole)

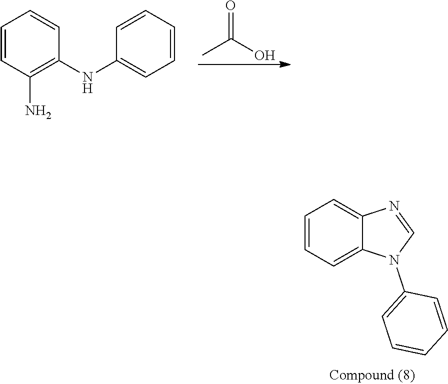

To a stirred solution of formic acid (425 mL) added 2-amino-diphenylamine (85.0 g, 0.46 mol) at room temperature. Reaction was heated to 100° C. and maintained for 16 h. After completion of the reaction (monitored by TLC), cooled to room temperature and reaction mass dissolved in DCM (500 mL), washed with water (2×200 mL) followed by Sat. NaHCO₃ solution. Separated organic layer and evaporated under reduced pressure at 45° C. to afford crude compound of 75.0 g Compound (8).

Purification: The obtained the crude compound was purified by column chromatography using silica gel (60-120 mesh) as the stationary phase, eluting with 0-15% ethyl acetate in hexane, collected the pure fractions and evaporated under reduced pressure to afford desired 55.0 g of Compound (8) as a brown semisolid.

¹H NMR (400 MHz, DMSO-d₆): δ 8.57 (s, 1H), 7.80 (dd, J=4.8 Hz, 1H), 7.79-760 (m, 1H), 7.51 (t, J=7.2 Hz, 1H), 7.49-7.29 (m, 1H). ¹³C NMR (100 MHz, DMSO-d₆): δ 147.39, 143.71, 136.46, 133.56, 130.50, 128.13, 124.08, 123.91, 122.92, 120.48, 111.08, 40.63, 40.42, 40.00, 39.80, 39.59, 39.38.

### Example 13

### Synthesis of Compound (34) (N-(2-(phenylamino)phenyl)formamide)

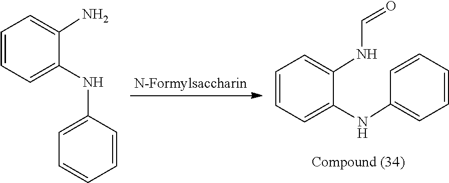 [SMILES: Nc1ccccc1Nc1ccccc1 ; O=CNc1ccccc1Nc1ccccc1]

To a stirred solution of 2-amino-diphenylamine (2.5 g, 13.58 mmol) in THE (25 mL, 10 vol), was added N-Formyl saccharin (2.8 g, 13.58 mmol) at RT, where it was stirred for 15-20 min. After completion of reaction (monitored by TLC), quenched with sat. NaHCO₃ solution (12 mL) and extracted with DCM (30 mL). Separated the organic layer, dried over sodium sulphate, filtered and evaporated under vacuum to afford 3.4 g of crude Compound (34). The obtained crude product was purified by silica-gel (100-200 mesh) column chromatography eluting with 15-20% ethyl acetate in hexane and pure fractions were collected and solvent was evaporated under reduced pressure to afford pure Compound (34) (observed as Rotamers by ¹H NMR) as an off-white solid. Yield 1.5 g.

¹H NMR (400 MHz, DMSO-d₆at 90° C.): 6 9.21 (s, 1H), 8.32 (s, 1H), 7.73 (br s, 1H), 7.22-7.15 (m, 3H), 7.10-7.06 (m, 2H), 7.01-6.97 (m, 1H), 6.84-6.82 (m, 2H), 6.80-6.76 (m, 1H). ¹³C NMR (100 MHz, DMSO-d₆): δ 163.87, 160.73, 145.31, 144.41, 135.74, 134.54, 130.88, 129.60, 129.53, 126.25, 125.45, 123.55, 123.22, 122.63, 122.20, 120.52, 119.96, 119.50, 116.79, 116.21.

### Example 14

### Synthesis of Compound (37) (2-methyl-1-phenyl-1H-benzo[d]imidazole)

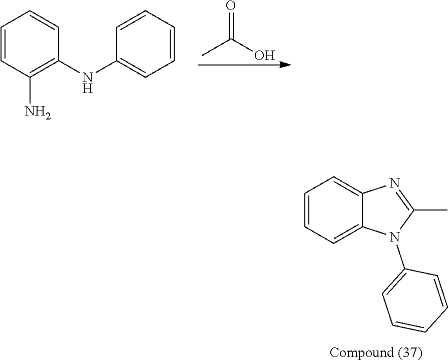

To a stirred solution of acetic acid (500 mL) added 2-amino-diphenylamine (100.0 g, 0.54 mol) at room temperature. Heated to 90° C. and maintained for 16 h. After completion of the reaction (monitored by TLC), cooled to room temperature and reaction mass dissolved in DCM (500 mL), washed with water (2×200 mL) followed by Sat. NaHCO₃ solution. Separated organic layer and evaporated under reduced pressure at 45° C. to afford crude compound of 110.0 g Compound (37).

Purification: The obtained the crude compound was purified by column chromatography using silica gel (60-120 mesh) as the stationary phase, eluting with 0-15% ethyl acetate in hexane, collected the pure fractions and evaporated under reduced pressure to afford desired 60.0 g of Compound (37) as a light pink color solid.

¹H NMR (400 MHz, DMSO-d₆): δ 7.66 (t, J=7.2 Hz, 3H), 7.64-7.53 (m, 3H), 7.24-7.15 (m, 2H), 7.10 (d, J=7.2 Hz, 1H), 2.43 (s, 3H). ¹³C NMR (100 MHz, DMSO-d₆): δ 151.74, 142.90, 136.07, 130.43, 129.12, 127.34, 122.75, 122.39, 118.97, 110.21, 40.67, 40.63, 40.46, 40.42, 40.25, 40.21, 40.00, 39.79, 39.58, 14.57.

### Example 15

### Synthesis of Compound (38) (N1-methyl-N1-phenylbenzene-1,4-diamine)

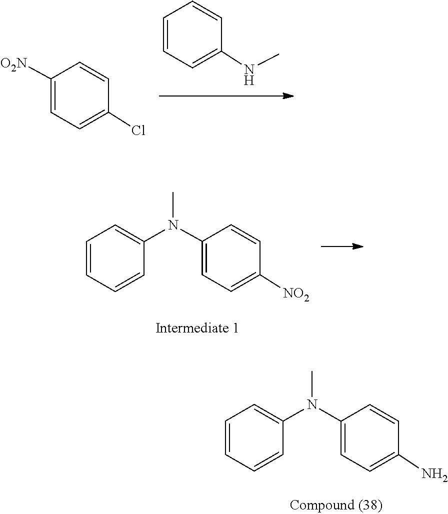

To a stirred and degassed solution of toluene (2000 mL) was added Pd₂dba₃ (23.2 g, 0.025 mol), X-Phos (6 g, 0.012 mol), at RT and degassed for 15 min., then reaction contents were heated to 60° C. Then added p-chloronitrobenzene (100 0.0 g, 0.63 mol) and N-methyl aniline (75 g, 0.69 mol) followed by added NaOtBu (91.5 g, 0.95 mol). The resulting reaction mixture was heated to 90° C. and stirred for 16 h. Completion of the reaction, monitored by TLC, cooled to RT and filtered through celite pad and washed with ethyl acetate (300 mL). The combined filtrate was washed with water, dried over Na₂SO₄ and evaporated under reduced pressure to afford crude Intermediate 1 (75.0 g).

Purification: The above obtained crude product was purified by column chromatography using silica gel (100-200 mesh) as the stationary phase, eluting with 5-10% ethyl acetate in hexane. Collected the pure fractions and evaporated solvent under vacuum to afford desired 60.0 g of Intermediate 1 as a brown solid.

¹H NMR (400 MHz, DMSO-d₆): δ 7.54 (s, 1H), 7.10 (td, J=8.4, 1.6 Hz, 2H), 6.82 (dd, J=8.8, 1.2 Hz, 2H), 6.63-6.56 (m, 3H), 6.49 (dd, J=8.0, 2.0 Hz, 1H), 4.38 (s, 2H), 3.72 (s, 3H).

### Step-2

To a stirred solution of Intermediate 1 (60 g, 0.26 mol) in methanol (300 mL) added Raney-N1 (15 g, 25% w/w) at RT. Then hydrogen pressure was applied (10 kg) and maintained for 16 h. After completion of the reaction (monitored by TLC), filtered the reaction mixture through celite pad, washed with methanol (150 mL) and solvent was evaporated to afford 55.0 g of Compound 38 as a crude product.

Purification: The above obtained crude product was purified by column chromatography using silica gel (100-200 mesh) as the stationary phase, eluting with 15% ethyl acetate in hexane. Collected the pure fractions and solvent was evaporated under vacuum to afford desired 44.0 g of Compound (38) as a brown solid.

¹H NMR (400 MHz, DMSO-d₆): δ 7.11-7.06 (m,2 H), 6.85-6.81 (m, 2H), 6.63-6.56 (m, 5H), 5.03 (s, 2H), 3.11 (s, 3H). ¹³C NMR (100 MHz, DMSO-d₆): δ 150.49, 146.71, 137.65, 129.12, 127.52, 117.02, 115.27, 114.06.

### Example 16

Synthesis of Compound (39) (N1-methyl-N4-phenylbenzene-1,4-diamine)

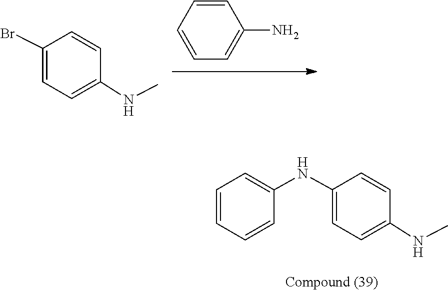

To a stirred and degassed solution of toluene (1000 mL) was added Pd₂dba₃ (18.6 g, 0.02 mol), X-Phos (4.8 g, 0.01 mol), at RT and degassed for 15 min., then reaction mixture was heated to 60° C. and added 4-bromo-N-methylaniline (95 0.0 g, 0.30 mol), aniline (53 mL, 0.6 mol) followed by added NaOtBu (73 g, 0.76 mol). The resulting reaction mixture was heated to 90° C. and stirred for 16 h. Completion of the reaction monitored by TLC, cooled to RT and filtered through celite pad and washed with ethyl acetate (250 mL). The combined filtrate was washed with water, dried over Na₂SO₄ and solvent was evaporated under reduced pressure to afford crude compound (39) 125.0 g.

Purification: The above obtained crude product was purified by column chromatography using silica gel (100-200 mesh) as the stationary phase, eluting with 5-10% ethyl acetate in hexane. Collected the pure fractions and evaporated under vacuum to afford 42.0 g of compound (39) as a light brown solid.

¹H NMR (400 MHz, DMSO-d₆): δ 7.48 (s, 1H), 7.09 (t, J=8.4 Hz, 2H), 6.90 (d, J=8.4 Hz, 2H), 6.77 (d, J=7.6 Hz, 2H), 6.59 (t, J=7.2 Hz, 1H), 6.59 (d, J=8.4 Hz, 2H), 5.38 (q, J=10.0 Hz, 1H), 2.65 (d, J=5.2 Hz, 3H). ¹³C NMR (100 MHz, DMSO-d₆): δ 147.17, 146.07, 131.91, 129.43, 123.09, 117.47, 114.08, 112.84, 30.72

### Example 17

### Synthesis of Compound (40) (3-methoxy-N1-phenylbenzene-1,4-diamine)

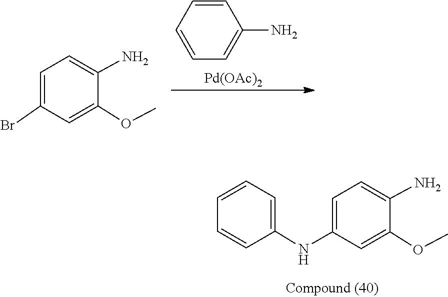

Purged nitrogen into 1,4-Dioxane (250 mL) for 30 min, followed by the addition of Pd(OAc)₂ (2.80 g, 0.012 mol) and X-Phos (17.7 g, 0.036 mol). After purging the resulting reaction mixture with nitrogen for an additional 15-20 min, then 4-bromo-2-methoxyaniline (25.0 g, 0.123 mol) and aniline (34.0 mL, 0.371 mol) were added followed by the addition of cesium carbonate (121.0 g, 0.371 mol). The resulting reaction contents were heated at 85-90° C. and stirred for 2-3 h. After completion of reaction (monitored by TLC), the reaction mixture was cooled to RT and filtered through a celite pad. The filter cake was thoroughly washed with 1,4-Dioxane and the filtrate was evaporated under reduced pressure to get crude residue (46 g). The obtained residue was further purified by silica-gel (100-200 mesh) column chromatography eluting with 10-15% ethyl acetate in hexane to afford 8.7 g of compound (40) as a light purple solid.

¹H NMR (400 MHz, DMSO-d₆): δ 7.54 (s, 1H), 7.10 (td, J=8.4, 1.6 Hz, 2H), 6.82 (dd, J=8.8, 1.2 Hz, 2H), 6.63-6.56 (m, 3H), 6.49 (dd, J=8.0, 2.0 Hz, 1H), 4.38 (s, 2H), 3.72 (s, 3H). ¹³C NMR (100 MHz, DMSO-d₆): δ 147.31, 146.83, 132.95, 132.91, 129.44, 117.69, 114.71, 114.41, 113.80, 105.55, 55.66.

### Example 18

### Synthesis of Compound (49) (2,4-dimethyl-N-phenylquinolin-6-amine)

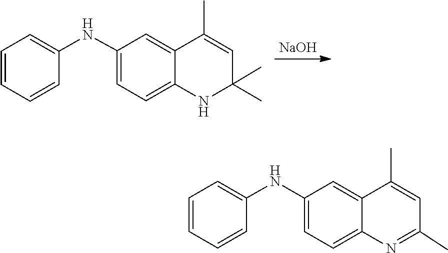

A mixture of compound (10) (50.0 g, 0.189 mol) and sodium hydroxide (200.0 g, 400% w/w) was heated at 140-150° C. and stirred for 16 h at the same temperature. After 16 h, cooled to RT, quenched with water and extracted with DCM (500 mL). Separated the organic layer, dried over sodium sulphate, filtered and evaporated under vacuum to afford 60.0 g of crude compound (49).

Purification: The above obtained crude product was purified by silica-gel (100-200 mesh) column chromatography eluting with 45-50% ethyl acetate in hexane and pure fractions were collected and evaporated solvent under reduced pressure to afford 6.9 g of compound (49) as light brown solid.

¹H NMR (400 MHz, CDCl₃): δ 7.92 (d, J=9.2 Hz, 1H), 7.50 (d, J=2.8 Hz, 1H), 7.42 (dd, J=9.2, 2.8 Hz, 1H), 7.34-7.29 (m, 2H), 7.17 (d, J=7.2 Hz, 2H), 7.07 (s, 1H), 6.99 (t, J=7.2 Hz, 1H), 5.96 (s, 1H), 2.65 (s, 3H), 2.55 (s, 3H). ¹³C NMR (100 MHz, CDCl₃): δ 151.06, 138.85, 138.13, 137.85, 135.86, 125.38, 124.76, 122.88, 118.31, 117.96, 116.73, 113.34, 113.19, 102.71, 20.09, 13.96.

### Example 19

### Oxidative Induction Time (OIT) Performance

The oxidative induction time (OIT) of representative Compounds of the Disclosure were evaluated. OIT is measured according to a procedure carried out in a differential scanning calorimeter (DSC; TA Instruments, Q200). In this procedure, a sample is held in a cell and heated under a nitrogen atmosphere to a preselected temperature. Oxygen (O₂) is then introduced to the cell and the length of time before the onset of degradation, as observed by the initiation of an endothermic process in the DSC trace, is measured.

0.5 wt % of the Compound of the Disclosure or no antidegradant (blank) was mixed with polyisoprene and heated isothermally at 150° C. and/or 160° C. in 02. The OIT in minutes is shown in Table 8. As indicated by these data, Compounds of the Disclosure demonstrate antioxidant activity.

TABLE 8

| Material | OIT at 150° C. (minutes) | OIT at 160° C. (minutes) |
|---|---|---|
| Blank | 1.8 | 1.4 |
| Compound (2) | 35.2 | 10.9 |
| Compound (4) | 27.2 | 12.1 |
| Compound (7) | 24.2 | 11.8 |
| Compound (8) | 2.2 | 1.1 |
| Compound (10) | 60.6 | 26.6 |
| Compound (21) | 25.5 | 12.3 |
| Compound (23) | 84.9 | 36.3 |
| Compound (24) | 31.0 | 16.0 |
| Compound (25) | 23.3 | 11.3 |
| Compound (32) | 4.9 | 2.1 |
| Compound (33) | 2.1 | 1.2 |
| Compound (34) | 7.0 | 3.3 |
| Compound (35) | 2.1 | 1.2 |
| Compound (36) | 1.6 | 1.1 |
| Compound (37) | 1.8 | 1.2 |
| Compound (38) | 5.2 | 2.2 |
| Compound (39) | 17.0 | 7.2 |
| Compound (40) | 32.7 | 11.9 |
| Compound (49) | 23.1 | 12.9 |

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof All patents, patent applications, and publications cited herein are fully incorporated by reference herein in their entirety.

What is claimed is:

1. A compound selected from the group consisting of Compound (2), Compound (21), and Compound (25): 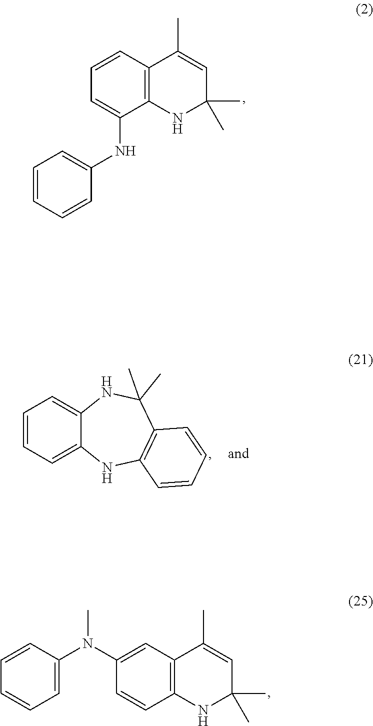 [SMILES: CC1=CC(C)(C)Nc2c(Nc3ccccc3)cccc21 ; CC1(C)Nc2ccccc2Nc2ccccc21 ; CC1=CC(C)(C)Nc2ccc(N(C)c3ccccc3)cc21]
or a salt or solvate thereof

2. The compound of claim 1 that is Compound (2).

3. The compound of claim 1 that is Compound (21).

4. The compound of claim 1 that is Compound (25).

5. A composition comprising:
(i) Compound (10): 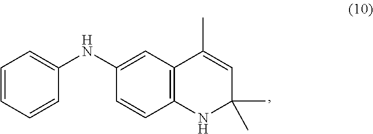 [SMILES: CC1=CC(C)(C)Nc2ccc(Nc3ccccc3)cc21]
or a salt or solvate thereof; and
(ii) one or more molecules, or a salt or solvate thereof,
wherein the one or more molecules are selected from the group consisting of: 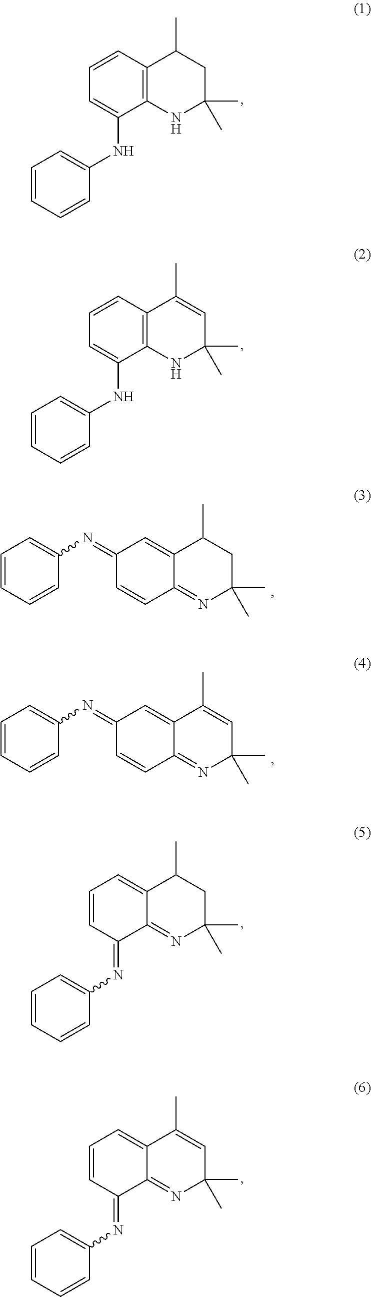 [SMILES: CC1CC(C)(C)Nc2c(Nc3ccccc3)cccc21 ; CC1=CC(C)(C)Nc2c(Nc3ccccc3)cccc21 ; CC1CC(C)(C)N=C2C=CC(=Nc3ccccc3)C=C21 ; CC1=CC(C)(C)N=C2C=CC(=Nc3ccccc3)C=C12 ; CC1CC(C)(C)N=C2C1=CC=CC2=Nc1ccccc1 ; CC1=CC(C)(C)N=C2C1=CC=CC2=Nc1ccccc1] 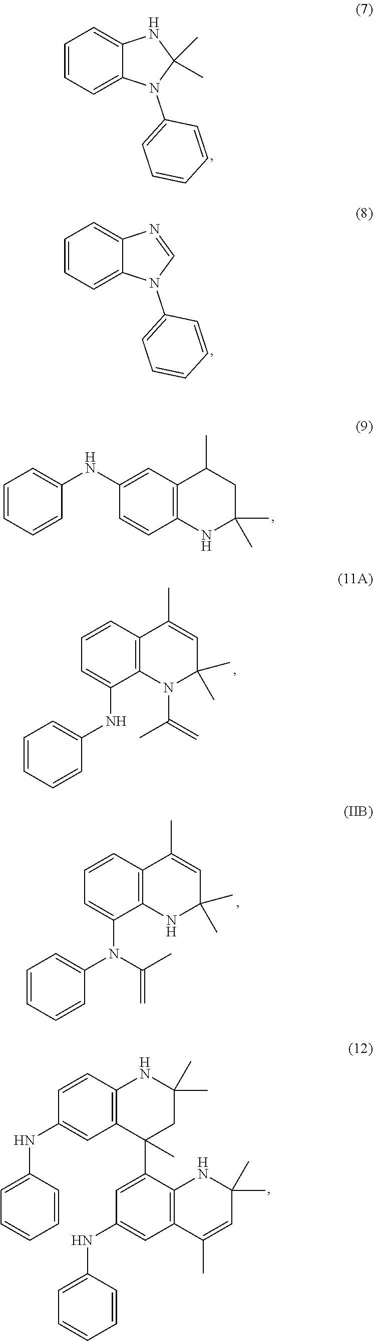 [SMILES: CC1(C)Nc2ccccc2N1c1ccccc1 ; c1ccc(-n2cnc3ccccc32)cc1 ; CC1CC(C)(C)Nc2ccc(Nc3ccccc3)cc21 ; C=C(C)N1c2c(Nc3ccccc3)cccc2C(C)=CC1(C)C ; C=C(C)N(c1ccccc1)c1cccc2c1NC(C)(C)C=C2C ; CC1=CC(C)(C)Nc2c1cc(Nc1ccccc1)cc2C1(C)CC(C)(C)Nc2ccc(Nc3ccccc3)cc21] 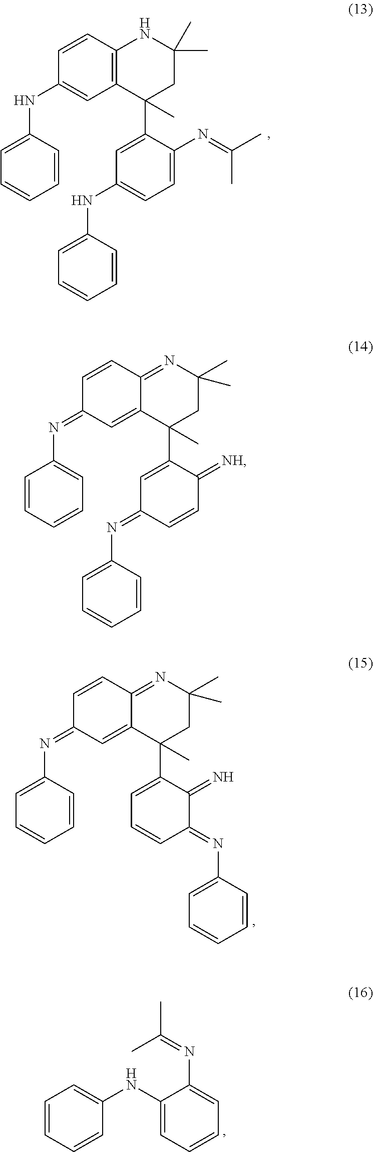 [SMILES: CC(C)=Nc1ccc(Nc2ccccc2)cc1C1(C)CC(C)(C)Nc2ccc(Nc3ccccc3)cc21 ; CC1(C)CC(C)(C2=C/C(=N/c3ccccc3)C=CC2=N)C2=C/C(=N\c3ccccc3)C=CC2=N1 ; CC1(C)CC(C)(C2=CC=C/C(=N\c3ccccc3)C2=N)C2=C/C(=N\c3ccccc3)C=CC2=N1 ; CC(C)=Nc1ccccc1Nc1ccccc1] 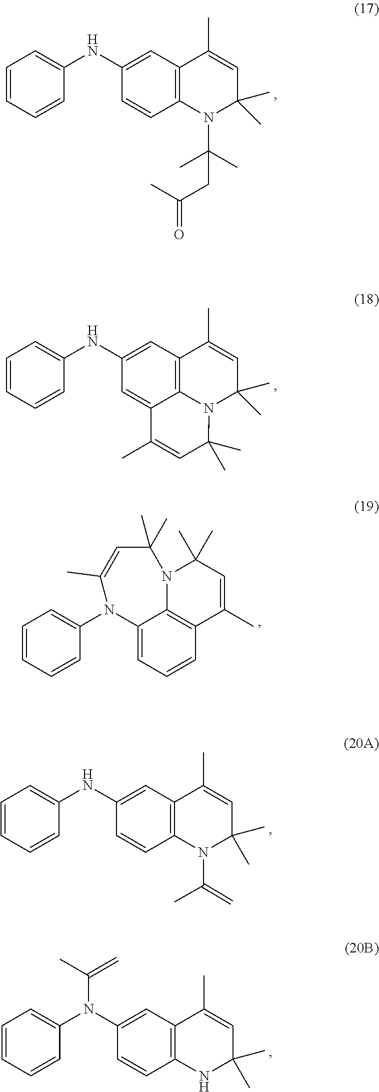 [SMILES: CC(=O)CC(C)(C)N1c2ccc(Nc3ccccc3)cc2C(C)=CC1(C)C ; CC1=CC(C)(C)N2c3c1cc(Nc1ccccc1)cc3C(C)=CC2(C)C ; CC1=CC(C)(C)N2c3c1cccc3N(c1ccccc1)C(C)=CC2(C)C ; C=C(C)N1c2ccc(Nc3ccccc3)cc2C(C)=CC1(C)C ; C=C(C)N(c1ccccc1)c1ccc2c(c1)C(C)=CC(C)(C)N2] 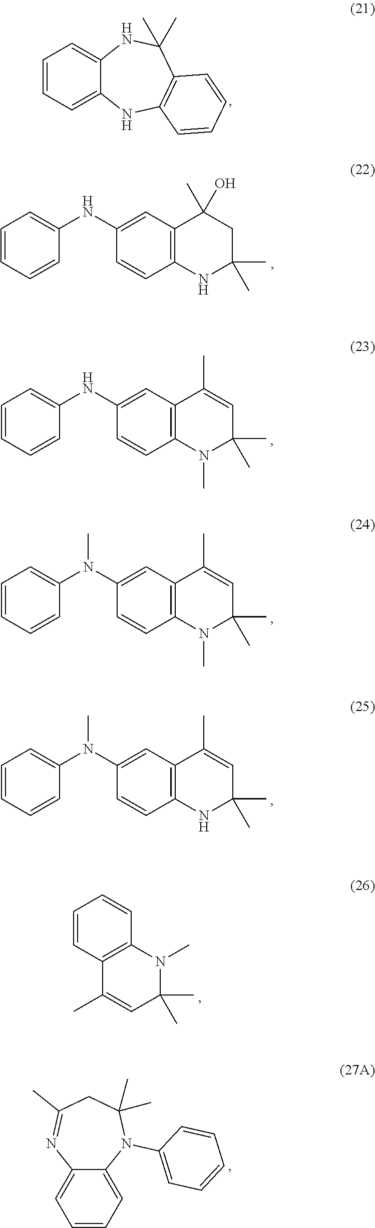 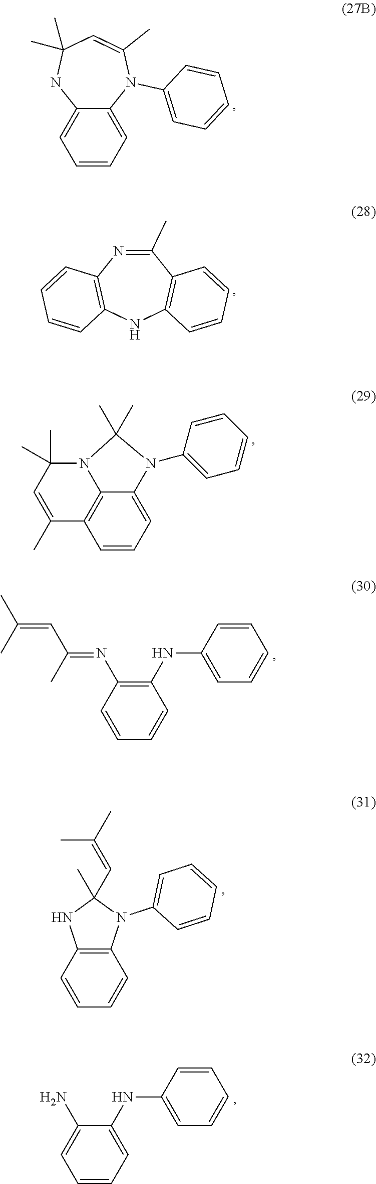 [SMILES: CC1=CC(C)(C)Nc2ccccc2N1c1ccccc1 ; CC1=Nc2ccccc2Nc2ccccc21 ; CC1=CC(C)(C)N2c3c1cccc3N(c1ccccc1)C2(C)C ; CC(C)=C/C(C)=N/c1ccccc1Nc1ccccc1 ; CC(C)=CC1(C)Nc2ccccc2N1c1ccccc1 ; Nc1ccccc1Nc1ccccc1] 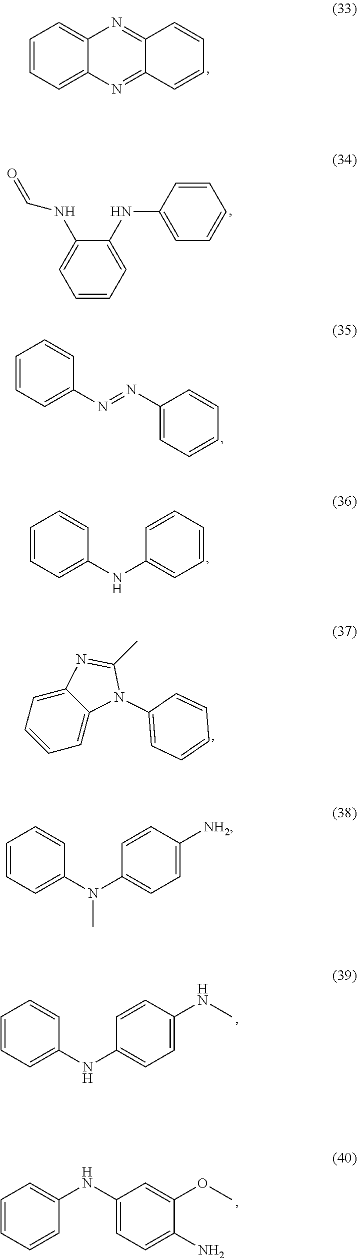 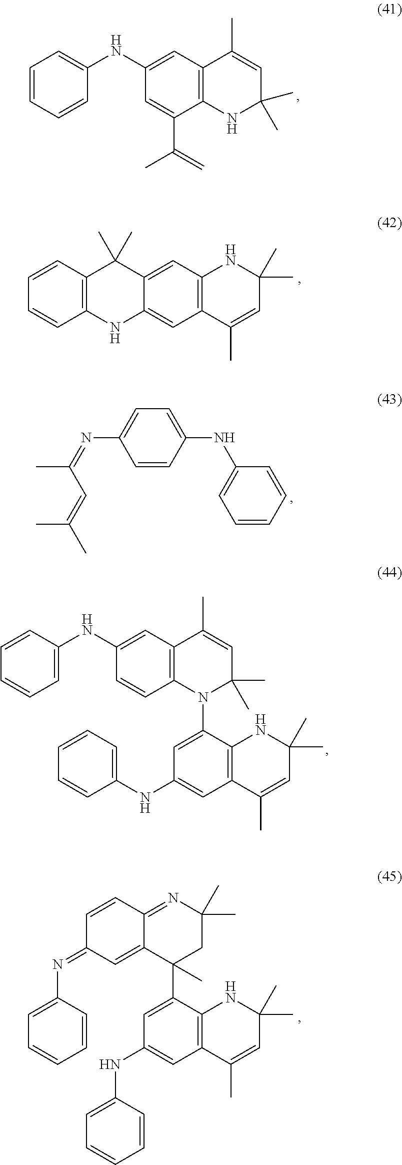 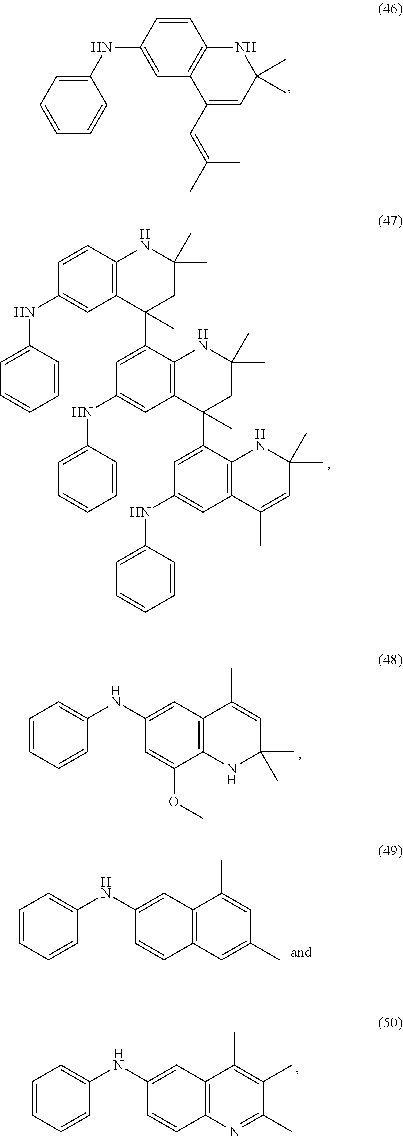
or a combination thereof.

6. The composition of claim 5, wherein the composition comprises Compound (1) as a molecule.

7. The composition of claims 5 or 6, wherein the composition comprises Compound (2) as a molecule.

8. The composition of any one of claims 5-7, wherein the composition comprises Compound (3) as a molecule.

9. The composition of any one of claims 5-8, wherein the composition comprises Compound (4) as a molecule.

10. The composition of any one of claims 5-9, wherein the composition comprises Compound (5) as a molecule.

11. The composition of any one of claims 5-10, wherein the composition comprises Compound (6) as a molecule.

12. The composition of any one of claims 5-11, wherein the composition comprises Compound (7) as a molecule.

13. The composition of any one of claims 5-12, wherein the composition comprises Compound (8) as a molecule.

14. The composition of any one of claims 5-13, wherein the composition comprises Compound (9) as a molecule.

15. The composition of any one of claims 5-14, wherein the composition comprises Compound (11A) as a molecule.

16. The composition of any one of claims 5-15, wherein the composition comprises Compound (11B) as a molecule.

17. The composition of any one of claims 5-16, wherein the composition comprises Compound (12) as a molecule.

18. The composition of any one of claims 5-17, wherein the composition comprises Compound (13) as a molecule.

19. The composition of any one of claims 5-18, wherein the composition comprises Compound (14) as a molecule.

20. The composition of any one of claims 5-19, wherein the composition comprises Compound (15) as a molecule.

21. The composition of any one of claims 5-20, wherein the composition comprises Compound (16) as a molecule.

22. The composition of any one of claims 5-21, wherein the composition comprises Compound (17) as a molecule.

23. The composition of any one of claims 5-22, wherein the composition comprises Compound (18) as a molecule.

24. The composition of any one of claims 5-23, wherein the composition comprises Compound (19) as a molecule.

25. The composition of any one of claims 5-24, wherein the composition comprises Compound (20A) as a molecule.

26. The composition of any one of claims 5-25, wherein the composition comprises Compound (20B) as a molecule.

27. The composition of any one of claims 5-26, wherein the composition comprises Compound (21) as a molecule.

28. The composition of any one of claims 5-27, wherein the composition comprises Compound (22) as a molecule.

29. The composition of any one of claims 5-28, wherein the composition comprises Compound (23) as a molecule.

30. The composition of any one of claims 5-29, wherein the composition comprises Compound (24) as a molecule.

31. The composition of any one of claims 5-30, wherein the composition comprises Compound (25) as a molecule.

32. The composition of any one of claims 5-31, wherein the composition comprises Compound (26) as a molecule.

33. The composition of any one of claims 5-32, wherein the composition comprises Compound (27A) as a molecule.

34. The composition of any one of claims 5-33, wherein the composition comprises Compound (27B) as a molecule.

35. The composition of any one of claims 5-34, wherein the composition comprises Compound (28) as a molecule.

36. The composition of any one of claims 5-35, wherein the composition comprises Compound (29) as a molecule.

37. The composition of any one of claims 5-36, wherein the composition comprises Compound (30) as a molecule.

38. The composition of any one of claims 5-37, wherein the composition comprises Compound (31) as a molecule.

39. The composition of any one of claims 5-38, wherein the composition comprises Compound (32) as a molecule.

40. The composition of any one of claims 5-39, wherein the composition comprises Compound (33) as a molecule.

41. The composition of any one of claims 5-40, wherein the composition comprises Compound (34) as a molecule.

42. The composition of any one of claims 5-41, wherein the composition comprises Compound (35) as a molecule.

43. The composition of any one of claims 5-42, wherein the composition comprises Compound (36) as a molecule.

44. The composition of any one of claims 5-43, wherein the composition comprises Compound (37) as a molecule.

45. The composition of any one of claims 5-44, wherein the composition comprises Compound (38) as a molecule.

46. The composition of any one of claims 5-45, wherein the composition comprises Compound (39) as a molecule.

47. The composition of any one of claims 5-46, wherein the composition comprises Compound (40) as a molecule.

48. The composition of any one of claims 5-47, wherein the composition comprises Compound (41) as a molecule.

49. The composition of any one of claims 5-48, wherein the composition comprises Compound (42) as a molecule.

50. The composition of any one of claims 5-49, wherein the composition comprises Compound (43) as a molecule.

51. The composition of any one of claims 5-50, wherein the composition comprises Compound (44) as a molecule.

52. The composition of any one of claims 5-51, wherein the composition comprises Compound (45) as a molecule.

53. The composition of any one of claims 5-52, wherein the composition comprises Compound (46) as a molecule.

54. The composition of any one of claims 5-53, wherein the composition comprises Compound (47) as a molecule.

55. The composition of any one of claims 5-54, wherein the composition comprises Compound (48) as a molecule.

56. The composition of any one of claims 5-55, wherein the composition comprises Compound (49) as a molecule.

57. The composition of any one of claims 5-56, wherein the composition comprises Compound (50) as a molecule

58. The composition of claim 5, wherein the composition comprises Compound (2) and Compound (21) as molecules.

59. The composition of claim 5, wherein the composition comprises Compound (2) and Compound (25) as molecules.

60. The composition of claim 5, wherein the composition comprises Compound (2) and Compound (37) as molecules.

61. The composition of claim 5, wherein the composition comprises Compound (21) and Compound (25) as molecules.

62. The composition of claim 5, wherein the composition comprises Compound (21) and Compound (37) as molecules.

63. The composition of claim 5, wherein the composition comprises Compound (25) and Compound (37) as molecules.

64. The composition of claim 5, wherein the composition comprises Compound (2), Compound (21), and Compound (25) as molecules.

65. The composition of claim 5, wherein the composition comprises Compound (2), Compound (21), and Compound (37) as molecules.

66. The composition of claim 5, wherein the composition comprises Compound (2), Compound (25), and Compound (37) as molecules.

67. The composition of claim 5, wherein the composition comprises Compound (21), Compound (25), and Compound (37) as molecules.

68. The composition of claim 5, wherein the composition comprises Compound (2), Compound (21), Compound (25), and Compound (37) as molecules.

69. The composition of any one of claims 5-68 comprising about 50% to about 99% of Compound (10) as measured by HPLC and/or GC analysis.

70. The composition of claim 69 comprising about 89% to about 99% of Compound (10) as measured by HPLC and/or GC analysis.

71. The composition of any one of claims 5-70 comprising about 0.000001% to about 10% of any one or more molecules as measured by HPLC and/or GC analysis.

72. The composition of claim 71 comprising about 0.0001% to about 5% of any one or more molecules as measured by HPLC and/or GC analysis.

73. The composition of claim 72 comprising about 0.001% to about 3% of any one or more molecules as measured by HPLC and/or GC analysis.

74. The composition of any one of claims 5-70, wherein the weight ratio of any one or more molecules to Compound (10) is 0.000001 to 0.1.

75. A composition comprising the compound of any one of claims 2-4 and
(i) one or more elastomers; or
(ii) one or more fillers; or
(iii) one or more rubber chemicals; or
(iv) one or more plasticizers; or
(v) one or more second antidegradants; or
(vi) a combination of one or more elastomers, one or more fillers, one or more rubber chemicals, one or more plasticizers, and/or one or more second antidegradants.

76. A composition comprising the composition of any one of claims 5-74 and
(i) one or more elastomers; or
(ii) one or more fillers; or
(iii) one or more rubber chemicals; or
(iv) one or more plasticizers; or
(v) one or more second antidegradants; or
(vi) a combination of one or more elastomers, one or more fillers, one or more rubber chemicals, one or more plasticizers, and/or one or more second antidegradants.

77. A vulcanized elastomeric article comprising the composition of claim 75 or 76.

78. A vehicle comprising the vulcanized elastomeric article of claim 77.

79. A kit comprising the composition of claim 77 or 78 and instructions for using the composition to prepare a vulcanized elastomeric article

80. A process for preparing the composition of any one of claims 5-74, the process comprising reacting a composition comprising 4-aminodiphenylamine with acetone, 2,2-dimethoxypropane, diacetone alcohol, or mesityl oxide in the presence of a catalyst.

81. The process of claim 80, wherein the catalyst is a homogeneous catalyst.

82. The process of claim 81, wherein the homogeneous catalyst is a Lewis acid, a Bronsted acid, a base, a halogen, a halogen donor, or a combination thereof.

83. The process of claim 81, wherein the homogeneous catalyst is selected from the group consisting of iodine, pyridinium perbromide, bromine, bismuth(III) trifluoromethane sulfonate, zinc(II) trifluoromethane sulfonate, hydrobromic acid, magnesium(II) bromide, aluminum(III) chloride, p-toluene sulfonic acid monohydrate, N-bromosuccinimide, copper(II) bromide, copper(II) chloride, methane sulfonic acid, benzene sulfonic acid, boron trifluoride diethyl etherate, boron trifluoride dihydrate, hydrochloric acid, copper(II) trifluoromethane sulfonate, iron(III) chloride, magnesium(II) chloride, copper(I) iodide, and zinc(II) chloride, or the salts or solvates thereof.

84. The process of claim 83, wherein the homogeneous catalyst is selected from the group consisting of pyridinium perbromide, magnesium(II) bromide, magnesium(II) bromide hexahydrate, and aluminum(III) chloride.

85. The process of claim 80, wherein the catalyst is a heterogeneous catalyst.

86. The process of claim 85, wherein the heterogeneous catalyst is an acid treated resin, a solid-supported acid, a composite catalyst, a solid acid, or a base catalyst.

87. The process of claim 86, wherein the acid treated resin is a polyfluorinated sulfonic acid resin, a strongly acidic sulfonic acid exchange resin (Amberlyst 15), a macroporous strong acid ion exchange resin, a strong acid ion exchange resin, a solid acidic ion exchange resin (NRW150), a sifted strong acidic ion exchange resin, or a strong acidic ion exchange resin (type A2 acidic resin).

88. The process of claim 86, wherein the solid-supported acid is micro-meso-macroporous zeolite catalyst H-Y-MMM, strong-acidity molecular sieve H-MCM-22, an organic protonic acid supported by graphene oxide, an organic protonic acid supported by a zeolite, a small pore size zeolite, a heterogeneous acidic silica catalyst, Zn²⁺, Sn²⁺, and Cu²⁺ exchanged tungstophosphoric acid (TPA) supported on γ-Al₂O₃, tonsil activated earth (acid treated bentonite clay), alumina, titania, zirconia, niobia, silica-alumina, sulfated zirconia, tungstated zirconia, para-toluenesulfonic acid (p-TSA)/ZnCl₂, or WO₃/activated carbon/SO₃H.

89. The process of claim 86, wherein the composite catalyst is zirconium doped titania composite oxide catalyst, H₂SO₄—MoO₃—TiO₂ solid superacid, or p-toluenesulfonic acid/iodine.

90. The process of claim 86, wherein the solid acid is a heteropolyacid or a metal-organic framework.

91. The process of claim 86, wherein the base catalyst is ZrO₂/MgO.
